(19) United States
(12) Patent Application Publication
(10) Pub. No.: US 2023/0374484 A1
(43) Pub. Date: Nov. 23, 2023

(54) Clotting Factor Variants and Their Use

(71) Applicants: Expression Therapeutics, LLC, Tucker, GA (US); Emory University, Atlanta, GA (US); Children's Healthcare of Atlanta, Atlanta, GA (US)

(72) Inventors: Gabriela D.C. Denning, Tucker, GA (US); Christopher B. Doering, Atlanta, GA (US); Harrison Brown, Tucker, GA (US); Kristopher Knight, Atlanta, GA (US); Christopher Coyle, Atlanta, GA (US); Harold Trent Spencer, Atlanta, GA (US)

(21) Appl. No.: 18/030,501

(22) PCT Filed: Dec. 29, 2021
(86) PCT No.: PCT/US2021/065588

§ 371 Date: Apr. 5, 2023

Related U.S. Application Data
(60) Provisional application No. 63/107,678, filed on Oct. 30, 2020.

(51) Int. Cl. C12N 9/64; A61P 7/04

(52) U.S. Cl. CPC C12N 9/644; C12Y 304/21022; A61P 7/04

(57) ABSTRACT

Disclosed herein are novel variants of clotting factor IX and their use, for example, in methods of treating a subject with a clotting disorder, such as hemophilia A or hemophilia B.

### RELATED APPLICATIONS

This application claims priority to U.S. Provisional Application No. 63/107,678, filed Oct. 30, 2020. The provisional patent application is incorporated by reference in its entirety.

### FIELD OF THE DISCLOSURE

This relates to novel clotting factor proteins, such as clotting factor IX, as well as recombinant nucleic acid molecules and vectors encoding the clotting factor proteins, and related methods of use to treat a clotting disorder, such as hemophilia, in a subject.

### BACKGROUND

Hemophilia B is associated with clotting factor IX (fIX). Treatment of clotting disorders such as hemophilia B typically entails lifelong, multi-weekly intravenous infusion of either human plasma-derived or recombinant clotting factors to replace the missing clotting factor activity in the patient. Due to the high cost, less than 30% of the global hemophilia population receives this form of treatment. Furthermore, about 25% of patients treated with clotting factor replacement products develop neutralizing antibodies that render future treatment ineffective. Thus, there is a need to identify improved therapies.

### SUMMARY

Disclosed herein are liver directed gene therapies for treatment of persons with hemophilia B with greater efficacy (higher or superior expression) than currently available gene therapies. Additionally, disclosed herein are variants of the fIX clotting factors with increased clotting factor activity relative to the corresponding native human clotting factor proteins. The variants of the fIX clotting factors have improved therapeutic properties, including improved procoagulant therapeutic properties, compared to an unmodified fIX polypeptide, including a human fIX polypeptide. The improved properties of the disclosed fIX variants include but are not limited to increased coagulation activity, increased catalytic activity, increased resistance to heparin, and/or improved pharmacokinetic properties. The improved properties may include decreased clearance rates, enhanced recovery, and etc.

Disclosed herein are novel fIX sequences consisting of a combination of amino acid substitutions in the EGF2 (V132A-V86A) and protease domain (N313S-N267S; E323K-E277K; V326T-V280T; D338N-D292N; K339R-K293R; H361N-H315N; K362R-K316R; L367S-L321S; L366S-L320S; V368I-V322I; V367I-V321I; F399Y-F353Y; R404K-R358K; D232N-D186N; V243L-V197L; V248I-V242I; V257I-V211I; I262V-I216V; V269I-V223I; T271P-225P; E286K-E240K; H289P-H243P; I299V-I253V; A308T-A262T; R384E-R338E and/or R384L-R338L) that confer enhanced fIX activity compared to human fIX by one-stage APTT-dependent coagulation assay. In some variations, the fIX activity is enhanced approximately between 5 fold and up to approximately 10 fold and up to or over approximately 20 fold.

Disclosed herein are modified fIX polypeptides containing an amino acid replacement in the fIX polypeptide, which may be an unmodified fIX polypeptide, wherein the amino acid replacement can be one or more of the following, wherein the dual sequence numbering is due to the fact that the fIX polypeptide contains a signal peptide and propeptide that comprises the first 46 amino acids, the first number represents the replacement with the signal peptide and the second number represents the replacement without the signal peptide: EGF2 (V132A-V86A); N313S-N267S; E323K-E277K; V326T-V280T; D338N-D292N; K339R-K293R; H361N-H315N; K362R-K316R; L367S-L321S; L366S-L320S; V368I-V322I; V367I-V321I; F399Y-F353Y; R404K-R358K; D232N-D186N; V243L-V197L; V248I-V242I; V257I-V211I; I262V-I216V; V269I-V223I; T271P-T225P; E286K-E240K; H289P-H243P; I299V-I253V; A308T-A262T; R384E-R338E or R384L-R338L.

Disclosed herein are modified fIX polypeptides containing an amino acid replacement in an unmodified fIX polypeptide, wherein the amino acid replacement can be one or more of the following, wherein the dual sequence numbering is due to the fact that the fIX polypeptide contains a signal peptide and propeptide that comprises the first 46 amino acids, the first number represents the replacement position in a polypeptide including the signal peptide and the second number represents the replacement in a polypeptide without the signal peptide: EGF2 (V132A-V86A); N313S-N267S; E323K-E277K; V326T-V280T; D338N-D292N; K339R-K293R; H361N-H315N; K362R-K316R; L367S-L321S; L366S-L320S; V368I-V322I; V367I-V321I; F399Y-F353Y; R404K-R358K; D232N-D186N; V243L-V197L;V248I-V242I; V257I-V211I; I262V-I216V; V269I-V223I; T271P-T225P; E286K-E240K; H289P-H243P; I299V-I253V; A308T-A262T; R384E-R338E or R384L-338L.

In some embodiments, an isolated nucleic acid molecule is provided that comprises a nucleic acid sequence encoding a fIX protein comprising an amino acid sequence at least 95% identical to human fIX and further includes an amino acid replacement, wherein the amino acid replacement can be one or more of the following: EGF2 (V132A-V86A); N313S-N267S; E323K-E277K; V326T-V280T; D338N-D292N; K339R-K293R; H361N-H315N; K362R-K316R; L367S-L321S; L366S-L320S; V368I-V3221; V367I-V321I; F399Y-F353Y; R404K-R358K; D232N-D186N; V243L-V197L;V248I-V242I; V257I-V211I; I262V-I216V; V269I-V223I; T271P-T225P; E286K-E240K; H289P-H243P; I299V-I253V; A308T-A262T; R384E-R338E or R384L-R338L.

In some embodiments, an isolated nucleic acid molecule is provided that comprises a nucleic acid sequence encoding a fIX protein comprising an amino acid sequence at least 95% identical to human fIX, e.g., SEQ ID No. 1 and further includes an amino acid replacement, wherein the amino acid replacement can be one or more of the following EGF2 (V132A-V86A); N313S-N267S; E323K-E277K; V326T-V280T; D338N-D292N; K339R-K293R; H361N-H315N; K362R-K316R; L367S-L321S; L366S-L320S; V368I-V322I; V367I-V321I; F399Y-F353Y; R404K-R358K; D232N-D186N; V243L-V197L; V248I-V242I; V257I-V211I; I262V-I216V; V269I-V223I; T271P-T225P; E286K-E240K; H289P-H243P; I299V-I253 V; A308T-A262T; R384E-R338E or R384L- R338L.

In some embodiments, an isolated nucleic acid molecule is provided that comprises a nucleic acid sequence encoding a fIX protein comprising an amino acid sequence at least 95% identical to human fix, e.g., SEQ ID No. 1 and further includes an amino acid replacement, wherein the amino acid replacements are: D338N-D292N and L367S-L321S. See, for example, SEQ ID No. 52, which is referred to herein as variation “Alpha.”

In some embodiments, an isolated nucleic acid molecule is provided that comprises a nucleic acid sequence encoding a fix protein comprising an amino acid sequence at least 95% identical to human fIX, e.g., SEQ ID No. 1 and further includes an amino acid replacement, wherein the amino acid replacements are: V132A-V86A; D338N-D292N; K362R-K316R; and L367S-L321S. See, for example, SEQ ID No. 53, which is referred to herein as variation “Beta.”

In some embodiments, an isolated nucleic acid molecule is provided that comprises a nucleic acid sequence encoding a fIX protein comprising an amino acid sequence at least 95% identical to human fIX, e.g., SEQ ID No. 1 and further includes an amino acid replacement, wherein the amino acid replacements are: V132A-V86A; E323K-E277K; V326T-V280T; D338N-D292N; K339R- K293R; K362R-K316R; and L367S-L321S. See, for example, SEQ ID No. 54, which is referred to herein as variation “Delta.”

In some embodiments, an isolated nucleic acid molecule is provided that comprises a nucleic acid sequence encoding a fix protein comprising an amino acid sequence at least 95% identical to human fIX, e.g., SEQ ID No. 1 and further includes an amino acid replacement, wherein the amino acid replacements are: V132A-V86A; E323K-E277K; D338N-D292N; K362R-K316R; and L367S-L321S. See, for example, SEQ ID No. 55, which is referred to herein as variation “Gamma ”

In some embodiments, an isolated nucleic acid molecule is provided that comprises a nucleic acid sequence encoding a fIX protein comprising an amino acid sequence at least 95% identical to human fIX, e.g., SEQ ID No. 1 and further includes an amino acid replacement, wherein the amino acid replacements are: V132A-V86A; E323K-E277K; D338N-D292N; K362R-K316R; L367S-L321S; and V132A-V86A. See, for example, SEQ ID No. 56, which is referred to herein as variation “Gamma (with).”

In some embodiments, an isolated nucleic acid molecule is provided that comprises a nucleic acid sequence encoding a fIX protein comprising an amino acid sequence at least 95% identical to human fIX, e.g., SEQ ID No. 1 and further includes an amino acid replacement, wherein the amino acid replacements are: V132A-V86A; D338N-D292N; K362R-K316R; L367S-L321S; and V132A-V86A. See, for example, SEQ ID No. 57, which is referred to herein as variation “Beta (with).”

Also provided are vectors, such as an adeno-associated virus (AAV) vector, containing the nucleic acid molecules, as well as isolated fIX proteins encoded by the nucleic acid molecules.

In some embodiments, a method of inducing blood clotting in a subject in need thereof is provided. The method comprises administering to the subject a therapeutically effective amount of a vector (such as an AAV vector) encoding a recombinant clotting factor as described herein. In some embodiments, t63/107he subject is a subject with a clotting disorder, such as hemophilia A or hemophilia B. In some embodiments, the clotting disorder is hemophilia B and the subject is administered a vector comprising a nucleic acid molecule encoding a recombinant fIX protein.

The foregoing and other features and advantages of this disclosure will become more apparent from the following detailed description of several embodiments which proceeds with reference to the accompanying figures.

### BRIEF DESCRIPTION OF THE FIGURES

FIGS. 1A and 1B illustrate a sequence alignment of the human fIX (SEQ ID NO: 1) and a variant thereof, including An96 fIX (SEQ ID NO: 2), and the amino acid sequences coded by hfIX-96sp3pro (SEQ ID NO: 3), or An96-hfIXpro (SEQ ID NO: 4), or hfIX-96appro (SEQ ID NO: 5), or hfIX-96e2pro (SEQ ID NO: 6), or hfIX-96e2V86A (SEQ ID NO: 7), or hfIX-96e2V86Apro2 (SEQ ID NO: 8), or hfIX-96ge2appro (SEQ ID NO: 9), or hfIX-96ge2pro (SEQ ID NO: 10), or hfIX-96ge2pro2 (SEQ ID NO: 11), or hfIX-96gpro (SEQ ID NO: 12), or hfIX-96pro (SEQ ID NO: 13), or hfIX-96pro2 (SEQ ID NO: 14).

FIG. 2 shows fIX activity levels of various fIX variants expressed in cells using AAV vectors encoding the indicated fIX variants.

FIG. 3 shows in vivo data for fIX activity levels in serum of fIX deficient mice treated with AAV vectors encoding the indicated fIX variants.

FIG. 4 shows in vivo data for fIX activity levels in serum of fIX^{+/+} mice treated with AAV2/8 vectors containing a liver-directed promoter (HCB) and encoding the indicated fIX variants. The change in fIX activity level pre- and post-AAV administration is plotted. Statistical comparisons were made by one-way ANOVA and Holm-Sidak post-hoc analysis. Asterisks denote P<0.05.

FIG. 5 shows fVIII activity levels of various fVIII variants expressed in HEK293T17 cells using plasmid DNA expression vectors encoding the indicated fVIII variants.

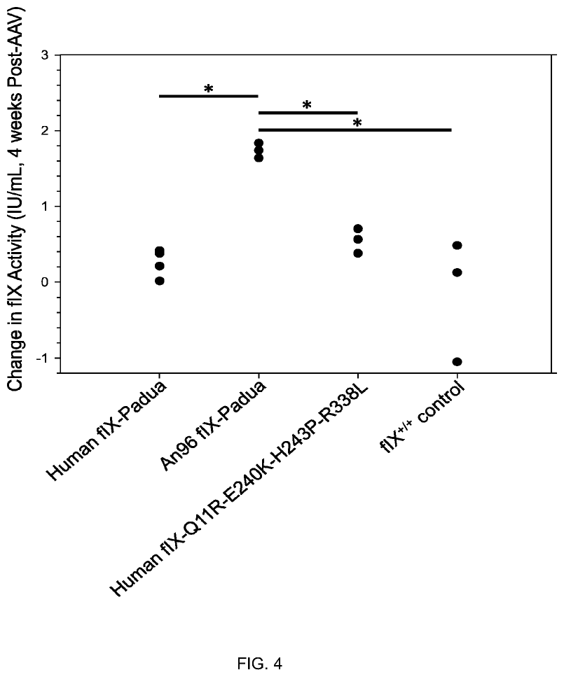

FIG. 6 shows the elements and structural features of fIX.

FIG. 7 shows fIX activity levels of various fIX variants expressed in Huh-7cells using AAV vectors encoding the indicated fIX variants.

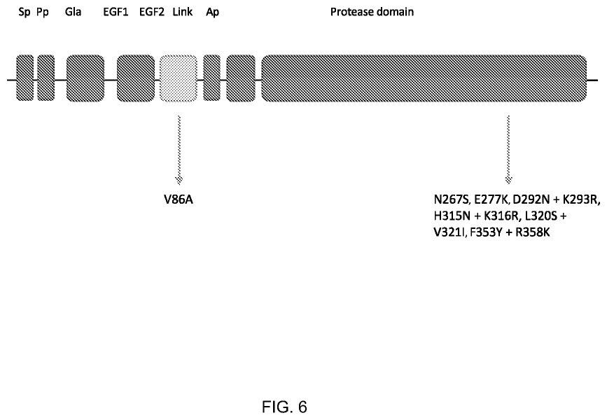

FIG. 8 shows hfIX 96wt hybrid Plasma activity (U/mL) against Time (Weeks).

FIG. 9 shows plotted data of the gene therapy phenotypic correction 96 wt Hu hybrid cohort.

FIG. 10 shows an amino acid sequence alignment between hflX and An96 proteins.

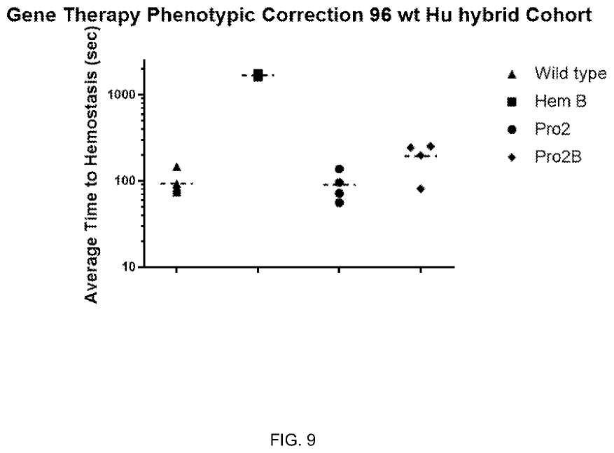

FIG. 11 shows an amino acid sequence alignment between hfIX and An96 mature secreted proteins.

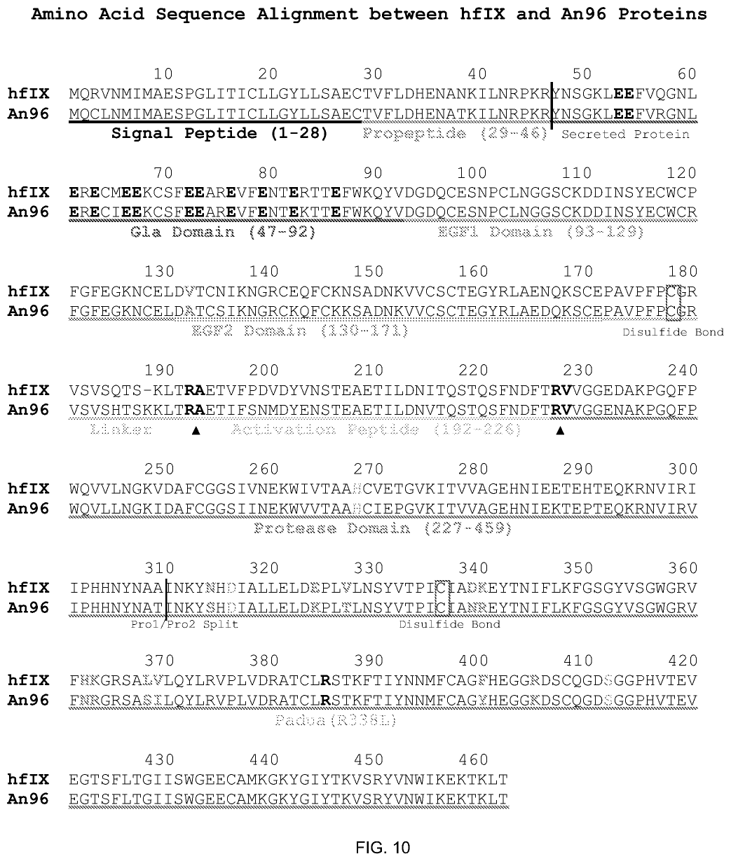

FIG. 12 shows a construct map of human fIX (SEQ ID NO: 1) and a variant thereof, including amino acids encoded by An96 fIX (SEQ ID NO: 2), hfIX-96sp3pro (SEQ ID NO: 3), or An96-hfIXpro (SEQ ID NO: 4), or hfIX-96appro (SEQ ID NO: 5), or hfIX-96e2pro (SEQ ID NO: 6), or hflX-96e2V86A (SEQ ID NO: 7), or hfIX-96e2V86Apro2 (SEQ ID NO: 8), or hfIX-96ge2appro (SEQ ID NO: 9), or hfIX-96ge2pro (SEQ ID NO: 10), or hfIX-96ge2pro2 (SEQ ID NO: 11), or hfIX-96gpro (SEQ ID NO: 12), or hfIX-96pro (SEQ ID NO: 13), or hfIX-96pro2 (SEQ ID NO: 14).

FIG. 13 shows fIX activity of various constructs disclosed herein against hfIX.

FIG. 14 shows fIX activity of various constructs disclosed herein against hfIX.

FIG. 15A shows relative fIX expression of various constructs disclosed herein.

FIG. 15B shows relative fIX expression of various constructs disclosed herein.

FIG. 16 shows validation of fIX activity of the fIX constructs disclosed herein versus fIX Padua and An96.

FIG. 17 shows validation of fIX activity in an AAV2/8 system hemophilia B mice.

FIG. 18 shows a comparison of fibrin clot formation and specific activity of fIX variants.

FIG. 19 shows enzyme kinetics of various fIX variants.

FIG. 20 shows fIX activity of various fIX variants.

FIG. 21 shows relative fIX activity of various fIX constructs with domain substitutions.

FIG. 22 shows relative fIX activity of various fIX constructs with amino acid substitutions.

FIG. 23 shows relative fIX activity of various fIX constructs.

FIG. 24 shows relative fIX activity of various fIX constructs.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

### DETAILED DESCRIPTION

### I. Terms

Unless otherwise noted, technical terms are used according to conventional usage. Definitions of common terms in molecular biology may be found in Krebs et al. (eds.), Lewin's genes XII, published by Jones & Bartlett Learning, 2017; Kendrew et al. (eds.), The Encyclopedia of Molecular Biology, published by Blackwell Science Ltd., 2009 (ISBN 9780632021826). The singular terms “a,” “an,” and “the” include plural referents unless context clearly indicates otherwise. “Comprising A or B” means including A, or B, or A and B. Although methods and materials similar or equivalent to those described herein can be used in the practice or testing of the present disclosure, suitable methods and materials are described below. In addition, the materials, methods, and examples are illustrative only and not intended to be limiting. In case of conflict, the present specification, including explanations of terms, will control. In order to facilitate review of the various embodiments of the disclosure, the following explanations of specific terms are provided:

5′ and/or 3′: Nucleic acid molecules (such as, DNA and RNA) are said to have “5′ ends” and “3′ ends” because mononucleotides are reacted to make polynucleotides in a manner such that the 5′ phosphate of one mononucleotide pentose ring is attached to the 3′ oxygen of its neighbor in one direction via a phosphodiester linkage. Therefore, one end of a linear polynucleotide is referred to as the “5′ end” when its 5′ phosphate is not linked to the 3′ oxygen of a mononucleotide pentose ring. The other end of a polynucleotide is referred to as the “3′ end” when its 3′ oxygen is not linked to a 5′ phosphate of another mononucleotide pentose ring. Notwithstanding that a 5′ phosphate of one mononucleotide pentose ring is attached to the 3′ oxygen of its neighbor, an internal nucleic acid sequence also may be said to have 5′ and 3′ ends.

In either a linear or circular nucleic acid molecule, discrete internal elements are referred to as being “upstream” or 5′ of the “downstream” or 3′ elements. With regard to DNA, this terminology reflects that transcription proceeds in a 5′ to 3′ direction along a DNA strand. Promoter and enhancer elements, which direct transcription of a linked gene, are generally located 5′ or upstream of the coding region. However, enhancer elements can exert their effect even when located 3′ of the promoter element and the coding region. Transcription termination and polyadenylation signals are located 3′ or downstream of the coding region.

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 11 recognized serotypes of AAV (AAV1-11).

Administration/Administer: To provide or give a subject an agent, such as a therapeutic agent (e.g. a recombinant AAV), by any effective route. Exemplary routes of administration include, but are not limited to, injection (such as subcutaneous, intramuscular, intradermal, intraperitoneal, and intravenous), oral, intraductal, sublingual, rectal, transdermal, intranasal, vaginal and inhalation routes.

Bleeding Time Assay: An assay used to measure the amount of time it takes for a subject's blood to clot. A blood pressure cuff is placed on the upper arm and inflated. Two incisions are made on the lower arm. These are about 10 mm (less than ½ inch) long and 1 mm deep (just deep enough to cause minimal bleeding). The blood pressure cuff is immediately deflated. Blotting paper is touched to the cuts every 30 seconds until the bleeding stops. The length of time it takes for the cuts to stop bleeding is recorded. In normal, non-hemophiliacs, bleeding stops within about one to ten minutes and may vary from lab to lab, depending on how the assay is measured. In contrast, severe hemophiliacs having less than 1% of normal levels of the appropriate clotting factor have a whole blood clotting time of greater than 60 minutes. In mice, the bleeding time is assayed by transecting the tip of the tail and periodically touching a blotting paper until a clot is formed at the tip of the tail. Normal bleeding time is between 2-4 minutes. In contrast, hemophiliac mice having less than 1% of normal levels of the appropriate clotting factor have a bleeding time of greater than 15 minutes.

cDNA (complementary DNA): A piece of DNA lacking internal, non-coding segments (introns) and regulatory sequences that determine transcription. cDNA is synthesized in the laboratory by reverse transcription from messenger RNA extracted from cells. cDNA can also contain untranslated regions (UTRs) that are responsible for translational control in the corresponding RNA molecule.

Clotting disorder: A general term for a wide range of medical problems that lead to poor blood clotting and continuous bleeding. Doctors also refer to clotting disorders by terms such as, for example, coagulopathy, abnormal bleeding and bleeding disorders. Clotting disorders include any congenital, acquired or induced defect that results in abnormal (or pathological) bleeding. Examples include, but are not limited to, disorders of insufficient clotting or hemostasis, such as hemophilia A (a deficiency in fVIII), hemophilia B (a deficiency in fIX), hemophilia C (a deficiency in Factor XI), proconvertin deficiency (a deficiency in fVII), abnormal levels of clotting factor inhibitors, platelet disorders, thrombocytopenia, vitamin K deficiency and von Willebrand's disease.

Some clotting disorders are present at birth and in some instances are inherited disorders. Specific examples include, but are not limited to: hemophilia A, hemophilia B, protein C deficiency, and Von Willebrand's disease. Some clotting disorders are developed during certain illnesses (such as vitamin K deficiency, severe liver disease), or treatments (such as use of anticoagulant drugs or prolonged use of antibiotics).

Clotting Factor VII (fVII): fVII is a vitamin K-dependent zymogen protein required for the efficient clotting of blood. When combined with tissue factor, fVII becomes proteolytically activated (fVIIa) and functions in coagulation as an activator of factor IX and factor X. At suprapyhsiologic levels, fVIIa can display tissue factor independent procoagulant activity as well. A concentration of about 0.5 μg/ml of fVII in the blood is considered normal. Deficiency of fVII is associated with congenital proconvertin deficiency, which presents as a hemophilia-like bleeding disorder. fVII is biosynthesized as a single-chain zymogen containing a domain structure with an N-terminal signal peptide (approximately residues −20 to −1), a γ-carboxyglutamic acid (Gla) rich domain (approximately residues 1-63), two epidermal growth factor (EGF)-like domains (approximately residues 64-100 [EGF1] and 101-170 [EGF2]), and a latent C-terminal serine protease domain (approximately residues 171-444). For activation, fVII requires a single peptide bond cleavage at Arg190-Iso191. This results in the formation of fVIIa consisting of a light chain composed of the Gla, EGF1, and EGF2 domains linked through a single disulphide bond to a heavy chain containing the protease domain. A substantial amount of information is available on the structure and function of fVII protein; see, e.g., Vadivel et al. “Structure and function of Vitamin K-dependent coagulant and anticoagulant proteins.” in Hemostasis and Thrombosis-Basic Principles and Clinical Practice. 6^{th} edition. Marder et al. (Eds.). Philadelphia: Lippincott Williams and Wilkens, 2013. Pages 208-232, which is incorporated by reference herein in its entirety. fVII nucleic acid and protein sequences are publicly available (for example see UniProtKB/Swiss-Prot Ref. No. P08709.1). fVII variants are provided herein that have increased fVII activity for blood clotting.

Clotting Factor VIII (fVIII): fVIII is a protein required for the efficient clotting of blood, and functions in coagulation as a cofactor in the activation of factor X by fix. FVIII contains multiple domains (A1-A2-B-ap-A3-C1-C2) and circulates in blood in an inactivated form bound to von Willebrand factor (VWF). Thrombin cleaves fVIII causing dissociation with VWF ultimately leading to fibrin formation through fix. Congenital hemophilia A is associated with genetic mutations in the fVIII gene and results in impaired clotting due to lower than normal levels of circulating fVIII. A concentration of about 100 ng/ml for fVIII in the blood is considered in the normal range. Severe forms of hemophilia A can result when a patient has less than about 1% of the normal amount of fVIII (i.e. less than about 1 ng of fVIII per ml of blood). fVIII is synthesized as an approximate 2351 amino acid single chain precursor protein, which is proteolytically processed. The human factor VIII gene (186,000 base-pairs) consists of 26 exons ranging in size from 69 to 3,106 bp and introns as large as 32.4 kilobases (kb). Examples of fVIII nucleic acid and protein sequences are publicly available (for example, see Genbank Accession Nos: K01740, M14113, and E00527). fVIII variants are provided herein that have increased fVIII activity for blood clotting but are reduced in size, such as fVIII variants that lack the fVIII B domain and also have one or more amino acid variations that provide for increased fVIII activity.

Clotting Factor IX (fIX): fIX is a vitamin K-dependent protein required for the efficient clotting of blood, and functions in coagulation as an activator of factor X. A concentration of about 1-5 μg/ml of fIX in the blood is considered in the normal range. Deficiency of fIX is associated with hemophilia B, and severe cases result when the concentration of fIX is less than about 1% of the normal concentration of fIX (i.e. less than about 0.01-0.05 μg fIX per ml of blood). fIX is biosynthesized as a single-chain zymogen containing a domain structure with an N-terminal signal peptide (approximately residues −28 to −1), a γ-carboxyglutamic acid (Gla) rich domain (approximately residues 1-40), a short hydrophobic segment (approximately residues 41-46), two epidermal growth factor (EGF)-like domains (approximately residues 47-84 [EGF1] and 85-127[EGF2]), an activation peptide (approximately residues 146-180), and a latent C-terminal serine protease domain (approximately residues 181-415). For activation, fIX requires two peptide bond cleavages, one at Arg145-Ala146 and one at Arg180-Va1181, releasing a 35-residue activation peptide. This results in the formation of activated fIX (fIXa) consisting of a light chain composed of the Gla, EGF1, and EGF2 domains linked through a single disulphide bond to a heavy chain containing the protease domain (185-415). A substantial amount of information is available on the structure and function of fIX protein; see, e.g., Vadivel et al. “Structure and function of Vitamin K-dependent coagulant and anticoagulant proteins.” in Hemostasis and Thrombosis-Basic Principles and Clinical Practice. 6^{th} edition. Marder et al. (Eds.). Philadelphia: Lippincott Williams and Wilkens, 2013. Pages 208-232, which is incorporated by reference herein in its entirety. fIX nucleic acid and protein sequences are publicly available (see for example UniProtKB/Swiss-Prot Ref. No. P00740.2. Factor IX variants are provided herein that have increased fIX activity for blood clotting.

Codon-optimized: A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

The term “liver specific amino acids codons” refers to codons that are differentially utilized-represented in genes highly expressed within the human liver compared to the codon usage of the entire coding region of the human genome. A liver-codon optimization strategy uses a maximum amount of liver specific amino acid codons seeks to avoid codons that are under-represented, e.g., because of low quantities of codon matching tRNA in liver cells resulting in slower protein translation.

Control: A reference standard. In some embodiments, the control is a negative control sample obtained from a healthy patient. In other embodiments, the control is a positive control sample obtained from a patient diagnosed with hemophilia. In still other embodiments, the control is a historical control or standard reference value or range of values (such as a previously tested control sample, such as a group of hemophilia A patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine (A), guanine (G), cytosine (C), and thymine (T) bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a specific protein, or a fragment thereof, encompasses both the sense strand and its reverse complement. For instance, it is appropriate to generate probes or primers from the reverse complement sequence of the disclosed nucleic acid molecules.

Expression: Transcription or translation of a nucleic acid sequence. For example, an encoding nucleic acid sequence (such as a gene) can be expressed when its DNA is transcribed into RNA or an RNA fragment, which in some examples is processed to become mRNA. An encoding nucleic acid sequence (such as a gene) may also be expressed when its mRNA is translated into an amino acid sequence, such as a protein or a protein fragment. In a particular example, a heterologous gene is expressed when it is transcribed into RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcriptional terminators, a start codon (ATG) in front of a protein-encoding gene, splice signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Gene: A nucleic acid sequence, typically a DNA sequence, that comprises control and coding sequences necessary for the transcription of an RNA, whether an mRNA or otherwise. For instance, a gene may comprise a promoter, one or more enhancers or silencers, a nucleic acid sequence that encodes a RNA and/or a polypeptide, downstream regulatory sequences and, possibly, other nucleic acid sequences involved in regulation of the expression of an mRNA.

As is well known in the art, most eukaryotic genes contain both exons and introns. The term “exon” refers to a nucleic acid sequence found in genomic DNA that is bioinformatically predicted and/or experimentally confirmed to contribute a contiguous sequence to a mature mRNA transcript. The term “intron” refers to a nucleic acid sequence found in genomic DNA that is predicted and/or confirmed not to contribute to a mature mRNA transcript, but rather to be “spliced out” during processing of the transcript.

Gene therapy: The introduction of a heterologous nucleic acid molecule into one or more recipient cells, wherein expression of the heterologous nucleic acid in the recipient cell affects the cell's function and results in a therapeutic effect in a subject. For example, the heterologous nucleic acid molecule may encode a protein, which affects a function of the recipient cell.

Hemophilia: A blood coagulation disorder caused by a deficient clotting factor activity, which decreases hemostasis. Severe forms result when the concentration of clotting factor is less than about 1% of the normal concentration of the clotting factor in a normal subject. In some subjects, hemophilia is due to a genetic mutation which results in impaired expression of a clotting factor. In others, hemophilia is an auto-immune disorder, referred to as acquired hemophilia, in which the antibodies which are generated against a clotting factor in a subject result in decreased hemostasis.

Hemophilia A results from a deficiency of functional clotting fVIII, while hemophilia B results from a deficiency of functional clotting fIX. These conditions which are due to a genetic mutation are caused by an inherited sex-linked recessive trait with the defective gene located on the X chromosome, and this disease is therefore generally found only in males. The severity of symptoms can vary with this disease, and the severe forms become apparent early on. Bleeding is the hallmark of the disease and typically occurs when a male infant is circumcised. Additional bleeding manifestations make their appearance when the infant becomes mobile. Mild cases may go unnoticed until later in life when they occur in response to surgery or trauma. Internal bleeding may happen anywhere, and bleeding into joints is common.

Hemostasis: Arrest of bleeding blood by blood clot formation. Blood clotting time is the length of time it takes for peripheral blood to clot using an activated partial thromboplastin time assay (APTT) or by measuring bleeding time. In a particular embodiment, the blood clotting time decreases by at least 50%, for example at least 60%, at least 70%, at least 75%, at least 80%, at least 90%, at least 95%, at least 98%, at least 99% or even about 100% (i.e. the blood clotting time is similar to what is observed for a normal subject) when compared to the blood clotting time of the subject prior to administration of a therapeutic vector encoding the appropriate clotting factor as described herein. In yet another embodiment, the blood clotting time in the affected subject is corrected to about 50% of a normal subject, to about 75% of a normal subject, to about 90% of a normal subject, for example to about 95%, for example about 100%, after oral administration of a therapeutically effective amount of the appropriate clotting factor. As used herein, “about” refers to plus or minus 5% from a reference value. Thus, about 50% refers to 47.5% to 52.5%.

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed vectors.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as vector compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Purified: The term purified does not require absolute purity; rather, it is intended as a relative term. Thus, for example, a purified protein preparation is one in which the protein (such as a fVII, fVIII, or fIX protein) is more enriched than the peptide or protein is in its natural environment within a cell. In one embodiment, a preparation is purified such that the protein represents at least 50% of the total protein content of the preparation.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Preventing, treating or ameliorating a disease: “Preventing” a disease (such as hemophilia) refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Promoter: A region of DNA that directs/initiates transcription of a nucleic acid (e.g. a gene). A promoter includes necessary nucleic acid sequences near the start site of transcription. Typically, promoters are located near the genes they transcribe. A promoter also optionally includes distal enhancer or repressor elements which can be located as much as several thousand base pairs from the start site of transcription. A tissue-specific promoter is a promoter that directs/initiated transcription primarily in a single type of tissue or cell. For example, a liver-specific promoter is a promoter that directs/initiates transcription in liver tissue to a substantially greater extent than other tissue types.

Protein: A biological molecule expressed by a gene or other encoding nucleic acid (e.g., a cDNA) and comprised of amino acids.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid molecule is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques.

A recombinant virus is one that includes a genome that includes a recombinant nucleic acid molecule. As used herein, “recombinant AAV” refers to an AAV particle in which a recombinant nucleic acid molecule (such as a recombinant nucleic acid molecule encoding a clotting factor) has been packaged.

A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell, or into the genome of a recombinant virus.

Sequence identity: The identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

As used herein, reference to “at least 90% identity” refers to “at least 90%, at least 91%, at least 92%, at least 93%, at least 94%, at least 95%, at least 96%, at least 97%, at least 98%, at least 99%, or even 100% identity” to a specified reference sequence.

Subject: Living multi-cellular vertebrate organisms, a category that includes human and non-human mammals.

Therapeutically effective amount: The amount of agent, such as a disclosed viral vector encoding a clotting factor, that is sufficient to prevent, treat (including prophylaxis), reduce and/or ameliorate the symptoms and/or underlying causes of a disorder or disease, for example to prevent, inhibit, and/or treat hemophilia. For example, this can be the amount of a recombinant viral vector encoding a novel clotting factor as described herein that produces sufficient amounts of the clotting factor to decrease the time it takes for the blood of a subject to clot. For example, this can be the amount of a recombinant AAV vector encoding a novel clotting factor as described herein that produces sufficient amounts of the clotting factor to decrease the time it takes for the blood of a subject to clot. In some embodiments, the vector is a gamma-retroviral vector, a lentiviral vector, or an adenoviral vector.

In one example, a desired response is to reduce clotting time in a subject (such as a subject with hemophilia), for example as measured using a bleeding time assay. The clotting time does not need to be completely restored to that of normal healthy subjects without hemophilia for the method to be effective. For example, administration of a therapeutically effective amount of a vector (such as a fIX encoding vector) as disclosed herein can decrease the clotting time (or other symptom of the hemophilia) by a desired amount, for example by at least 20%, at least 30%, at least 40%, at least 50%, at least 60%, at least 70%, at least 80%, at least 90%, at least 95%, at least 98%, at least 100% or more, as compared to a suitable control.

It is understood that to obtain a therapeutic response to the disease or condition can require multiple administrations of a therapeutic agent. Thus, a therapeutically effective amount encompasses a fractional dose that contributes in combination with previous or subsequent administrations to attaining a therapeutic outcome in the patient. For example, a therapeutically effective amount of an agent can be administered in a single dose, or in several doses, for example daily, during a course of treatment. However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an adeno-associated virus (AAV) vector. In some embodiments, the vector is a gamma-retroviral vector, a lentiviral vector, or an adenoviral vector.

II. Novel Clotting factors

The blood clotting system is a proteolytic cascade. Blood clotting factors are present in the plasma as a zymogen, an inactive form, which on activation undergoes proteolytic cleavage to release the active factor form the precursor molecule. The ultimate goal is to produce thrombin. Thrombin converts fibrinogen into fibrin, which forms a clot.

Factor X is the first molecule of the common pathway and is activated by a complex of molecules containing activated fix, fVIII, calcium, and phospholipids which are on the platelet surface. FVIII is activated by thrombin, and it facilitates the activation of factor X by fIXa. Congenital hemophilia A is associated with genetic mutations in the fVIII gene and results in impaired clotting due to lower than normal levels of circulating fVIII. Hemophilia B is similarly associated with genetic mutations in the fIX gene. Proconvertin deficiency is similarly associated with mutations in the fVII gene.

As discussed in Example 1, novel fIX sequences were identified from corresponding ancestral variants and assessed for clotting factor activity. The identified sequences, disclosed and claimed herein, provide for increased clotting factor activity relative to the corresponding human clotting factor.

In some embodiments, a nucleic acid molecule is provided that encodes a protein with fIX activity comprising an amino acid sequence set forth as residues An96 fIX (SEQ ID NO: 2), Human fIX (SEQ ID NO: 1), Human fIX (SEQ ID NO: 15), any other fIX known or disclosed herein (e.g., SEQ ID NO: 16, SEW ID NO: 18, SEQ ID NOS: 19-26, SEQ ID NOS: 52-57, or an amino acid sequence at least 95%, at least 96%, at least 97%, at least 98%, or at least 99% identical thereto. Residues ˜1-46 of SEQ ID NO: 1 (Human fIX) are the fIX signal peptide and propeptide. Similarly, Residues ˜1-46 of SEQ ID NO: 2 (AN96 fIX) are the fIX signal peptide and propeptide. In some embodiments, a nucleic acid molecule is provided that encodes a protein with fIX activity comprising an amino acid sequence set forth as SEQ ID NO: 2 (An96 fIX with signal peptide and propeptide), or an amino acid sequence at least 95%, at least 96%, at least 97%, at least 98%, or at least 99% identical thereto. In some embodiments, a nucleic acid molecule is provided that encodes a protein with fIX activity comprising an amino acid sequence set forth as SEQ ID NO: 16 (An96 fIX Padua with signal peptide and propeptide), or an amino acid sequence at least 95%, at least 96%, at least 97%, at least 98%, or at least 99% identical thereto. In some embodiments, a nucleic acid molecule is provided that encodes a protein with fIX activity comprising an amino acid sequence set forth as SEQ ID NO: 52 (An96 fIX Alpha with signal peptide and propeptide), or an amino acid sequence at least 95%, at least 96%, at least 97%, at least 98%, or at least 99% identical thereto. In some embodiments, a nucleic acid molecule is provided that encodes a protein with fIX activity comprising an amino acid sequence set forth as SEQ ID NO: 53 (An96 fIX Beta with signal peptide and propeptide), or an amino acid sequence at least 95%, at least 96%, at least 97%, at least 98%, or at least 99% identical thereto. In some embodiments, a nucleic acid molecule is provided that encodes a protein with fIX activity comprising an amino acid sequence set forth as SEQ ID NO: 54 (An96 fIX Delta with signal peptide and propeptide), or an amino acid sequence at least 95%, at least 96%, at least 97%, at least 98%, or at least 99% identical thereto. In some embodiments, a nucleic acid molecule is provided that encodes a protein with fIX activity comprising an amino acid sequence set forth as SEQ ID NO: 55 (An96 fIX Gamma with signal peptide and propeptide), or an amino acid sequence at least 95%, at least 96%, at least 97%, at least 98%, or at least 99% identical thereto. In some embodiments, a nucleic acid molecule is provided that encodes a protein with fIX activity comprising an amino acid sequence set forth as SEQ ID NO: 56 (An96 fIX Gamma with Padua mutation and with signal peptide and propeptide), or an amino acid sequence at least 95%, at least 96%, at least 97%, at least 98%, or at least 99% identical thereto. In some embodiments, a nucleic acid molecule is provided that encodes a protein with fIX activity comprising an amino acid sequence set forth as SEQ ID NO: 57 (An96 fIX Beta with Padua mutation and with signal peptide and propeptide), or an amino acid sequence at least 95%, at least 96%, at least 97%, at least 98%, or at least 99% identical thereto. In alternative embodiments, a different signal peptide and/or propeptide can be used in place of the signal peptide and/or propeptide of SEQ ID NO: 2 and/or SEQ ID NO: 16, such as an IL2 signal peptide and/or factor X propeptide. In some embodiments, a nucleic acid molecule is provided that encodes a protein with fIX activity comprising an amino acid sequence set forth as SEQ ID NO: 15 (human fIX without signal peptide and propeptide), or an amino acid sequence at least 95%, at least 96%, at least 97%, at least 98%, or at least 99% identical thereto. Any of SEQ ID NOS: 52-57 and SEQ ID NOS: 18-26 may be modified by removing the signal peptide and propeptide as discussed above, e.g., removing the nucleic acids that code for Residues ˜1-46 of the respective amino acid sequence.

Substitutions are based on ancestral fIX sequences. Ancestral fIX sequences were identified through ASR and synthesized de novo for in vitro expression studies. Ancestral sequence 96 (An96) was identified to have greater activity than human fIX and comparable activity to fIX-Padua (R338L). An96 is 90% human at the amino acid level and through domain swapping studies between human fIX and An96, domains that confer greater activity were identified to be the EGF2 and protease domains of An96. Briefly, constructs were generated by domain swapping and cloned into AAV2 expression plasmids. Huh-7 liver cells were transiently transfected with each construct, and expression of fIX measured from conditioned medium by one-stage APTT-dependent clotting assay. Activity was normalized to hflX expression levels and compared to An96 expression levels. In a series of reiterative experiments, the EGF2 and protease domains of An96 were identified to confer enhanced levels of fIX expression when substituted into hfIX. Through amino acid substitution studies, the V132A-V86A single point mutation in the EGF2 domain and the N313S-N267S, E323K-E277K, D338N-D292N, K339R-K293R, H361N-H315N, K362R-K316R, L366S-L320S, F399Y-F353Y, R404K-R358K mutations in the protease domain confer ˜10-fold fIX activity compared to human fIX equivalent to that of An96. Through further narrowing amino acid substitution studies, the V132A-V86A single point mutation in the EGF2 domain and the E323K-E277K, D338N-D292N, K339R-K293R, K362R-K316R, L367S-L321S mutations in the protease domain were found to confer ˜10-fold fIX activity compared to human fIX equivalent and to or surpassing that of An96. Through further narrowing amino acid substitution studies, the V132A-V86A single point mutation in the EGF2 domain, several exemplary amino acid sequences were made, tested, and found to confer ˜10-fold fIX activity compared to human fIX equivalent to or surpassing that of An96. The amino acid sequences for these constructs are found at SED ID NO: 52, fIX Optimized sequence Alpha, having V132A-V86A single point mutation in the EGF2 domain, D338N-D292N, and L367S-L321S; SEQ ID NO: 53, fIX Optimized sequence Beta, having V86A single point mutation in the EGF2 domain, D338N-D292N, K362R - K316R and L367S -L321S; SEQ ID NO: 54, fIX Optimized Sequence Delta having V132A-V86A single point mutation in the EGF2 domain, E323K-E277K, V326T-V280T, D338N-D292N, K339R-K293R, K362R-K316R and L367S-L321S; SEQ ID NO: 55, fIX Optimized sequence Gamma, having V132A-V86A single point mutation in the EGF2 domain, E323K-E277K, D338N-D292N, K362R-K316R and L367S-L321S; SEQ ID NO: 56, fIX Optimized sequence, Gamma (with Padua), having V132A-V86A single point mutation in the EGF2 domain, Padua, E323K-E277K, D338N-D292N, K362R-K316R and L367S-L321S; and SEQ ID NO: 57, fIX Optimized sequence Beta, having V86A single point mutation in the EGF2 domain, Padua, D338N-D292N, K362R-K316R and L367S-L321S. For each of the Optimized amino acid sequences, multiple codon-optimized nucleic acid sequences were devised and tested. For example, SEQ ID NOS: 28 through 31 are codon optimized nucleic acid sequences that code for fIX optimized sequence, Alpha, SEQ ID NO: 52; SEQ ID NOS: 32 through 35 are codon optimized nucleic acid sequences that code for fIX optimized sequence Beta, SEQ ID NO: 53; SEQ ID NOS: 36 through 39 are codon optimized nucleic acid sequences that code for fIX optimized sequence Delta, SEQ ID NO: 54; SEQ ID NOS: 40-43 are codon optimized nucleic acid sequences that code for fIX optimized sequence Gamma, SEQ ID NO: 55. The fIX transgene(s) are cloned into AAV-expression plasmid containing desired ITR and AAV is manufactured for liver-directed in vivo delivery.

As discussed in Example 1, the nucleotide sequence encoding the various fIX amino acid sequences disclosed herein, e.g., SEQ ID NOS: 2, 16-26, and 52-57, were codon-optimized for expression in human liver. An exemplary liver codon optimized An96 fIX Padua sequence is provided as SEQ ID NO: 17 and/or hfIX-96sp3pro (SEQ ID NO: 3), or An96-hfIXpro (SEQ ID NO: 4), or hfIIX-96appro (SEQ ID NO: 5), or hfIX-96e2pro (SEQ ID NO: 6), or hfIX-96e2V86A (SEQ ID NO: 7), or hfIX-96e2V86Apro2 (SEQ ID NO: 8), or hfIX-96ge2appro (SEQ ID NO: 9), or hfIX-96ge2pro (SEQ ID NO: 10), or hfIX-96ge2pro2 (SEQ ID NO: 11), or hflX-96gpro (SEQ ID NO: 12), or hfIX-96pro (SEQ ID NO: 13), or hfIX-96pro2 (SEQ ID NO: 14), or fIX optimized sequence Alpha 1 (SEQ ID NO: 28), or fIX optimized sequence Alpha 2 (SEQ ID NO: 29), or fIX optimized sequence Alpha 3 (SEQ ID NO: 30), or fIX optimized sequence Alpha 4 (SEQ ID NO: 31), or fIX optimized sequence Beta 1 (SEQ ID NO: 32), or fIX optimized sequence Beta 2 (SEQ ID NO: 33), or fIX optimized sequence Beta 3 (SEQ ID NO: 34), or fIX optimized sequence Beta 4 (SEQ ID NO: 35), or fIX optimized sequence Delta 1 (SEQ ID NO: 36), or fIX optimized sequence Delta 2 (SEQ ID NO: 37), or fIX optimized sequence Delta 3 (SEQ ID NO: 38), or fIX optimized sequence Delta 4 (SEQ ID NO: 39), or fIX optimized sequence Gamma 1 (SEQ ID NO: 40), or fIX optimized sequence Gamma 2 (SEQ ID NO: 41), or fIX optimized sequence Gamma 3 (SEQ ID NO: 42), or fIX optimized sequence Gamma 4 (SEQ ID NO: 43), or fIX optimized sequence Gamma 22, padua (SEQ ID NO: 44), or fIX optimized sequence Gamma 3 Padua (SEQ ID NO: 45), or fIX optimized sequence Gamma 87 Padua (SEQ ID NO: 46), or fIX optimized sequence Gamma 2 Padua (SEQ ID NO: 47), or fIX optimized sequence Beta 82 (SEQ ID NO: 48), of fIX optimized sequence Beta 81 Padua (SEQ ID No: 49) or fIX optimized sequence Beta 23 Padua (SEQ ID NO: 50), or fIX optimized sequence Beta 39 Padua (SEQ ID NO: 51). In some embodiments, a recombinant nucleic acid molecule is provided comprising the nucleotide sequence set forth as nucleotides 139-1389 of SEQ ID NO: 17 and/or An96 fIX (SEQ ID NO: 2), hfIX-96sp3pro (SEQ ID NO: 3), or An96-hfIXpro (SEQ ID NO: 4), or hfIX-96appro (SEQ ID NO: 5), or hfIX-96e2pro (SEQ ID NO: 6), or hflX-96e2V86A (SEQ ID NO: 7), or hfIX-96e2V86Apro2 (SEQ ID NO: 8), or hfIX-96ge2appro (SEQ ID NO: 9), or HFix-96ge2pro (SEQ ID NO: 10), or hfIX-96ge2pro2 (SEQ ID NO: 11), or hflX-96gpro (SEQ ID NO: 12), or HFix-96pro (SEQ ID NO: 13), or hfIX-96pro2 (SEQ ID NO: 14), or fIX optimized sequence Alpha 1 (SEQ ID NO: 28), or fIX optimized sequence Alpha 2 (SEQ ID NO: 29), or fIX optimized sequence Alpha 3 (SEQ ID NO: 30), or fIX optimized sequence Alpha 4 (SEQ ID NO: 31), or fIX optimized sequence Beta 1 (SEQ ID NO: 32), or fIX optimized sequence Beta 2 (SEQ ID NO: 33), or fIX optimized sequence Beta 3 (SEQ ID NO: 34), or fIX optimized sequence Beta 4 (SEQ ID NO: 35), or fIX optimized sequence Delta 1 (SEQ ID NO: 36), or fIX optimized sequence Delta 2 (SEQ ID NO: 37), or fIX optimized sequence Delta 3 (SEQ ID NO: 38), or fIX optimized sequence Delta 4 (SEQ ID NO: 39), or fIX optimized sequence Gamma 1 (SEQ ID NO: 40), or fIX optimized sequence Gamma 2 (SEQ ID NO: 41), or fIX optimized sequence Gamma 3 (SEQ ID NO: 42), or fIX optimized sequence Gamma 4 (SEQ ID NO: 43), or fIX optimized sequence Gamma 22, padua (SEQ ID NO: 44), or fIX optimized sequence Gamma 3 Padua (SEQ ID NO: 45), or fIX optimized sequence Gamma 87 Padua (SEQ ID NO: 46), or fIX optimized sequence Gamma 2 Padua (SEQ ID NO: 47), or fIX optimized sequence Beta 82 (SEQ ID NO: 48), of fIX optimized sequence Beta 81 Padua (SEQ ID No: 49) or fIX optimized sequence Beta 23 Padua (SEQ ID NO: 50), or fIX optimized sequence Beta 39 Padua (SEQ ID NO: 51). In some embodiments, a recombinant nucleic acid molecule is provided comprising the nucleotide sequence set forth as SEQ ID NO: 17 and/or An96 fIX (SEQ ID NO: 2), HFix-96sp3pro (SEQ ID NO: 3), or An96-hflXpro (SEQ ID NO: 4), or HFix-96appro (SEQ ID NO: 5), or hfIX-96e2pro (SEQ ID NO: 6), or hfIX-96e2V86A (SEQ ID NO: 7), or hflX-96e2V86Apro2 (SEQ ID NO: 8), or HFix-96ge2appro (SEQ ID NO: 9), or hfIX-96ge2pro (SEQ ID NO: 10), or HFix-96ge2pro2(SEQ ID NO: 11), or hfIX-96gpro (SEQ ID NO: 12), or hfIX-96pro (SEQ ID NO: 13), or HFix-96pro2 (SEQ ID NO: 14) or fIX optimized sequence Alpha 1 (SEQ ID NO: 28), or fIX optimized sequence Alpha 2 (SEQ ID NO: 29), or fIX optimized sequence Alpha 3 (SEQ ID NO: 30), or fIX optimized sequence Alpha 4 (SEQ ID NO: 31), or fIX optimized sequence Beta 1 (SEQ ID NO: 32), or fIX optimized sequence Beta 2 (SEQ ID NO: 33), or fIX optimized sequence Beta 3 (SEQ ID NO: 34), or fIX optimized sequence Beta 4 (SEQ ID NO: 35), or fIX optimized sequence Delta 1 (SEQ ID NO: 36), or fIX optimized sequence Delta 2 (SEQ ID NO: 37), or fIX optimized sequence Delta 3 (SEQ ID NO: 38), or fIX optimized sequence Delta 4 (SEQ ID NO: 39), or fIX optimized sequence Gamma 1 (SEQ ID NO: 40), or fIX optimized sequence Gamma 2 (SEQ ID NO: 41), or fIX optimized sequence Gamma 3 (SEQ ID NO: 42), or fIX optimized sequence Gamma 4 (SEQ ID NO: 43), or fIX optimized sequence Gamma 22, padua (SEQ ID NO: 44), or fIX optimized sequence Gamma 3 Padua (SEQ ID NO: 45), or fIX optimized sequence Gamma 87 Padua (SEQ ID NO: 46), or fIX optimized sequence Gamma 2 Padua (SEQ ID NO: 47), or fIX optimized sequence Beta 82 (SEQ ID NO: 48), of fIX optimized sequence Beta 81 Padua (SEQ ID No: 49) or fIX optimized sequence Beta 23 Padua (SEQ ID NO: 50), or fIX optimized sequence Beta 39 Padua (SEQ ID NO: 51). In some embodiments, CpG motifs within the codon-optimized sequences SEQ ID NOS: 3-14, 27-51, can be removed to provide a CpG deleted, liver codon optimized fIX sequence.

In some embodiments, a nucleic acid molecule is provided that encodes a protein with fIX activity comprising an amino acid sequence set forth as residues An96 fIX (SEQ ID NO: 2), SEQ ID NOS: 18-26, fIX optimized sequence Alpha (SEQ ID NO: 52), fIX optimized sequence Beta (SEQ ID NO: 53), fIX optimized sequence Delta (SEQ ID NO: 54), fIX optimized sequence Gamma (SEQ ID NO: 55), fIX optimized sequence Gamma (w/Padua) (SEQ ID NO: 56), fIX optimized sequence Beta (w/Padua) (SEQ ID NO: 57), or an amino acid sequence at least 95%, at least 96%, at least 97%, at least 98%, or at least 99% identical thereto. Residues 1-46 of SEQ ID NOS: 1, 2, 16, 18-26, and 52-57 are the fIX signal peptide and propeptide. In some embodiments, a nucleic acid molecule is provided that encodes a protein with fIX activity comprising an amino acid sequence set forth as SEQ ID NOS: 1, 2, 16, 18-26, and 52-57 (fIX variants with signal peptide and propeptide), or an amino acid sequence at least 95%, at least 96%, at least 97%, at least 98%, or at least 99% identical thereto. In alternative embodiments, a different signal peptide and/or propeptide can be used in place of the signal peptide and/or propeptide of SEQ ID NOS: 1, 2, 16, 18-26, and 52-57, such as but not limited to an IL2 signal peptide and/or factor X propeptide.

In some embodiments, CpG motifs within the codon-optimized fIX sequences, HFix-96sp3pro (SEQ ID NO: 3), or An96-hfIXpro (SEQ ID NO: 4), or hfIX-96appro (SEQ ID NO: 5), or HFix-96e2pro (SEQ ID NO: 6), or hfIX-96e2V86A (SEQ ID NO: 7), or hfIX-96e2V86Apro2 (SEQ ID NO: 8), or hfIX-96ge2appro (SEQ ID NO: 9), or hfIX-96ge2pro (SEQ ID NO: 10), or hfIX-96ge2pro2 (SEQ ID NO: 11), or hfIX-96gpro (SEQ ID NO: 12), or hfIX-96pro (SEQ ID NO: 13), or hfIX-96pro2 (SEQ ID NO: 14), or fIX optimized sequence Alpha 1 (SEQ ID NO: 28), or fIX optimized sequence Alpha 2 (SEQ ID NO: 29), or fIX optimized sequence Alpha 3 (SEQ ID NO: 30), or fIX optimized sequence Alpha 4 (SEQ ID NO: 31), or fIX optimized sequence Beta 1 (SEQ ID NO: 32), or fIX optimized sequence Beta 2 (SEQ ID NO: 33), or fIX optimized sequence Beta 3 (SEQ ID NO: 34), or fIX optimized sequence Beta 4 (SEQ ID NO: 35), or fIX optimized sequence Delta 1 (SEQ ID NO: 36), or fIX optimized sequence Delta 2 (SEQ ID NO: 37), or fIX optimized sequence Delta 3 (SEQ ID NO: 38), or fIX optimized sequence Delta 4 (SEQ ID NO: 39), or fIX optimized sequence Gamma 1 (SEQ ID NO: 40), or fIX optimized sequence Gamma 2 (SEQ ID NO: 41), or fIX optimized sequence Gamma 3 (SEQ ID NO: 42), or fIX optimized sequence Gamma 4 (SEQ ID NO: 43), or fIX optimized sequence Gamma 22, padua (SEQ ID NO: 44), or fIX optimized sequence Gamma 3 Padua (SEQ ID NO: 45), or fIX optimized sequence Gamma 87 Padua

(SEQ ID NO: 46), or fIX optimized sequence Gamma 2 Padua (SEQ ID NO: 47), or fIX optimized sequence Beta 82 (SEQ ID NO: 48), of fIX optimized sequence Beta 81 Padua (SEQ ID No: 49) or fIX optimized sequence Beta 23 Padua (SEQ ID NO: 50), or fIX optimized sequence Beta 39 Padua (SEQ ID NO: 51) can be removed to provide a CpG deleted, liver codon optimized fIX sequence.

In further embodiments, an isolated mature fIX protein is provided that is encoded by any of the fIX sequences provided herein, for example but not limited to An96 fIX (SEQ ID NO: 2), human fIX (SEQ ID NO. 42), human fIX (SEQ ID NO. 43), hfIX-96sp3pro (SEQ ID NO: 3), or An96-hfIXpro (SEQ ID NO: 4), or hfIX-96appro (SEQ ID NO: 5), or hfIX-96e2pro (SEQ ID NO: 6), or hfIX-96e2V86A (SEQ ID NO: 7), or hfIX-96e2V86Apro2 (SEQ ID NO: 8), or hfIX-96ge2appro (SEQ ID NO: 9), or hfIX-96ge2pro (SEQ ID NO: 10), or HFix-96ge2pro2 (SEQ ID NO: 11), or hfIX-96gpro (SEQ ID NO: 12), or hfIX-96pro (SEQ ID NO: 13), or hfIX-96pro2 (SEQ ID NO: 14), fIX optimized sequence Alpha (SEQ ID NO: 52), fIX optimized sequence Beta (SEQ ID NO: 53), fIX optimized sequence Delta (SEQ ID NO: 54), fIX optimized sequence Gamma (SEQ ID NO: 55), fIX optimized sequence Gamma (w/Padua) (SEQ ID NO: 56), fIX optimized sequence Beta (w/Padua) (SEQ ID NO: 57), or an amino acid sequence at least 95%, at least 96%, at least 97%, at least 98%, or at least 99% identical thereto. Residues 1-46 of SEQ ID NOS: 1, 2, 16, 18-26, and 52-57 are the fIX signal peptide and propeptide. In some embodiments, a nucleic acid molecule is provided that encodes a protein with fIX activity comprising an amino acid sequence set forth as SEQ ID NOS: 1, 2, 16, 18-26, and 52-57 (fIX variants with signal peptide and propeptide).

SEQ ID NO: 1: (With the first 46)
MQRVNMIMAESPGLITICLLGYLLSAECTVFLDHENANKI
LNRPKRYNSGKLEEFVQGNLERECMEEKCSFEEAREVFEN
TERTTEFWKQYVDGDQCESNPCLNGGSCKDDINSYECWCP
FGFEGKNCELDVTCNIKNGRCEQFCKNSADNKVVCSCTEG
YRLAENQKSCEPAVPFPCGRVSVSQTSKLTRAETVFPDVD
YVNSTEAETILDNITQSTQSFNDFTRVVGGEDAKPGQFPW
QVVLNGKVDAFCGGSIVNEKWIVTAAHCVETGVKITVVAG
EHNIEETEHTEQKRNVIRIIPHHNYNAAINKYNHDIALLE
LDEPLVLNSYVTPICIADKEYTNIFLKFGSGYVSGWGRVF
NKGRSASVLQYLRVPLVDRATCLRSTKFTIYNNMFCAGFH
EGGRDSCQGDSGGPHVTEVEGTSFLTGIISWGEECAMKGK
YGIYTKVSRYVNWIKEKTKLT
SEQ ID NO: 15: (With/Out the first 46)
YNSGKLEEFVQGNLERECMEEKCSFEEAREVFENTERTTE
FWKQYVDGDQCESNPCLNGGSCKDDINSYECWCPFGFEGK
NCELDVTCNIKNGRCEQFCKNSADNKVVCSCTEGYRLAEN
QKSCEPAVPFPCGRVSVSQTSKLTRAETVFPDVDYVNSTE
AETILDNITQSTQSFNDFTRVVGGEDAKPGQFPWQVVLNG
KVDAFCGGSIVNEKWIVTAAHCVETGVKITVVAGEHNIEE
TEHTEQKRNVIRIIPHHNYNAAINKYNHDIALLELDEPLV
LNSYVTPICIADKEYTNIFLKFGSGYVSGWGRVFNKGRSA
SVLQYLRVPLVDRATCLRSTKFTIYNNMFCAGFHEGGRDS
CQGDSGGPHVTEVEGTSFLTGIISWGEECAMKGKYGIYTK
VSRYVNWIKEKTKLT

In some embodiments, an isolated protein is provided comprising an amino acid sequence set forth as residues 47-462 of SEQ ID NO: 16 (An96 fIX Padua), and/or An96 fIX (SEQ ID NO: 2), human fIX (SEQ ID NO. 42), human fIX (SEQ ID NO. 43), hfIX-96sp3pro (SEQ ID NO: 3), or An96-hfIXpro (SEQ ID NO: 4), or hfIX-96appro (SEQ ID NO: 5), or hfIX-96e2pro (SEQ ID NO: 6), or hfIX-96e2V86A (SEQ ID NO: 7), or hfIX-96e2V86Apro2 (SEQ ID NO: 8), or hfIX-96ge2appro (SEQ ID NO: 9), or hfIX-96ge2pro (SEQ ID NO: 10), or hfIX-96ge2pro2 (SEQ ID NO: 11), or hfIX-96gpro (SEQ ID NO: 12), or hfIX-96pro (SEQ ID NO: 13), or hfIX-96pro2 (SEQ ID NO: 14), fIX optimized sequence Alpha (SEQ ID NO: 52), fIX optimized sequence Beta (SEQ ID NO: 53), fIX optimized sequence Delta (SEQ ID NO: 54), fIX optimized sequence Gamma (SEQ ID NO: 55), fIX optimized sequence Gamma (w/Padua) (SEQ ID NO: 56), fIX optimized sequence Beta (w/Padua) (SEQ ID NO: 57), or an amino acid sequence at least 95%, at least 96%, at least 97%, at least 98%, or at least 99% identical thereto and having fIX activity. In some embodiments, an isolated protein is provided comprising an amino acid sequence set forth as SEQ

ID NO: 16 (An96 fIX Padua) and/or An96 fIX (SEQ ID NO: 2), hfIX-96sp3pro (SEQ ID NO: 3), or An96-hfIXpro (SEQ ID NO: 4), or hfIX-96appro (SEQ ID NO: 5), or hfIX-96e2pro (SEQ ID NO: 6), or hfIX-96e2V86A (SEQ ID NO: 7), or hfIX-96e2V86Apro2 (SEQ ID NO: 8), or hfIX-96ge2appro (SEQ ID NO: 9), or hfIX-96ge2pro (SEQ ID NO: 10), or hfIX-96ge2pro2 (SEQ ID NO: 11), or hfIX-96gpro (SEQ ID NO: 12), or hfIX-96pro (SEQ ID NO: 13), or hfIX-96pro2 (SEQ ID NO: 14), fIX optimized sequence Alpha (SEQ ID NO: 52), fIX optimized sequence Beta (SEQ ID NO: 53), fIX optimized sequence Delta (SEQ ID NO: 54), fIX optimized sequence Gamma (SEQ ID NO: 55), fIX optimized sequence Gamma (w/Padua) (SEQ ID NO: 56), fIX optimized sequence Beta (w/Padua) (SEQ ID NO: 57), or an amino acid sequence at least 95%, at least 96%, at least 97%, at least 98%, or at least 99% identical thereto and having fIX activity.

Disclosed herein are variants of the fIX clotting factors with increased clotting factor activity relative to the corresponding native human clotting factor proteins. The variants of the fIX clotting factors have improved therapeutic properties, including improved procoagulant therapeutic properties, which compared to an unmodified fIX polypeptide, including a human fIX polypeptide. The improved properties of the disclosed fIX variants include but are not limited to increased coagulation activity, increased catalytic activity, increased resistance to heparin, and/or improved pharmacokinetic properties. The improved properties may include decreased clearance rates, enhanced recovery, and etc.

Disclosed herein are modified fIX polypeptides containing an amino acid replacement in an unmodified fIX polypeptide, wherein the amino acid replacement can be one or more of the following, wherein the dual sequence numbering is due to the fact that the fIX polypeptide contains a signal peptide and propeptide that comprises the first 46 amino acids (SEQ ID NO. 1), the first number represents the replacement with the signal peptide and the second number represents the replacement without the signal peptide (SEQ ID NO. 15): EGF2 (V132A-V86A); N313S-N267S; E323K-E277K; V326T-V280T; D338N-D292N; K339R-K293R; H361N-H315N; K362R-K316R; L367S-L321S; L366S-L320S; V368I-V322I; V367I-V321I; F399Y-F353Y; R404K-R358K; D232N-D186N; V243L-V197L; V248I-V242I; V257I-V211I; I262V-I216V; V269I-V223I; T271P-T225P; E286K-E240K; H289P-H243P; I299V-I253V; A308T-A262T; R384E-R338E or R384L-R338L.

In some embodiments, an isolated nucleic acid molecule is provided that comprises a nucleic acid sequence encoding a fIX protein comprising an amino acid sequence at least 95% identical to human fIX and further includes an amino acid replacement, wherein the amino acid replacement can be one or more of the following EGF2 (V132A-V86A); N313S-N267S; E323K-E277K; V326T-V280T; D338N-D292N; K339R-K293R; H361N-H315N; K362R-K316R; L367S-L321S; L366S-L320S; V368I-V322I; V367I-V321I; F399Y-F353Y; R404K-R358K; D232N-D186N; V243L-V197L; V248I-V242I; V257I-V211I; I262V-I216V; V269I-V223I; T271P-T225P; E286K-E240K; H289P-H243P; I299V-I253V; A308T-A262T; R384E-R338E or R384L-R338L.

In some embodiments, an isolated nucleic acid molecule is provided that comprises a nucleic acid sequence encoding a fIX protein comprising an amino acid sequence at least 95% identical to human fIX, e.g., SEQ ID No. 1 or SEQ ID NO. 15 and further includes an amino acid replacement, wherein the amino acid replacement can be one or more of the following EGF2 (V132A-V86A); N313S-N267S; E323K-E277K; V326T-V280T; D338N-D292N; K339R-K293R; H361N-H315N; K362R-K316R; L367S-L321S; L366S-L320S; V368I-V322I; V367I-V321I; F399Y-F353Y; R404K-R358K; D232N-D186N; V243L-V197L;V248I-V242I; V257I-V211I; I262V-1216V; V269I -V223I; T271P-T225P; E286K-E240K; H289P- H243P; I299V-I253V; A308T-A262T; R384E-R338E or R384L-R338L.

In some embodiments, novel fIX sequences consist of a combination of amino acid substitutions in the EGF2 (V132A-V86A) and protease domain N313S-N267S, E323K-E277K, V326T-V280T, D338N-D292N, K339R-K293R, H361N-H315N, K362R-K316R, L367S-L321S, L366S-L320S, V368I-V322I, V367I-V321I, F399Y-F353Y, R404K-R358K, D232N-D186N, V243L-V197L, V248I-V242I, V257I-V211I, I262V-I216V, V269I-V223I, T271P-T225P, E286K-E240K, H289P-H243P, I299V-I253V, A308T-A262T, R384E-R338E or R384L-R338L; or in a variation, EGF2 (V86A) and protease domain E323K-E277K, V326T-V280T, D338N-D292N, K339R-K293R, K362R-K316R, L367S-L321S (SEQ ID NO: 54), or in a variation, EGF2 (V132A-V86A) and protease domain E323K-E277K; D338N-D292N; K362R-K316R; L367S-L321S (SEQ ID NO: 55); or in a variation, EGF2 (V132A-V86A) and protease domain D338N-D292NL, 367S-L321S (SEQ ID NO: 52); or in a variation, EGF2 (V132A-V86A) and protease domain D338N-D292NL, K362R-K316R, 367S-L321S (SEQ ID NO: 53); that confer enhanced (-10-fold) fIX activity compared to human fIX by one-stage APTT-dependent coagulation assay.

The isolated proteins described above are clotting factor proteins. In several embodiments, the clotting factor protein is a mature clotting factor protein having clotting factor activity.

Thus, nucleic acid molecules (for example, cDNA or RNA molecules) encoding the disclosed novel clotting factors, as well as purified forms of the clotting factors, are provided. Nucleic acids encoding these molecules can readily be produced using the amino acid sequences provided herein and the genetic code. In several embodiments, the nucleic acid molecules can be expressed in a host cell (such as a mammalian cell) to produce a disclosed clotting factor.

The genetic code can be used to construct a variety of functionally equivalent nucleic acid sequences, such as nucleic acids which differ in sequence but which encode the same polypeptide sequence.

Nucleic acid molecules encoding the novel clotting factors disclosed herein can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements, 2017).

Nucleic acids can also be prepared by amplification methods. Amplification methods include the polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as bacteria, plant, yeast, insect and mammalian cells. DNA sequences encoding the clotting factors can be expressed in vitro by DNA transfer into a suitable host cell. The cell may be prokaryotic or eukaryotic. Numerous expression systems available for expression of proteins including E. coli, other bacterial hosts, yeast, and various higher eukaryotic cells such as the COS, CHO, HeLa and myeloma cell lines, can be used to express the disclosed novel clotting factors. Methods of stable transfer, meaning that the foreign DNA is continuously maintained in the host, are known in the art.

The expression of nucleic acids encoding the disclosed novel clotting factors described herein can be achieved by operably linking the DNA or cDNA to a promoter (which is either constitutive or inducible), followed by incorporation into an expression cassette. The promoter can be any promoter of interest, including a liver-specific promoter, such as the HCB promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, for example, a strong promoter to direct transcription, a ribosome binding site for translational initiation (e.g., internal ribosomal binding sequences), and a transcription/translation terminator. For E. coli, this can include a promoter such as the T7, trp, lac, or lambda promoters, a ribosome binding site, and preferably a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by well-known methods such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, GPt, neo, and hyg genes.

Modifications can be made to a nucleic acid encoding a polypeptide described herein without diminishing its biological activity. Some modifications can be made to facilitate the cloning, expression, or incorporation of the targeting molecule into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the disclosed novel clotting factors can be purified according to standard procedures in the art, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). The disclosed novel clotting factors need not be 100% pure. Once purified, partially or to homogeneity as desired, if to be used therapeutically, the polypeptides should be substantially free of endotoxin.

### III. Recombinant Vectors and Gene Therapy Applications

Any of the above discussed recombinant nucleic acid molecules encoding a fIX protein, or variant thereof, can be included in a vector (such as a AAV vector) for expression in a cell or a subject.

The nucleic acid sequences disclosed herein are useful in production of vectors (such as rAAV vectors), and are also useful in antisense delivery vectors, gene therapy vectors, or vaccine vectors. In certain embodiments, the disclosure provides for gene delivery vectors, and host cells which contain the nucleic acid sequences disclosed herein. In some embodiments, the selected vector may be delivered to a subject by any suitable method, including intravenous injection, ex-vivo transduction, transfection, electroporation, liposome delivery, membrane fusion techniques, high velocity DNA-coated pellets, viral infection, or protoplast fusion, to introduce a transgene into the subject.

In certain embodiments, the disclosure relates to virus particle, e.g., capsids, containing the nucleic acid sequences encoding the fIX proteins disclosed herein. The virus particles, capsids, and recombinant vectors are useful in delivery of the nucleic acid sequences encoding the fIX proteins to a target cell. The nucleic acids may be readily utilized in a variety of vector systems, capsids, and host cells. In certain embodiments, the nucleic acids are in vectors contained within a capsid comprising cap proteins, including AAV capsid proteins vp1, vp2, vp3 and hypervariable regions.

In certain embodiments, the nucleic acid sequences encoding the fIX proteins may be a part of any genetic element (vector) which may be delivered to a host cell, e.g., naked DNA, a plasmid, phage, transposon, cosmid, episome, a protein in a non-viral delivery vehicle (e.g., a lipid-based carrier), virus, etc. which transfer the sequences carried thereon.

In certain embodiments, a vector may be a lentivirus based (containing lentiviral genes or sequences) vector, e.g., having nucleic acid sequences derived from VSVG or GP64 pseudotypes or both. In certain embodiments, the nucleic acid sequences derived from VSVG or GP64 pseudotypes may be at least one or two or more genes or gene fragments of more than 1000, 500, 400, 300, 200, 100, 50, or 25 continuous nucleotides or nucleotides sequences with greater than 50, 60, 70, 80, 90, 95 or 99% identity to the gene or fragment.

In some embodiments, the nucleic acid and promotor sequences disclosed herein are useful in production of AAV vectors. AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency. The AAV genome is characterized by two inverted terminal repeats (ITRs) that flank two open reading frames (ORFs). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Daya and Berns, Clin Microbiol Rev 21(4):583-593, 2008).

The left ORF of AAV contains the Rep gene, which encodes four proteins- Rep78, Rep 68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin Microbiol Rev 21(4): 583-593, 2008).

AAV vectors typically contain a transgene expression cassette between the ITRs that replaces the rep and cap genes. Vector particles are produced by the co-transfection of cells with a plasmid containing the vector genome and a packaging/helper construct that expresses the rep and cap proteins in trans. During infection, AAV vector genomes enter the cell nucleus and can persist in multiple molecular states. One common outcome is the conversion of the AAV genome to a double-stranded circular episome by second-strand synthesis or complementary strand pairing.

In the context of AAV vectors, the disclosed vectors typically have a recombinant genome comprising the following structure:

(5′ AAV ITR)-(promoter)-(transgene)-(3′ AAV ITR)

As discussed above, these recombinant AAV vectors contain a transgene expression cassette between the ITRs that replaces the rep and cap genes. Vector particles are produced, for example, by the co-transfection of cells with a plasmid containing the recombinant vector genome and a packaging/helper construct that expresses the rep and cap proteins in trans.

The transgene can be flanked by regulatory sequences such as a 5′ Kozak sequence and/or a 3′ polyadenylation signal.

The AAV ITRs, and other selected AAV components described herein, may be readily selected from among any AAV serotype, including, without limitation, AAV1, AAV2, AAV3, AAV4, AAV5, AAV6, AAV7, AAV8, AAV9 and function variants thereof. These ITRs or other AAV components may be readily isolated using techniques available to those of skill in the art from an AAV serotype. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

In some embodiments, the recombinant AAV vector genome can have a liver-specific promoter, such as any one of the HCB, HSh-HCB, 5′HSh-HCB, 3′HSh-HCB, ABP-HP1-God-TSS, HSh-SynO-TSS, or sHS-SynO-TSS promoters set forth in WO 2016/168728, which is incorporated by reference herein in its entirety.

AAV is currently one of the most frequently used viruses for gene therapy. Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. Because of the advantageous features of AAV, the present disclosure contemplates the use of AAV for the recombinant nucleic acid molecules and methods disclosed herein.

AAV possesses several desirable features for a gene therapy vector, including the ability to bind and enter target cells, enter the nucleus, the ability to be expressed in the nucleus for a prolonged period of time, and low toxicity. However, the small size of the AAV genome limits the size of heterologous DNA that can be incorporated. To minimize this problem, AAV vectors have been constructed that do not encode Rep and the integration efficiency element (IEE). The ITRs are retained as they are cis signals required for packaging (Daya and Berns, Clin Microbiol Rev 21(4):583-593, 2008).

Methods for producing rAAV suitable for gene therapy are known (see, for example, U.S. patent application Ser. Nos. 2012/0100606; 2012/0135515; 2011/0229971; and 2013/0072548; and Ghosh et al., Gene Ther 13(4):321-329, 2006), and can be utilized with the recombinant nucleic acid molecules and methods disclosed herein.

In some embodiments, the nucleic acids disclosed herein are part of an expression cassette or transgene. See e.g., US Pat. App. Pub. 20150139953. The expression cassette is composed of a transgene and regulatory sequences, e.g., promotor and 5′ and 3′ AAV inverted terminal repeats (ITRs). In one desirable embodiment, the ITRs of AAV serotype 2 or 8 are used. However, ITRs from other suitable serotypes may be selected. An expression cassette is typically packaged into a capsid protein and delivered to a selected host cell.

In some embodiments, the disclosure provides for a method of generating a recombinant adeno-associated virus (AAV) having an AAV serotype capsid, or a portion thereof. Such a method involves culturing a host cell which contains a nucleic acid sequence encoding an adeno-associated virus (AAV) serotype capsid protein; a functional rep gene; an expression cassette composed of AAV inverted terminal repeats (ITRs) and a transgene; and sufficient helper functions to permit packaging of the expression cassette into the AAV capsid protein. See e.g., US Pat. App. Pub. 20150139953.

The components for culturing in the host cell to package an AAV expression cassette in an AAV capsid may be provided to the host cell in trans. Alternatively, any one or more of the components (e.g., expression cassette, rep sequences, cap sequences, and/or helper functions) may be provided by a stable host cell which has been engineered to contain one or more of the required components using methods known to those of skill in the art. Most suitably, such a stable host cell will contain the component(s) under the control of an inducible promoter. However, the required component(s) may be under the control of a constitutive promoter. In still another alternative, a selected stable host cell may contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contains the rep and/or cap proteins under the control of inducible promoters. Still other stable host cells may be generated by one of skill in the art.

In some embodiments, the disclosure relates to recombinant vectors comprising a liver specific promotor nucleic acid sequence in operable combination with transgene. The transgene is a nucleic acid sequence, heterologous to the vector sequences flanking the transgene, which encodes a novel fIX protein as disclosed herein, and optionally one or more additional proteins of interest. The nucleic acid coding sequence is operatively linked to regulatory components in a manner which permits transgene transcription, translation, and/or expression in a host cell.

The expression cassette can be carried on any suitable vector, e.g., a plasmid, which is delivered to a host cell. The plasmids useful in this disclosure may be engineered such that they are suitable for replication and, optionally, integration in prokaryotic cells, mammalian cells, or both. These plasmids (or other vectors carrying the 5′ AAV ITR-heterologous molecule-3′ ITR) contain sequences permitting replication of the expression cassette in eukaryotes and/or prokaryotes and selection markers for these systems. Preferably, the molecule carrying the expression cassette is transfected into the cell, where it may exist transiently. Alternatively, the expression cassette (carrying the 5′ AAV ITR-heterologous molecule-3′ ITR) may be stably integrated into the genome of the host cell, either chromosomally or as an episome. In certain embodiments, the expression cassette may be present in multiple copies, optionally in head-to-head, head-to-tail, or tail-to-tail concatamers. Suitable transfection techniques are known and may readily be utilized to deliver the expression cassette to the host cell.

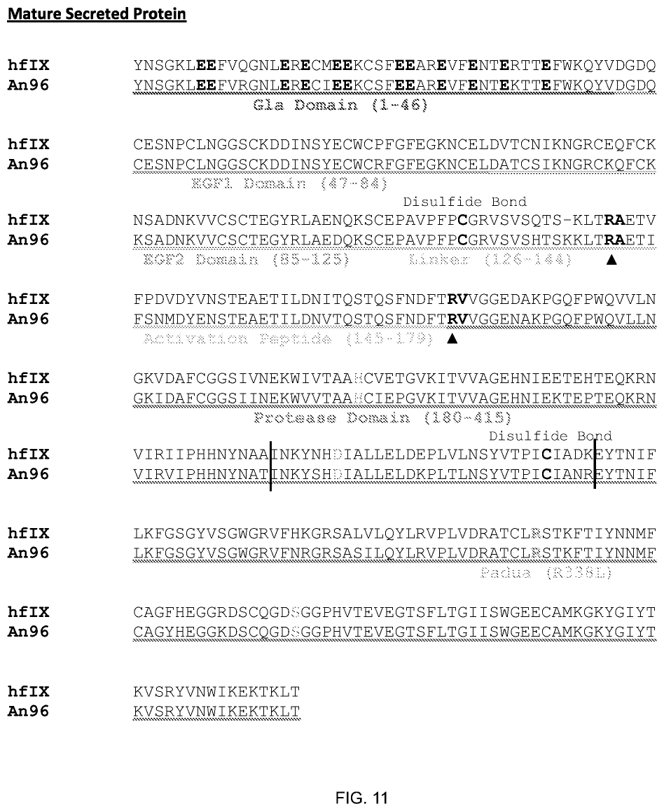

In some embodiments, substitutions are based on ancestral fIX sequences. Ancestral fIX sequences were identified through ASR and synthesized de novo for in vitro expression studies. Ancestral sequence 96 (An96) was identified to have greater activity than human fIX and comparable activity to fIX-Padua (R384L-R338L). An96 is 90% human at the amino acid level and through domain swapping studies between human flX and An96, domains that confer greater activity were identified to be the EGF2 and protease domains of An96. Briefly, constructs were generated by domain swapping and cloned into AAV2 expression plasmids. Huh-7 liver cells were transiently transfected with each construct, and expression of flX measured from conditioned medium by one-stage APTT-dependent clotting assay. Activity was normalized to hfIX expression levels and compared to An96 expression levels. In a series of reiterative experiments, the EGF2 and protease domains of An96 were identified to confer enhanced levels of fIX expression when substituted into hfIX. See FIG. 12 for construct maps showing various domain swaps. Through amino acid substitution studies, the V132A-V86A single point mutation in the EGF2 domain and the N313S-N267S; E323K-E277K; V326T-V280T; D338N-D292N; K339R-K293R; H361N-H315N; K362R-K316R; L367S-L321S; L366S-L320S; V368I-V322I; V367I-V321I; F399Y-F353Y; R404K-R358K; D232N-D186N; V243L-V197L;V248I-V242I; V257I-V211I; I262V-1216V; V269I-V223I; T271P-T225P; E286K-E240K; H289P-H243P; I299V-I253V ; A308T-A262T; R384E-R338E or R384L-R338L mutations in the protease domain confer ˜10-fold fIX activity compared to human fIX equivalent to that of An96. Upon more single point mutations studies, amino acid replacements EGF2 (V132A-V86A); E323K-E277K; V326T-V280T; D338N-D292N; K339R-K293R; K362R-K316R; and L367S-L321S (See, e.g., SEQ ID NO: 54, fIX Delta) were found to confer ˜10-fold fIX activity compared to human fIX equivalent to or surpassing that of An96. Upon more single point mutations studies, amino acid replacements EGF2 (V132A-V86A); D338N- D292N; K362R-K316R; and L367S-L321S (See, for example, SEQ ID No. 53, fIX Beta) were found to confer ˜10-fold fIX activity compared to human fIX equivalent to or surpassing that of An96. Upon more single point mutations studies, amino acid replacements D338N-D292N and L367S-L321S (See, for example, SEQ ID No. 52, fIX Alpha) were found to confer ˜10-fold fIX activity compared to human fIX equivalent to or surpassing that of An96. Upon more single point mutations studies, amino acid replacements EGF2 (V132A-V86A); E323K-E277K; D338N-D292N; K362R-K316R; and L367S-L321S (See, for example, SEQ ID No. 55, fIX Gamma) were found to confer ˜10-fold fIX activity compared to human fIX equivalent to or surpassing that of An96.

Upon further amino acid replacements, EGF2 (V132A-V86A); E323K-E277K; D338N- D292N; K362R-K316R; L367S-L321S; and V132A-V86A (See, for example, SEQ ID No. 56, fIX Gamma (with)) were found to confer ˜10-fold fIX activity compared to human fIX equivalent to or surpassing that of An96.

Upon further amino acid replacements, EGF2 (V132A-V86A); D338N-D292N; K362R-K316R; L367S-L321S; and V132A-V86A (See, for example, SEQ ID No. 57, fIX Beta (with)) were found to confer ˜10-fold fIX activity compared to human fIX equivalent to or surpassing that of An96. The fIX tmnsgene(s) are cloned into AAV-expression plasmid containing desired ITR and AAV is manufactured for liver-directed in vivo delivery.

Generally, when delivering the vector comprising the expression cassette by transfection, the vector and the relative amounts of vector DNA to host cells may be adjusted, taking into consideration such factors as the selected vector, the delivery method and the host cells selected. In addition to the expression cassette, the host cell contains the sequences which drive expression of the AAV capsid protein in the host cell and rep sequences of the same serotype as the serotype of the AAV ITRs found in the expression cassette, or a cross-complementing serotype. Although the molecule(s) providing rep and cap may exist in the host cell transiently (i.e., through transfection), it is preferred that one or both of the rep and cap proteins and the promoter(s) controlling their expression be stably expressed in the host cell, e.g., as an episome or by integration into the chromosome of the host cell.

The packaging host cell also typically contains helper functions in order to package the rAAV of the disclosure. Optionally, these functions may be supplied by a herpesvirus. Most desirably, the necessary helper functions are each provided from a human or non-human primate adenovirus source, such as those described above and/or are available from a variety of sources, including the American Type Culture Collection (ATCC), Manassas, Va. (US). The desired helper functions, can be provided using any means that allows their expression in a cell.

Introduction into the host cell of the vector may be achieved by any means known in the art or as disclosed above, including transfection, infection, electroporation, liposome delivery, membrane fusion techniques, high velocity DNA-coated pellets, viral infection and protoplast fusion, among others. One or more of the adenoviral genes may be stably integrated into the genome of the host cell, stably expressed as episomes, or expressed transiently. The gene products may all be expressed transiently, on an episome or stably integrated, or some of the gene products may be expressed stably while others are expressed transiently. Furthermore, the promoters for each of the adenoviral genes may be selected independently from a constitutive promoter, an inducible promoter or a native adenoviral promoter. The promoters may be regulated by a specific physiological state of the organism or cell (i.e., by the differentiation state or in replicating or quiescent cells) or by exogenously added factors, for example.

The AAV techniques can be adapted for use in these and other viral vector systems for in vitro, ex vivo or in vivo gene delivery. The in certain embodiments the disclosure contemplates the use of nucleic acids and vectors disclosed herein in a variety of rAAV and non-rAAV vector systems. Such vectors systems may include, e.g., lentiviruses, retroviruses, poxviruses, vaccinia viruses, and adenoviral systems, among others.

In some embodiments, it is contemplated that viral particles, nucleic acids and vectors disclosed herein are useful for a variety of purposes, including for delivery of therapeutic molecules for gene expression of therapeutic proteins.

Therapeutic proteins encoded by the nucleic acids (e.g., operably in combination with promoters) reported herein include those used for treatment of clotting disorders, including hemophilia B (e.g., using a fIX protein as provided herein), hemophilia A (e.g., using a fVIII protein as provided herein), and congenital proconvertin deficiency (e.g., using a fVII protein as provided herein)

In some embodiments, a method of inducing blood clotting in a subject in need thereof is provided. The method comprises administering to the subject a therapeutically effective amount of a vector (such as an AAV vector, a lentiviral vector, or a retroviral vector) encoding a nucleic acid sequences encoding the fIX proteins as described herein. In some embodiments, the subject is a subject with a clotting disorder, such as hemophilia A or hemophilia B. In some embodiments, the clotting disorder is hemophilia B and the subject is administered a vector comprising a nucleic acid molecule encoding a protein with fIX activity.

A treatment option for a patient diagnosed with hemophilia B is the exogenous administration of recombinant fIX sometimes referred to as fIX replacement therapy. In some embodiments, a patient with hemophilia A or hemophilia B can be treated by administration of a recombinant fVIII or fIX protein as described herein. In some patients, these therapies can lead to the development of antibodies that bind to the administered clotting factor. Subsequently, the clotting factor-antibody bound conjugates, typically referred to as inhibitors, interfere with or retard the ability of the exogenous clotting factor to cause blood clotting Inhibitory autoantibodies also sometimes occur spontaneously in a subject that is not genetically at risk of having a clotting disorder such as hemophilia, termed acquired hemophilia Inhibitory antibodies assays are typically performed prior to exogenous clotting factor treatment in order to determine whether the anti-coagulant therapy will be effective.

A “Bethesda assay” has historically been used to quantitate the inhibitory strength the concentration of fVIII binding antibodies. In the assay, serial dilutions of plasma from a patient, e.g., prior to having surgery, are prepared and each dilution is mixed with an equal volume of normal plasma as a source of fVIII. After incubating for a couple hours, the activities of fVIII in each of the diluted mixtures are measured. Having antibody inhibitor concentrations that prevent fVIII clotting activity after multiple repeated dilutions indicates a heightened risk of uncontrolled bleeding. Patients with inhibitor titers after about ten dilutions are felt to be unlikely to respond to exogenous fVIII infusions to stop bleeding. A Bethesda titer is defined as the reciprocal of the dilution that results in 50% inhibition of FVIII activity present in normal human plasma. A Bethesda titer greater than 10 is considered the threshold of response to FVIII replacement therapy.

In certain embodiments, the disclosure relates to methods of inducing blood clotting comprising administering an effective amount of a viral particle or capsid comprising a vector comprising a nucleic acid encoding a blood clotting factor as disclosed herein to a subject in need thereof.

In certain embodiments, the subject is diagnosed with hemophilia A or B or acquired hemophilia or unlikely to respond to exogenous clotting factor infusions (e.g., based on a Bethesda assay result).

In some embodiments, this disclosure relates to methods of gene transfer for the treatment of hemophilia B using an adeno-associated viral (AAV) vector encoding human fIX as the gene delivery vehicle. While several such AAV-based gene therapies for hemophilia B have entered into human clinical trials, they have been hampered by low expression of the therapeutic protein, clotting fIX, after administration of the virus resulting on only partial correction of the disease. AAV vector toxicity limits the dose of the virus that may be safely administered. Typically, the vector provides efficacious expression of fIX at viral doses below the threshold of toxicity.

In some embodiments, this disclosure relates to methods of gene transfer for the treatment of hemophilia B using a lentiviral vector encoding human fIX as the gene delivery vehicle. Delivery of the lentiviral vector encoding the transgene can be, for example, by direct administration to the subject, or by ex vivo transduction and transplantation of hematopoietic stem and progenitor cells with the vector. Typically, the vector provides efficacious expression of fIX at viral doses below the threshold of toxicity.

In some embodiments, recombinant virus particles, capsids, or vectors comprising nucleic acids disclosed herein can be delivered to liver via the hepatic artery, the portal vein, or intravenously to yield therapeutic levels of therapeutic proteins or clotting factors in the blood. The capsid or vector is preferably suspended in a physiologically compatible carrier, may be administered to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, sesame oil, and water.

Optionally, the compositions of the disclosure may contain other pharmaceutically acceptable excipients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin

The recombinant virus particles, capsids, or vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the liver (optionally via the hepatic artery) or lung), oral, inhalation, intranasal, intratracheal, intraarterial, intraocular, intravenous, intramuscular, subcutaneous, intradermal, and other parental routes of administration. Routes of administration may be combined, if desired.

Dosages of the recombinant virus particles, capsids, or vectors will depend primarily on factors such as the condition being treated, the age, weight and health of the patient, and may thus vary among patients. For example, a therapeutically effective human dosage of the viral vector is generally in the range of from about 0.1 ml to about 100 ml of solution containing concentrations of from about 1×10⁹ to 1×10¹⁶ genomes virus vector.

Recombinant viral vectors of the disclosure provide an efficient gene transfer vehicle which can deliver a selected protein to a selected host cell in vivo or ex vivo even where the organism has neutralizing antibodies to the protein. In one embodiment, the vectors disclosed herein and the cells are mixed ex vivo; the infected cells are cultured using conventional methodologies; and the transduced cells are re-infused into the patient.

### EXAMPLES

The following examples are provided to illustrate certain particular features and/or embodiments. These examples should not be construed to limit the disclosure to the particular features or embodiments described.

### EXAMPLE 1

### Bioengineering Coagulation Factor IX through Ancestral Protein Reconstruction

This example illustrates the optimization of fIX sequences to improve clotting factor activity, utility for protein expression and therapeutic applications such as gene therapy.

The development of transformative hemophilia therapeutics has been hindered by the size, instability, immunogenicity and biosynthetic inefficiency of coagulation factors such as fIX for treatment of hemophilia B. Factor IX is a very large glycoprotein, it is highly sensitive to disruptive mutations and there are no high-resolution structures of the X-ase complex available. These limitations challenge fIX protein engineering. Accordingly, it is desirable to find additional fIX sequences that have increased activity (for example, due to increased serum half-life or increased enzymatic activity) because it is possible that the frequency of infusion may be lessened while still achieving full prophylaxis.

To search for additional fIX sequences that may facilitate improved clotting factor replacement therapy for hemophilia B, a mammalian fIX phylogenetic tree with corresponding ancestral node (An) sequences was constructed through Bayesian inference using both DNA and amino acid-based models in PAML Version 4.1. Initially, nine An-fIX sequences were selected for reconstruction, as follows:

An63 fIX
(SEQ ID NO: 19)
MQCLNMIMAESPGLITICLLGYLLSAECTVFLDHENATKILNRPKRYNSGKLEEFVQGNLERECIEEKCSF
EEAREVFENTEKTTEFWKQYVDGDQCESNPCLNGGMCKDDINSYECWCQVGFEGKNCELDATCSIKNGRCKQFCKKGA
DNKVVCSCTTGYRLAEDQKSCEPAVPFPCGRVSVSHTSKKLTRAETIFSNMDYENSTEAETILDNVTQSTQSFNDFTR
VVGGENAKPGQFPWQVLLNGKIDAFCGGSIINEKWVVTAAHCIEPGVKITVVAGEHNTEETEHTEQKRNVIRVIPHHS
YNATINKYSHDIALLELDKPLTLNSYVTPICIANREYTNIFLKFGSGYVSGWGKVFNRGRSASILQYLKVPLVDRATC
LRSTKFTIYNNMFCAGYHEGGKDSCQGDSGGPHVTEVEGTSFLTGIISWGEECAMKGKYGIYTKVSRYVNWIKEKTKL
T
An65 fIX
(SEQ ID NO: 20)
MQCLNMIMAESPGLITICLLGYLLSAECTVFLDHENATKILNRPKRYNSGKLEEFVQGNLERECIEEKCSF
EEAREVFENTEKTTEFWKQYVDGDQCESNPCLNGGLCKDDINSYECWCQVGFEGKNCELDATCSIKNGRCKQFCKKGA
DNKVVCSCTTGYRLAEDQKSCEPAVPFPCGRVSVSHTSTKLTRAETIFSNMDYENSTEAETILDNVTQSTQSFNDFTR
VVGGENAKPGQFPWQVLLNGKIDAFCGGSIINEKWVVTAAHCIEPGVKITVVAGEHNTEETEHTEQKRNVIRVIPHHS
YNATINKYSHDIALLELDKPLTLNSYVTPICIADREYTNIFLKFGSGYVSGWGKVFNRGRSASILQYLKVPLVDRATC
LRSTKFTIYNNMFCAGFHEGGKDSCQGDSGGPHVTEVEGTSFLTGIISWGEECAMKGKYGIYTKVSRYVNWIKEKTKL
T
An70 fIX
(SEQ ID NO: 21)
MQCLNMIMAESPGLVTICLLGYLLSAECTVFLDRENATKILNRPKRYNSGKLEEFVRGNLERECIEEKCSF
EEAREVFENTEKTTEFWKQYVDGDQCESNPCLNGGLCKDDINSYECWCQVGFEGKNCELDATCSIKNGRCKQFCKKGA
DNKVVCSCTTGYRLAEDQKSCEPAVPFPCGRVSVSHTSTKLTRAETIFSNMDYENSTEAEIILDNVTQSNQSFNDFTR
IVGGENAKPGQFPWQVLLNGKIDAFCGGSIINEKWVVTAAHCIEPGVKITVVAGEHNTEETEPTEQKRNVIRAIPHHS
YNATVNKYSHDIALLELDEPLTLNSYVTPICIADREYTNIFLKFGSGYVSGWGKVFNRGRSASILQYLKVPLVDRATC
LRSTKFTIYNNMFCAGFHEGGKDSCQGDSGGPHVTEVEGTSFLTGIISWGEECAVKGKYGIYTKVSRYVNWIKEKTKL
T
An84 fIX
(SEQ ID NO: 22)
MQCLNMIMAESPGLITICLLGYLLSAECTVFLDHENATKILNRPKRYNSGKLEEFVQGNLERECIEEKCSF
EEAREVFENTEKTTEFWKQYVDGDQCESNPCLNGGSCKDDINSYECWCQVGFEGKNCELDATCSIKNGRCKQFCKKGA
DNKVVCSCTEGYRLAEDQKSCEPAVPFPCGRVSVSHTSKKLTRAETIFSNMDYENSTEAETILDNVTQSTQSFNDFTR
VVGGENAKPGQFPWQVLLNGKIDAFCGGSIINEKWVVTAAHCIEPGVKITVVAGEHNIEETEHTEQKRNVIRVIPHHN
YNATINKYSHDIALLELDKPLTLNSYVTPICIANREYTNIFLKFGSGYVSGWGRVFNRGRSASILQYLRVPLVDRATC
LRSTKFTIYNNMFCAGYHEGGKDSCQGDSGGPHVTEVEGTSFLTGIISWGEECAMKGKYGIYTKVSRYVNWIKEKTKL
T
An88 fIX
(SEQ ID NO: 23)
MQHLNTIMAESPGLITIFLLGYLLSAECAVFLDRENATKILTRPKRYNSGKLEEFVQGNLERECIEERCSF
EEAREVFENTEKTTEFWKQYVDGDQCESNPCLNGGKCKDDINSYECWCQVGFEGRNCELDATCNIKNGRCKQFCKNGA
DNKVICSCTEGYQLAEDQKSCEPAVPFPCGRVSVSYSSKKLTRAETIFSNMDYENSTEAETILDNVTENSESLNDFTR
VVGGENAKPGQIPWQVILNGEIEAFCGGAIINEKWVVTAAHCLKPGDKIEVVAGEYNIDEKEDTEQRRNVIRTIPHHH
YNATINKYSHDIALLELDKPLILNSYVTPICVANREYTNIFLKFGSGYVSGWGKVFNKGRQASILQYLRVPLVDRATC
LRSTTFTIYNNMFCAGYREGGKDSCEGDSGGPHVTEVEGTSFLTGIISWGEECAMKGKYGIYTKVSRYVNWIKEKTKL
T
An95 fIX
(SEQ ID NO: 24)
MQCLNMIMAESPGLITICLLGYLLSAECTVFLDHENATKILNRPKRYNSGKLEEFVQGNLERECIEEKCSF
EEAREVFENTEKTTEFWKQYVDGDQCESNPCLNGGSCKDDINSYECWCQVGFEGKNCELDATCSIKNGRCKQFCKKGA
DNKVVCSCTEGYRLAEDQKSCEPAVPFPCGRVSVSHTSKKLTRAETIFSNMDYENSTEAETILDNVTQSTQSFNDFTR
VVGGENAKPGQFPWQVLLNGKIDAFCGGSIINEKWVVTAAHCIEPGVKITVVAGEHNIEETEHTEQKRNVIRVIPHHN
YNATINKYSHDIALLELDKPLTLNSYVTPICIANREYTNIFLKFGSGYVSGWGRVFNRGRSASILQYLRVPLVDRATC
LRSTKFTIYNNMFCAGYHEGGKDSCQGDSGGPHVTEVEGTSFLTGIISWGEECAMKGKYGIYTKVSRYVNWIKEKTKL
T
An96 fIX
(SEQ ID NO: 2)
MQCLNMIMAESPGLITICLLGYLLSAECTVFLDHENATKILNRPKRYNSGKLEEFVRGNLERECIEEKCSF
EEAREVFENTEKTTEFWKQYVDGDQCESNPCLNGGSCKDDINSYECWCRFGFEGKNCELDATCSIKNGRCKQFCKKSA
DNKVVCSCTEGYRLAEDQKSCEPAVPFPCGRVSVSHTSKKLTRAETIFSNMDYENSTEAETILDNVTQSTQSFNDFTR
VVGGENAKPGQFPWQVLLNGKIDAFCGGSIINEKWVVTAAHCIEPGVKITVVAGEHNIEKTEPTEQKRNVIRVIPHHN
YNATINKYSHDIALLELDKPLTLNSYVTPICIANREYTNIFLKFGSGYVSGWGRVFNRGRSASILQYLRVPLVDRATC
LRSTKFTIYNNMFCAGYHEGGKDSCQGDSGGPHVTEVEGTSFLTGIISWGEECAMKGKYGIYTKVSRYVNWIKEKTKL
T
An97 fIX
(SEQ ID NO: 25)
MQCLNMIMAESPGLITICLLGYLLSAECTVFLDHENANKILNRPKRYNSGKLEEFVRGNLERECIEEKCSF
EEAREVFENTEKTTEFWKQYVDGDQCESNPCLNGGSCKDDINSYECWCRFGFEGKNCELDATCSIKNGRCKQFCKKSA
DNKVVCSCTEGYRLAEDQKSCEPAVPFPCGRVSVSHTSKLTRAETIFSNMDYENSTEAETILDNVTQSTQSFNDFTRV
VGGENAKPGQFPWQVLLNGKIDAFCGGSIINEKWVVTAAHCIEPGVKITVVAGEHNIEKTEPTEQKRNVIRVIPHHNY
NATINKYSHDIALLELDKPLTLNSYVTPICIADREYTNIFLKFGSGYVSGWGRVFNRGRSASILQYLRVPLVDRATCL
RSTKFTIYNNMFCAGYHEGGKDSCQGDSGGPHVTEVEGTSFLTGIISWGEECAMKGKYGIYTKVSRYVNWIKEKTKLT
An102 fIX
(SEQ ID NO: 26)
MQRVNMIMAESPGLITICLLGYLLSAECTVFLDHENANKILNRPKRYNSGKLEEFVQGNLERECMEEKCSF
EEAREVFENTEKTTEFWKQYVDGDQCESNPCLNGGSCKDDINSYECWCPFGFEGKNCELDVTCNIKNGRCKQFCKNSA
DNKVVCSCTEGYRLAENQKSCEPAVPFPCGRVSVSQTSKLTRAETVFPDVDYVNSTEAETILDNITQSTQSFNDFTRV
VGGEDAKPGQFPWQVVLNGKVDAFCGGSIVNEKWVVTAAHCIETGVKITVVAGEHNIEETEHTEQKRNVIRIIPHHNY
NATINKYNHDIALLELDEPLVLNSYVTPICIADKEYTNIFLKFGSGYVSGWGRVFNKGRSASVLQYLRVPLVDRATCL
RSTKFTIYNNMFCAGFHEGGRDSCQGDSGGPHVTEVEGTSFLTGIISWGEECAMKGKYGIYTKVSRYVNWIKEKTKLT

To search for additional fIX sequences that may facilitate improved clotting factor replacement therapy for hemophilia B, a mammalian fIX phylogenetic tree with corresponding ancestral node (An) sequences was constructed through Bayesian inference using both DNA and amino acid-based models in a custom software program developed by the inventors. Seven further An-fIX sequences were selected for reconstruction, as follows:

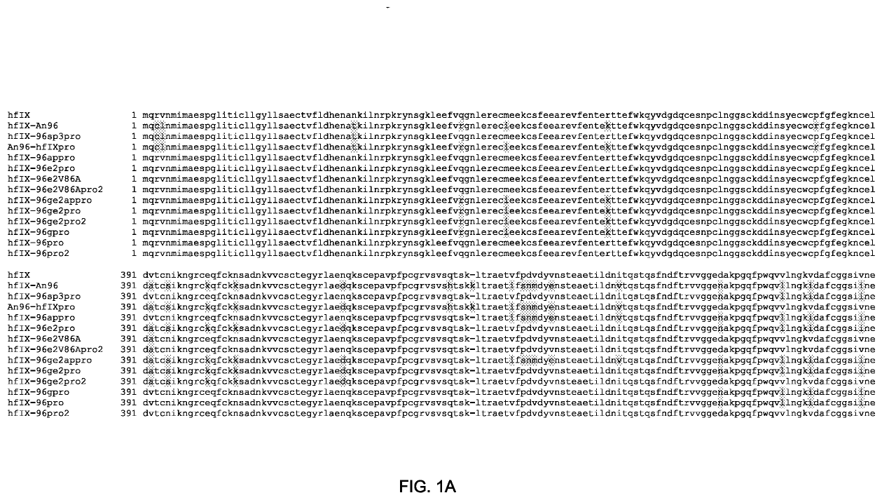

fIX Alpha (no Padua)
(SEQ ID NO: 52)
MQRVNMIMAESPGLITICLLGYLLSAECTVFLDHENANKI
LNRPKRYNSGKLEEFVQGNLERECMEEKCSFEEAREVFEN
TERTTEFWKQYVDGDQCESNPCLNGGSCKDDINSYECWCP
FGFEGKNCELDATCNIKNGRCEQFCKNSADNKVVCSCTEG
YRLAENQKSCEPAVPFPCGRVSVSQTSKLTRAETVFPDVD
YVNSTEAETILDNITQSTQSFNDFTRVVGGEDAKPGQFPW
QVVLNGKVDAFCGGSIVNEKWIVTAAHCVETGVKITVVAG
EHNIEETEHTEQKRNVIRIIPHHNYNAAINKYNHDIALLE
LDEPLVLNSYVTPICIANKEYTNIFLKFGSGYVSGWGRVF
HKGRSASVLQYLRVPLVDRATCLRSTKFTIYNNMFCAGFH
EGGRDSCQGDSGGPHVTEVEGTSFLTGIISWGEECAMKGK
YGIYTKVSRYVNWIKEKTKLT*
fIX Beta (no Padua)
(SEQ ID NO: 53)
MQRVNMIMAESPGLITICLLGYLLSAECTVFLDHENANKI
LNRPKRYNSGKLEEFVQGNLERECMEEKCSFEEAREVFEN
TERTTEFWKQYVDGDQCESNPCLNGGSCKDDINSYECWCP
FGFEGKNCELDATCNIKNGRCEQFCKNSADNKVVCSCTEG
YRLAENQKSCEPAVPFPCGRVSVSQTSKLTRAETVFPDVD
YVNSTEAETILDNITQSTQSFNDFTRVVGGEDAKPGQFPW
QVVLNGKVDAFCGGSIVNEKWIVTAAHCVETGVKITVVAG
EHNIEETEHTEQKRNVIRIIPHHNYNAAINKYNHDIALLE
LDEPLVLNSYVTPICIANKEYTNIFLKFGSGYVSGWGRVF
HRGRSASVLQYLRVPLVDRATCLRSTKFTIYNNMFCAGFH
EGGRDSCQGDSGGPHVTEVEGTSFLTGIISWGEECAMKGK
YGIYTKVSRYVNWIKEKTKLT*
fIX Delta (no Padua)
(SEQ ID NO: 54)
MQRVNMIMAESPGLITICLLGYLLSAECTVFLDHENANKI
LNRPKRYNSGKLEEFVQGNLERECMEEKCSFEEAREVFEN
TERTTEFWKQYVDGDQCESNPCLNGGSCKDDINSYECWCP
FGFEGKNCELDATCNIKNGRCEQFCKNSADNKVVCSCTEG
YRLAENQKSCEPAVPFPCGRVSVSQTSKLTRAETVFPDVD
YVNSTEAETILDNITQSTQSFNDFTRVVGGEDAKPGQFPW
QVVLNGKVDAFCGGSIVNEKWIVTAAHCVETGVKITVVAG
EHNIEETEHTEQKRNVIRIIPHHNYNAAINKYNHDIALLE
LDKPLTLNSYVTPICIANREYTNIFLKFGSGYVSGWGRVF
HRGRSASVLQYLRVPLVDRATCLRSTKFTIYNNMFCAGFH
EGGRDSCQGDSGGPHVTEVEGTSFLTGIISWGEECAMKGK
YGIYTKVSRYVNWIKEKTKLT*
fIX Gamma (no Padua)
(SEQ ID NO: 55)
MQRVNMIMAESPGLITICLLGYLLSAECTVFLDHENANKI
LNRPKRYNSGKLEEFVQGNLERECMEEKCSFEEAREVFEN
TERTTEFWKQYVDGDQCESNPCLNGGSCKDDINSYECWCP
FGFEGKNCELDATCNIKNGRCEQFCKNSADNKVVCSCTEG
YRLAENQKSCEPAVPFPCGRVSVSQTSKLTRAETVFPDVD
YVNSTEAETILDNITQSTQSFNDFTRVVGGEDAKPGQFPW
QVVLNGKVDAFCGGSIVNEKWIVTAAHCVETGVKITVVAG
EHNIEETEHTEQKRNVIRIIPHHNYNAAINKYNHDIALLE
LDKPLVLNSYVTPICIANKEYTNIFLKFGSGYVSGWGRVF
HRGRSASVLQYLRVPLVDRATCLRSTKFTIYNNMFCAGFH
EGGRDSCQGDSGGPHVTEVEGTSFLTGIISWGEECAMKGK
YGIYTKVSRYVNWIKEKTKLT*
fIX Gamma (with Padua)
(SEQ ID NO: 56)
MQRVNMIMAESPGLITICLLGYLLSAECTVFLDHENANKI
LNRPKRYNSGKLEEFVQGNLERECMEEKCSFEEAREVFEN
TERTTEFWKQYVDGDQCESNPCLNGGSCKDDINSYECWCP
FGFEGKNCELDATCNIKNGRCEQFCKNSADNKVVCSCTEG
YRLAENQKSCEPAVPFPCGRVSVSQTSKLTRAETVFPDVD
YVNSTEAETILDNITQSTQSFNDFTRVVGGEDAKPGQFPW
QVVLNGKVDAFCGGSIVNEKWIVTAAHCVETGVKITVVAG
EHNIEETEHTEQKRNVIRIIPHHNYNAAINKYNHDIALLE
LDKPLVLNSYVTPICIANKEYTNIFLKFGSGYVSGWGRVF
HRGRSASVLQYLRVPLVDRATCLLSTKFTIYNNMFCAGFH
EGGRDSCQGDSGGPHVTEVEGTSFLTGIISWGEECAMKGK
YGIYTKVSRYVNWIKEKTKLT*
fIX Beta (with Padua)
(SEQ ID NO: 57)
MQRVNMIMAESPGLITICLLGYLLSAECTVFLDHENANKI
LNRPKRYNSGKLEEFVQGNLERECMEEKCSFEEAREVFEN
TERTTEFWKQYVDGDQCESNPCLNGGSCKDDINSYECWCP
FGFEGKNCELDATCNIKNGRCEQFCKNSADNKVVCSCTEG
YRLAENQKSCEPAVPFPCGRVSVSQTSKLTRAETVFPDVD
YVNSTEAETILDNITQSTQSFNDFTRVVGGEDAKPGQFPW
QVVLNGKVDAFCGGSIVNEKWIVTAAHCVETGVKITVVAG
EHNIEETEHTEQKRNVIRIIPHHNYNAAINKYNHDIALLE
LDEPLVLNSYVTPICIANKEYTNIFLKFGSGYVSGWGRVF
HRGRSASVLQYLRVPLVDRATCLLSTKFTIYNNMFCAGFH
EGGRDSCQGDSGGPHVTEVEGTSFLTGIISWGEECAMKGK
YGIYTKVSRYVNWIKEKTKLT*
FIGS. 1A and 1B shows a sequence alignment
of the above fIX proteins with hfIX sequence,
which is provided as SEQ ID NO: 1:
MQRVNMIMAESPGLITICLLGYLLSAECTVFLDHENANKI
LNRPKRYNSGKLEEFVQGNLERECMEEKCSFEEAREVFEN
TERTTEFWKQYVDGDQCESNPCLNGGSCKDDINSYECWCP
FGFEGKNCELDVTCNIKNGRCEQFCKNSADNKVVCSCTEG
YRLAENQKSCEPAVPFPCGRVSVSQTSKLTRAETVFPDVD
YVNSTEAETILDNITQSTQSFNDFTRVVGGEDAKPGQFPW
QVVLNGKVDAFCGGSIVNEKWIVTAAHCVETGVKITVVAG
EHNIEETEHTEQKRNVIRIIPHHNYNAAINKYNHDIALLE
LDEPLVLNSYVTPICIADKEYTNIFLKFGSGYVSGWGRVF
NKGRSASVLQYLRVPLVDRATCLRSTKFTIYNNMFCAGFH
EGGRDSCQGDSGGPHVTEVEGTSFLTGIISWGEECAMKGK
YGIYTKVSRYVNWIKEKTKLT

Additionally, the “Padua” mutation was introduced into the An96 and An97 fIX proteins to determine if addition of this mutation might increase the factor IX activity. The Padua mutation is a R384L-R338L substitution in the mature fIX amino sequence that increases fIX activity (“fIX Padua,” see Paolo et al, “X-Linked Thrombophilia with a Mutant Factor IX” N Engl J Med; 361:1671-1675, 2009). The sequences of the An96 and An97 fIX proteins with the Padua mutation (shown in bold underline) are as follows:

An96 fIX Padua
(SEQ ID NO: 17)
MQCLNMIMAESPGLITICLLGYLLSAECTVFLDHENATKI
LNRPKRYNSGKLEEFVRGNLERECIEEKCSFEEAREVFEN
TEKTTEFWKQYVDGDQCESNPCLNGGSCKDDINSYECWCR
FGFEGKNCELDATCSIKNGRCKQFCKKSADNKVVCSCTEG
YRLAEDQKSCEPAVPFPCGRVSVSHTSKKLTRAETIFSNM
DYENSTEAETILDNVTQSTQSFNDFTRVVGGENAKPGQFP
WQVLLNGKIDAFCGGSIINEKWVVTAAHCIEPGVKITVVA
GEHNIEKTEPTEQKRNVIRVIPHHNYNATINKYSHDIALL
ELDKPLTLNSYVTPICIANREYTNIFLKFGSGYVSGWGRV
FNRGRSASILQYLRVPLVDRATCLLSTKFTIYNNMFCAGY
HEGGKDSCQGDSGGPHVTEVEGTSFLTGIISWGEECAMKG
KYGIYTKVSRYVNWIKEKTKLT
An97 fIX Padua
(SEQ ID NO: 18)
MQCLNMIMAESPGLITICLLGYLLSAECTVFLDHENANKI
LNRPKRYNSGKLEEFVRGNLERECIEEKCSFEEAREVFEN
TEKTTEFWKQYVDGDQCESNPCLNGGSCKDDINSYECWCR
FGFEGKNCELDATCSIKNGRCKQFCKKSADNKVVCSCTEG
YRLAEDQKSCEPAVPFPCGRVSVSHTSKLTRAETIFSNMD
YENSTEAETILDNVTQSTQSFNDFTRVVGGENAKPGQFPW
QVLLNGKIDAFCGGSIINEKWVVTAAHCIEPGVKITVVAG
EHNIEKTEPTEQKRNVIRVIPHHNYNATINKYSHDIALLE
LDKPLTLNSYVTPICIADREYTNIFLKFGSGYVSGWGRVF
NRGRSASILQYLRVPLVDRATCLLSTKFTIYNNMFCAGYH
EGGKDSCQGDSGGPHVTEVEGTSFLTGIISWGEECAMKGK
YGIYTKVSRYVNWIKEKTKLT

In SEQ ID NO: 16 (Human fIX), residues 1-28 are the signal peptide (bold, referred to as fIX residues −46 to −18), residues 29-46 are the propeptide (italics, referred to as fIX residues −18 to -1), and residues 47-462 are the mature fIX sequence (referred to as mature fIX residues +1 to 415). In SEQ ID NO: 18, residues 1-28 are the signal peptide (bold ital., referred to as mature fIX residues -46 to -18), residues 29-46 are the propeptide (ital, referred to as fIX residues -18 to -1), and residues 47-461 are the mature fIX sequence (referred to as mature fIX residues +1 to 415). With reference to SEQ ID NO: 16, residues 47-92 are the GLA domain, residues 93-129 are the first EGF-like domain, residues 130-192 are the second EGF-like domain, residues 193-227 are the activation peptide, and residues 228-462 are the catalytic domain Corresponding domains are also present in SEQ ID NOS: 18, 52-57.

The cDNA nucleotide sequence coding for these fIX proteins was optimized by implementing a codon usage bias specific for the human liver cell as compared to naturally occurring nucleotide sequence coding for the corresponding non-codon optimized sequence for a human, for example, using the liver-codon-optimization protocol described in WO 2016/168728. Nucleic acid sequences encoding SEQ ID NO: 16 and SEQ ID NO: 18 that are codon-optimized for expression in liver tissue were generated, and are provided as follows:

An96 fIX Padua
(SEQ ID NO: 26)
ATGCAGTGCCTGAACATGATCATGGCCGAGTCCCCCGGCC
TGATCACCATCTGCCTGCTGGGGTACCTGCTGAGCGCCGA
GTGCACCGTGTTCCTGGACCACGAGAACGCCACCAAGATC
CTGAACAGGCCCAAGAGATACAACTCCGGCAAGCTGGAGG
AGTTCGTGAGGGGGAACCTGGAGAGAGAGTGCATCGAGGA
GAAGTGCAGCTTCGAGGAGGCCAGGGAGGTGTTCGAGAAC
ACCGAGAAGACCACCGAGTTCTGGAAGCAGTACGTGGACG
GCGACCAGTGCGAGTCCAACCCCTGCCTGAACGGCGGGTC
CTGCAAGGACGACATCAACAGCTACGAGTGCTGGTGCAGG
TTCGGCTTCGAGGGGAAGAACTGCGAGCTGGACGCCACCT
GCAGCATCAAGAACGGCAGATGCAAGCAGTTCTGCAAGAA
GTCCGCCGACAACAAGGTGGTGTGCAGCTGCACCGAGGGA
TACAGACTGGCTGAGGACCAGAAGTCCTGCGAGCCAGCTG
TGCCATTCCCATGCGGGAGGGTGTCCGTGAGCCACACCAG
CAAGAAGCTGACCAGAGCCGAAACCATCTTCTCCAACATG
GACTACGAGAACAGCACCGAGGCCGAAACCATCCTGGACA
ACGTGACCCAGTCCACCCAGAGCTTCAACGACTTCACCCG
GGTGGTGGGAGGAGAGAACGCTAAGCCAGGACAGTTCCCA
TGGCAGGTGCTGCTGAACGGGAAGATCGACGCCTTCTGCG
GCGGGTCCATCATCAACGAGAAGTGGGTGGTGACCGCTGC
TCACTGCATCGAGCCAGGAGTGAAGATCACCGTGGTGGCT
GGGGAGCACAACATCGAGAAGACCGAGCCCACCGAGCAGA
AGCGCAACGTGATCCGCGTGATCCCCCACCACAACTACAA
CGCCACCATCAACAAGTACTCCCACGACATCGCCCTGCTG
GAGCTGGACAAGCCCCTGACCCTGAACAGCTACGTGACCC
CCATCTGCATCGCCAACAGGGAGTACACCAACATCTTCCT
GAAGTTCGGATCCGGATACGTGAGCGGATGGGGACGCGTG
TTCAACCGCGGCCGGTCCGCCAGCATCCTGCAGTACCTGA
GAGTGCCACTGGTGGACAGAGCTACCTGCCTGCTGTCCAC
CAAGTTCACCATCTACAACAACATGTTCTGCGCTGGATAC
CACGAGGGAGGGAAGGACTCCTGCCAGGGGGACAGCGGAG
GACCACACGTGACCGAGGTGGAGGGCACCTCCTTCCTGAC
CGGCATCATCAGCTGGGGGGAGGAGTGCGCCATGAAGGGC
AAGTACGGGATCTACACCAAGGTGAGCAGATACGTGAACT
GGATCAAGGAGAAGACCAAGCTGACCTGA
An97 fIX Padua
(SEQ ID NO: 27)
ATGCAGTGCCTGAACATGATCATGGCCGAGTCCCCCGGCC
TGATCACCATCTGCCTGCTGGGGTACCTGCTGAGCGCCGA
GTGCACCGTGTTCCTGGACCACGAGAACGCCAACAAGATC
CTGAACAGGCCCAAGAGA TACAACTCCGGCAAGCTGGAG
GAGTTCGTGAGGGGGAACCTGGAGAGAGAGTGCATCGAGG
AGAAGTGCAGCTTCGAGGAGGCCAGGGAGGTGTTCGAGAA
CACCGAGAAGACCACCGAGTTCTGGAAGCAGTACGTGGAC
GGCGACCAGTGCGAGTCCAACCCCTGCCTGAACGGCGGGT
CCTGCAAGGACGACATCAACAGCTACGAGTGCTGGTGCAG
GTTCGGCTTCGAGGGGAAGAACTGCGAGCTGGACGCCACC
TGCAGCATCAAGAACGGCAGATGCAAGCAGTTCTGCAAGA
AGTCCGCCGACAACAAGGTGGTGTGCAGCTGCACCGAGGG
ATACAGACTGGCTGAGGACCAGAAGTCCTGCGAGCCAGCT
GTGCCATTCCCATGCGGGAGGGTGTCCGTGAGCCACACCA
GCAAGCTGACCAGAGCCGAAACCATCTTCTCCAACATGGA
CTACGAGAACAGCACCGAGGCCGAAACCATCCTGGACAAC
GTGACCCAGTCCACCCAGAGCTTCAACGACTTCACCCGGG
TGGTGGGAGGAGAGAACGCTAAGCCAGGACAGTTCCCATG
GCAGGTGCTGCTGAACGGGAAGATCGACGCCTTCTGCGGC
GGGTCCATCATCAACGAGAAGTGGGTGGTGACCGCTGCTC
ACTGCATCGAGCCAGGAGTGAAGATCACCGTGGTGGCTGG
GGAGCACAACATCGAGAAGACCGAGCCCACCGAGCAGAAG
CGCAACGTGATCCGCGTGATCCCCCACCACAACTACAACG
CCACCATCAACAAGTACTCCCACGACATCGCCCTGCTGGA
GCTGGACAAGCCCCTGACCCTGAACAGCTACGTGACCCCC
ATCTGCATCGCCGACAGGGAGTACACCAACATCTTCCTGA
AGTTCGGATCCGGATACGTGAGCGGATGGGGACGCGTGTT
CAACCGCGGCCGGTCCGCCAGCATCCTGCAGTACCTGAGA
GTGCCACTGGTGGACAGAGCTACCTGCCTGCTGTCCACCA
AGTTCACCATCTACAACAACATGTTCTGCGCTGGATACCA
CGAGGGAGGGAAGGACTCCTGCCAGGGGGACAGCGGAGGA
CCACACGTGACCGAGGTGGAGGGCACCTCCTTCCTGACCG
GCATCATCAGCTGGGGGGAGGAGTGCGCCATGAAGGGCAA
GTACGGGATCTACACCAAGGTGAGCAGATACGTGAACTGG
ATCAAGGAGAAGACCAAGCTGACCTGA

In SEQ ID NOs: 17 and 27, the signal peptide is shown in bold, the propeptide is shown in bold italics, and the mutated nucleotide of the Padua mutation is shown in bold underline. The liver codon-optimized fIX Padua sequences can be included in a vector (such as an AAV vector) and operably linked to a promoter (such as a liver specific promoter, for example, the HCB promoter) for administration to a subject, for example, to treat hemophilia B in the subject.

See FIGS. 1A and 1B for an alignment of the amino acid sequences encoded by the following AN96fIX constructs: An96 fIX (SEQ ID NO: 2), hfIX-96sp3pro (SEQ ID NO: 3), or An96-hfIXpro (SEQ ID NO: 4), or hfIX-96appro (SEQ ID NO: 5), or hfIX-96e2pro (SEQ ID NO: 6), or hfIX-96e2V86A (SEQ ID NO: 7), or hfIX-96e2V86Apro2 (SEQ ID NO: 8), or hfIX-96ge2appro (SEQ ID NO: 9), or hfIX-96ge2pro (SEQ ID NO: 10), or hfIX-96ge2pro2 (SEQ ID NO: 11), or hfIX-96gpro (SEQ ID NO: 12), or hfIX-96pro (SEQ ID NO: 13), or hfIX-96pro2 (SEQ ID NO: 14).

hfIX-96sp3pro hfIX with An96 Signal Peptide, Propetide, and Protease Domains
(SEQ ID NO: 3)
ATGCAGTGCCTGAACATGATCATGGCCGAGTCCCCCGGCCTGATCACCATCTGCCTGCTGGGGTACCTGCTGAGCGCCGA
GTGCACCGTGTTCCTGGACCACGAGAACGCCACCAAGATCCTGAACAGGCCCAAGAGATACAATTCTGGAAAGCTGGAGG
AATTTGTGCAGGGCAACCTGGAGAGGGAATGCATGGAGGAAAAGTGTAGCTTTGAGGAAGCTAGGGAGGTGTTTGAAAAC
ACAGAGAGGACCACAGAATTCTGGAAGCAGTATGTGGATGGAGATCAGTGTGAGTCCAACCCCTGTCTGAATGGAGGGTC
TTGCAAAGATGATATCAACTCCTATGAGTGCTGGTGTCCTTTTGGATTTGAAGGCAAAAATTGTGAGCTGGATGTGACCT
GTAACATCAAGAATGGCAGGTGTGAGCAGTTCTGTAAAAACTCTGCTGATAATAAGGTGGTCTGCAGCTGTACAGAAGGC
TATAGGCTGGCTGAGAACCAGAAGAGCTGTGAACCAGCTGTGCCCTTCCCTTGTGGGAGGGTGTCTGTCAGCCAGACCTC
TAAGCTGACCAGAGCTGAGACTGTGTTCCCAGATGTGGATTATGTCAACTCCACAGAGGCTGAAACCATCCTGGACAACA
TCACCCAGTCTACCCAGTCCTTCAATGACTTTACCAGAGTGGTGGGAGGAGAGAACGCTAAGCCAGGACAGTTCCCATGG
CAGGTGCTGCTGAACGGGAAGATCGACGCCTTCTGCAGGCGGGTCCATCATCACGAGAAGTGGGTGGTGACCGCTGCTCA
CTGCATCGAGCCAGGAGTGAAGATCACCGTGGTGGCTGGGGAGCACAACATCGAGAAGACCGAGCCCACCGAGCAGAAGC
GCAACGTGATCCGCGTGATCCCCCACCACAACTACAACGCCACCATCAACAAGTACTCCCACGACATCGCCCTGCTGGAG
CTGGACAAGCCCCTGACCCTGAACAGCTACGTGACCCCCATCTGCATCGCCAACAGGGAGTACACCAACATCTTCCTGAA
GTTCGGATCCGGATACGTGAGCGGATGGGGACGCGTGTTCAACCGCGGCCGGTCCGCCAGCATCCTGCAGTACCTGAGAG
TGCCACTGGTGGACAGAGCTACCTGCCTGCGGTCCACCAAGTTCACCATCTACAACAACATGTTCTGCGCTGGATACCAC
GAGGGAGGGAAGGACTCCTGCCAGGGGGACAGCGGAGGACCACACGTGACCGAGGTGGAGGGCACCTCCTTCCTGACCGG
CATCATCAGCTGGGGGGAGGAGTGCGCCATGAAGGGCAA

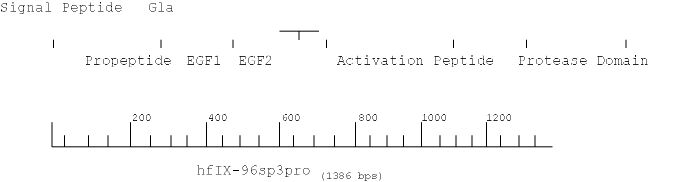

An96-hfIXpro An96 with hfIX Protease Domain
(SEQ ID NO: 4)
ATGCAGTGCCTGAACATGATCATGGCCGAGTCCCCCGGCCTGATCACCATCTGCCTGCTGGGGTACCTGCTGAGCGCCGA
GTGCACCGTGTTCCTGGACCACGAGAACGCCACCAAGATCCTGAACAGGCCCAAGAGATACAACTCCGGCAAGCTGGAGG
AGTTCGTGAGGGGGAACCTGGAGAGAGAGTGCATCGAGGAGAAGTGCAGCTTCGAGGAGGCCAGGGAGGTGTTCGAGAAC
ACCGAGAAGACCACCGAGTTCTGGAAGCAGTACGTGGACGGCGACCAGTGCGAGTCCAACCCCTGCCTGAACGGCGGGTC
CTGCAAGGACGACATCAACAGCTACGAGTGCTGGTGCAGGTTCGGCTTCGAGGGGAAGAACTGCGAGCTGGACGCCACCT
GCAGCATCAAGAACGGCAGATGCAAGCAGTTCTGCAAGAAGTCCGCCGACAACAAGGTGGTGTGCAGCTGCACCGAGGGA
TACAGACTGGCTGAGGACCAGAAGTCCTGCGAGCCAGCTGTGCCATTCCCATGCGGGAGGGTGTCCGTGAGCCACACCAG
CAAGAAGCTGACCAGAGCCGAAACCATCTTCTCCAACATGGACTACGAGAACAGCACCGAGGCCGAAACCATCCTGGACA
ACGTGACCCAGTCCACCCAGAGCTTCAACGACTTCACCCGGGTGGTGGGAGGAGAGGATGCCAAACCAGGCCAGTTCCCC
TGGCAGGTGGTCCTGAATGGGAAGGTGGATGCTTTTTGTGGGGGATCCATTGTGAATGAGAAATGGATTGTCACAGCTGC
TCACTGTGTGGAGACAGGGGTCAAGATCACTGTGGTGGCTGGAGAGCACAACATTGAGGAAACAGAACATACTGAGCAGA
AGAGGAATGTGATCAGAATCATCCCTCACCATAACTACAATGCTGCTATCAACAAATATAATCATGACATTGCCCTGCTG
GAACTGGATGAGCCTCTGGTGCTGAACAGCTATGTCACCCCAATCTGCATTGCTGACAAAGAGTATACCAATATCTTCCT
GAAGTTTGGATCTGGATATGTGTCTGGATGGGGAAGGGTCTTCCACAAGGGCAGGTCTGCCCTGGTGCTGCAGTATCTGA
GGGTGCCTCTGGTGGACAGAGCTACCTGCCTGAGGTCTACCAAGTTCACCATCTACAACAATATGTTCTGTGCTGGATTT
CATGAGGGAGGCAGGGACTCCTGTCAGGGGGATTCTGGAGGCCCACATGTGACAGAGGTGGAAGGCACCAGCTTCCTGAC
TGGCATCATCTCTTGGGGGGAGGAATGTGCTATGAAGGGGAAATATGGAATCTACACCAAAGTGAGCAGGTATGTGAACT
GGATCAAAGAGAAGACCAAACTGACCTGA

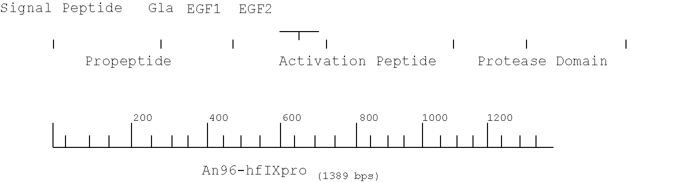

hfIX-96appro hfIX with An96 Activation Peptide and Protease Domains
(SEQ ID NO: 5)
ATGCAGAGGGTCAATATGATCATGGCTGAATCTCCTGGGCTGATCACCATTTGCCTGCTGGGATACCTGCTGTCTGCTGA
GTGTACAGTGTTCCTGGACCATGAGAATGCCAATAAGATCCTGAACAGGCCCAAAAGATACAATTCTGGAAAGCTGGAGG
AATTTGTGCAGGGCAACCTGGAGAGGGAATGCATGGAGGAAAAGTGTAGCTTTGAGGAAGCTAGGGAGGTGTTTGAAAAC
ACAGAGAGGACCACAGAATTCTGGAAGCAGTATGTGGATGGAGATCAGTGTGAGTCCAACCCCTGTCTGAATGGAGGGTC
TTGCAAAGATGATATCAACTCCTATGAGTGCTGGTGTCCTTTTGGATTTGAAGGCAAAAATTGTGAGCTGGATGTGACCT
GTAACATCAAGAATGGCAGGTGTGAGCAGTTCTGTAAAAACTCTGCTGATAATAAGGTGGTCTGCAGCTGTACAGAAGGC
TATAGGCTGGCTGAGAACCAGAAGAGCTGTGAACCAGCTGTGCCCTTCCCTTGTGGGAGGGTGTCTGTCAGCCAGACCTC
TAAGCTGACCAGAGCCGAAACCATCTTCTCCAACATGGACTACGAGAACAGCACCGAGGCCGAAACCATCCTGGACAACG
TGACCCAGTCCACCCAGAGCTTCAACGACTTCACCCGGGTGGTGGGAGGAGAGAACGCTAAGCCAGGACAGTTCCCATGG
CAGGTGCTGCTGAACGGGAAGATCGACGCCTTCTGCGGCGGGTCCATCATCAACGAGAAGTGGGTGGTGACCGCTGCTCA
CTGCATCGAGCCAGGAGTGAAGATCACCGTGGTGGCTGGGGAGCACAACATCGAGAAGACCGAGCCCACCGAGCAGAAGC
GCAACGTGATCCGCGTGATCCCCCACCACAACTACAACGCCACCATCAACAAGTACTCCCACGACATCGCCCTGCTGGAG
CTGGACAAGCCCCTGACCCTGAACAGCTACGTGACCCCCATCTGCATCGCCAACAGGGAGTACACCAACATCTTCCTGAA
GTTCGGATCCGGATACGTGAGCGGATGGGGACGCGTGTTCAACCGCGGCCGGTCCGCCAGCATCCTGCAGTACCTGAGAG
TGCCACTGGTGGACAGAGCTACCTGCCTGCGGTCCACCAAGTTCACCATCTACAACAACATGTTCTGCGCTGGATACCAC
GAGGGAGGGAAGGACTCCTGCCAGGGGGACAGCGGAGGACCACACGTGACCGAGGTGGAGGGCACCTCCTTCCTGACCGG
CATCATCAGCTGGGGGGAGGAGTGCGCCATGAAGGGCAAGTACGGGATCTACACCAAGGTGAGCAGATACGTGAACTGGA
TCAAGGAGAAGACCAAGCTGACCTGA

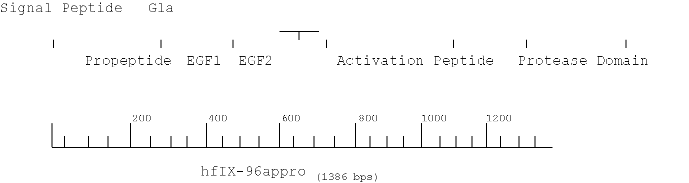

hfIX-96e2pro hfIX with An96 EGF2 and Protease Domains
(SEQ ID NO: 6)
ATGCAGAGGGTCAATATGATCATGGCTGAATCTCCTGGGCTGATCACCATTTGCCTGCTGGGATACCTGCTGTCTGCTGA
GTGTACAGTGTTCCTGGACCATGAGAATGCCAATAAGATCCTGAACAGGCCCAAAAGATACAATTCTGGAAAGCTGGAGG
AATTTGTGCAGGGCAACCTGGAGAGGGAATGCATGGAGGAAAAGTGTAGCTTTGAGGAAGCTAGGGAGGTGTTTGAAAAC
ACAGAGAGGACCACAGAATTCTGGAAGCAGTATGTGGATGGAGATCAGTGTGAGTCCAACCCCTGTCTGAATGGAGGGTC
TTGCAAAGATGATATCAACTCCTATGAGTGCTGGTGTCCTTTTGGATTTGAAGGCAAAAATTTGAGCTGGACGCCACCTG
CAGCATCAAGAACGGCAGATGCAAGCAGTTCTGCAAGAAGTCCGCCGACAACAAGGTGGTGTGCAGCTGCACCGAGGGAT
ACAGACTGGCTGAGGACCAGAAGTCCTGCGAGCCAGCTGTGCCCTTCCCTTGTGGGAGGGTGTCTGTCAGCCAGACCTCT
AAGCTGACCAGAGCTGAGACTGTGTTCCCAGATGTGGATTATGTCAACTCCACAGAGGCTGAAACCATCCTGGACAACAT
CACCCAGTCTACCCAGTCCTTCAATGACTTTACCAGAGTGGTGGGAGGAGAGAACGCTAAGCCAGGACAGTTCCCATGGC
AGGTGCTGCTGAACGGGAAGATCGACGCCTTCTGCGGCGGGTCCATCATCAACGAGAAGTGGGTGGTGACCGCTGCTCAC
TGCATCGAGCCAGGAGTGAAGATCACCGTGGTGGCTGGGGAGCACAACATCGAGAAGACCGAGCCCACCGAGCAGAAGCG
CAACGTGATCCGCGTGATCCCCCACCACAACTACAACGCCACCATCAACAAGTACTCCCACGACATCGCCCTGCTGGAGC
TGGACAAGCCCCTGACCCTGAACAGCTACGTGACCCCCATCTGCATCGCCAACAGGGAGTACACCAACATCTTCCTGAAG
TTCGGATCCGGATACGTGAGCGGATGGGGACGCGTGTTCAACCGCGGCCGGTCCGCCAGCATCCTGCAGTACCTGAGAGT
GCCACTGGTGGACAGAGCTACCTGCCTGCGGTCCACCAAGTTCACCATCTACAACAACATGTTCTGCGCTGGATACCACG
AGGGAGGGAAGGACTCCTGCCAGGGGGACAGCGGAGGACCACACGTGACCGAGGTGGAGGGCACCTCCTTCCTGACCGGC
ATCATCAGCTGGGGGGAGGAGTGCGCCATGAAGGGCAAGTACGGGATCTACACCAAGGTGAGCAGATACGTGAACTGGAT
CAAGGAGAAGACCAAGCTGACCTGA

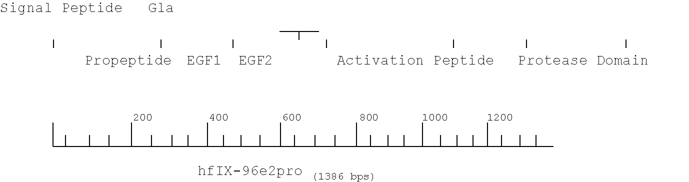

hfIX-96e2V86A hfIX containing EGF2 V86A Amino Acid Substitution from An96
(SEQ ID NO: 7)
ATGCAGAGGGTCAATATGATCATGGCTGAATCTCCTGGGCTGATCACCATTTGCCTGCTGGGATACCTGCTGTCTGCTGA
GTGTACAGTGTTCCTGGACCATGAGAATGCCAATAAGATCCTGAACAGGCCCAAAAGATACAATTCTGGAAAGCTGGAGG
AATTTGTGCAGGGCAACCTGGAGAGGGAATGCATGGAGGAAAAGTGTAGCTTTGAGGAAGCTAGGGAGGTGTTTGAAAAC
ACAGAGAGGACCACAGAATTCTGGAAGCAGTATGTGGATGGAGATCAGTGTGAGTCCAACCCCTGTCTGAATGGAGGGTC
TTGCAAAGATGATATCAACTCCTATGAGTGCTGGTGTCCTTTTGGATTTGAAGGCAAAAATTGTGAGCTGGATGCCACCT
GTAACATCAAGAATGGCAGGTGTGAGCAGTTCTGTAAAAACTCTGCTGATAATAAGGTGGTCTGCAGCTGTACAGAAGGC
TATAGGCTGGCTGAGAACCAGAAGAGCTGTGAACCAGCTGTGCCCTTCCCTTGTGGGAGGGTGTCTGTCAGCCAGACCTC
TAAGCTGACCAGAGCTGAGACTGTGTTCCCAGATGTGGATTATGTCAACTCCACAGAGGCTGAAACCATCCTGGACAACA
TCACCCAGTCTACCCAGTCCTTCAATGACTTTACCAGAGTGGTGGGAGGAGAGGATGCCAAACCAGGCCAGTTCCCCTGG
CAGGTGGTCCTGAATGGGAAGGTGGATGCTTTTTGTGGGGGATCCATTGTGAATGAGAAATGGATTGTCACAGCTGCTCA
CTGTGTGGAGACAGGGGTCAAGATCACTGTGGTGGCTGGAGAGCACAACATTGAGGAAACAGAACATACTGAGCAGAAGA
GGAATGTGATCAGAATCATCCCTCACCATAACTACAATGCTGCTATCAACAAATATAATCATGACATTGCCCTGCTGGAA
CTGGATGAGCCTCTGGTGCTGAACAGCTATGTCACCCCAATCTGCATTGCTGACAAAGAGTATACCAATATCTTCCTGAA
GTTTGGATCTGGATATGTGTCTGGATGGGGAAGGGTCTTCCACAAGGGCAGGTCTGCCCTGGTGCTGCAGTATCTGAGGG
TGCCTCTGGTGGACAGAGCTACCTGCCTGAGGTCTACCAAGTTCACCATCTACAACAATATGTTCTGTGCTGGATTTCAT
GAGGGAGGCAGGGACTCCTGTCAGGGGGATTCTGGAGGCCCACATGTGACAGAGGTGGAAGGCACCAGCTTCCTGACTGG
CATCATCTCTTGGGGGGAGGAATGTGCTATGAAGGGGAAATATGGAATCTACACCAAAGTGAGCAGGTATGTGAACTGGA
TCAAAGAGAAGACCAAACTGACCTGA

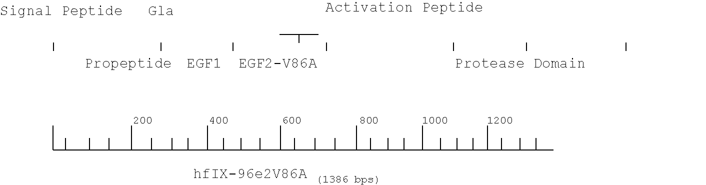

hfIX-96e2V86Apro2 hfIX containing EGF2 V86A Amino Acid Substitution and Pro2
from An96
(SEQ ID NO: 8)
ATGCAGAGGGTCAATATGATCATGGCTGAATCTCCTGGGCTGATCACCATTTGCCTGCTGGGATACCTGCTGTCTGCTGA
GTGTACAGTGTTCCTGGACCATGAGAATGCCAATAAGATCCTGAACAGGCCCAAAAGATACAATTCTGGAAAGCTGGAGG
AATTTGTGCAGGGCAACCTGGAGAGGGAATGCATGGAGGAAAAGTGTAGCTTTGAGGAAGCTAGGGAGGTGTTTGAAAAC
ACAGAGAGGACCACAGAATTCTGGAAGCAGTATGTGGATGGAGATCAGTGTGAGTCCAACCCCTGTCTGAATGGAGGGTC
TTGCAAAGATGATATCAACTCCTATGAGTGCTGGTGTCCTTTTGGATTTGAAGGCAAAAATTGTGAGCTGGATGCCACCT
GTAACATCAAGAATGGCAGGTGTGAGCAGTTCTGTAAAAACTCTGCTGATAATAAGGTGGTCTGCAGCTGTACAGAAGGC
TATAGGCTGGCTGAGAACCAGAAGAGCTGTGAACCAGCTGTGCCCTTCCCTTGTGGGAGGGTGTCTGTCAGCCAGACCTC
TAAGCTGACCAGAGCTGAGACTGTGTTCCCAGATGTGGATTATGTCAACTCCACAGAGGCTGAAACCATCCTGGACAACA
TCACCCAGTCTACCCAGTCCTTCAATGACTTTACCAGAGTGGTGGGAGGAGAGGATGCCAAACCAGGCCAGTTCCCCTGG
CAGGTGGTCCTGAATGGGAAGGTGGATGCTTTTTGTGGGGGATCCATTGTGAATGAGAAATGGATTGTCACAGCTGCTCA
CTGTGTGGAGACAGGGGTCAAGATCACTGTGGTGGCTGGAGAGCACAACATTGAGGAAACAGAACATACTGAGCAGAAGA
GGAATGTGATCAGAATCATCCCTCACCATAACTACAATGCTGCTATCAACAAGTACTCCCACGACATCGCCCTGCTGGGA
CTGGACAAGCCCCTGACCCTGAACAGCTACGTGACCCCCATCTGCATCGCCAACAGGGAGTACACCAACATCTTCCTGAA
GTTCGGATCCGGATACGTGAGCGGATGGGGACGCGTGTTCAACCGCGGCCGGTCCGCCAGCATCCTGCAGTACCTGAGAG
TGCCACTGGTGGACAGAGCTACCTGCCTGCGGTCCACCAAGTTCACCATCTACAACAACATGTTCTGCGCTGGATACCAC
GAGGGAGGGAAGGACTCCTGCCAGGGGGACAGCGGAGGACCACACGTGACCGAGGTGGAGGGCACCTCCTTCCTGACCGG
CATCATCAGCTGGGGGGAGGAGTGCGCCATGAAGGGCAAGTACGGGATCTACACCAAGGTGAGCAGATACGTGAACTGGA
TCAAGGAGAAGACCAAGCTGACCTGA

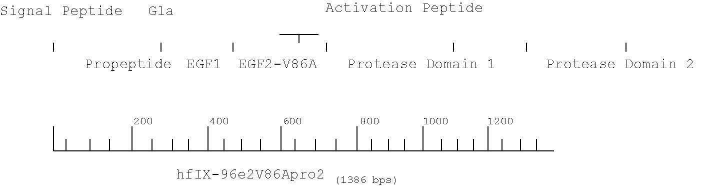

hfIX-96ge2appro hfIX with An96 Gla, EGF2, Activation Peptide, and Protease
Domains
(SEQ ID NO: 9)
ATGCAGAGGGTCAATATGATCATGGCTGAATCTCCTGGGCTGATCACCATTTGCCTGCTGGGATACCTGCTGTCTGCTGA
GTGTACAGTGTTCCTGGACCATGAGAATGCCAATAAGATCCTGAACAGGCCCAAAAGATACAACTCCGGCAAGCTGGAGG
AGTTCGTGAGGGGGAACCTGGAGAGAGAGTGCATCGAGGAGAAGTGCAGCTTCGAGGAGGCCAGGGAGGTGTTCGAGAAC
ACCGAGAAGACCACCGAGTTCTGGAAGCAGTACGTGGATGGAGATCAGTGTGAGTCCAACCCCTGTCTGAATGGAGGGTC
TTGCAAAGATGATATCAACTCCTATGAGTGCTGGTGTCCTTTTGGATTTGAAGGCAAAAATTGTGAGCTGGACGCCACCT
GCAGCATCAAGAACGGCAGATGCAAGCAGTTCTGCAAGAAGTCCGCCGACAACAAGGTGGTGTGCAGCTGCACCGAGGGA
TACAGACTGGCTGAGGACCAGAAGTCCTGCGAGCCAGCTGTGCCCTTCCCTTGTGGGAGGGTGTCTGTCAGCCAGACCTC
TAAGCTGACCAGAGCCGAAACCATCTTCTCCAACATGGACTACGAGAACAGCACCGAGGCCGAAACCATCCTGGACAACG
TGACCCAGTCCACCCAGAGCTTCAACGACTTCACCCGGGTGGTGGGAGGAGAGAACGCTAAGCCAGGACAGTTCCCATGG
CAGGTGCTGCTGAACGGGAAGATCGACGCCTTCTGCGGCGGGTCCATCATCAACGAGAAGTGGGTGGTGACCGCTGCTCA
CTGCATCGAGCCAGGAGTGAAGATCACCGTGGTGGCTGGGGAGCACAACATCGAGAAGACCGAGCCCACCGAGCAGAAGC
GCAACGTGATCCGCGTGATCCCCCACCACAACTACAACGCCACCATCAACAAGTACTCCCACGACATCGCCCTGCTGGAG
CTGGACAAGCCCCTGACCCTGAACAGCTACGTGACCCCCATCTGCATCGCCAACAGGGAGTACACCAACATCTTCCTGAA
GTTCGGATCCGGATACGTGAGCGGATGGGGACGCGTGTTCAACCGCGGCCGGTCCGCCAGCATCCTGCAGTACCTGAGAG
TGCCACTGGTGGACAGAGCTACCTGCCTGCGGTCCACCAAGTTCACCATCTACAACAACATGTTCTGCGCTGGATACCAC
GAGGGAGGGAAGGACTCCTGCCAGGGGGACAGCGGAGGACCACACGTGACCGAGGTGGAGGGCACCTCCTTCCTGACCGG
CATCATCAGCTGGGGGGAGGAGTGCGCCATGAAGGGCAAGTACGGGATCTACACCAAGGTGAGCAGATACGTGAACTGGA
TCAAGGAGAAGACCAAGCTGACCTGA

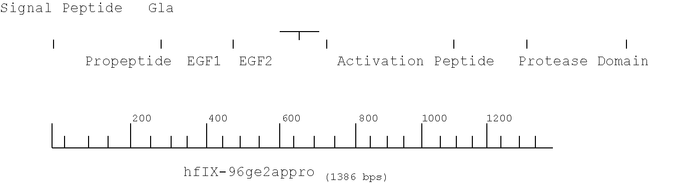

hfIX-96ge2pro hfIX with An96 Gla, EGF2, and Protease Domains
(SEQ ID NO: 10)
ATGCAGAGGGTCAATATGATCATGGCTGAATCTCCTGGGCTGATCACCATTTGCCTGCTGGGATACCTGCTGTCTGCTGA
GTGTACAGTGTTCCTGGACCATGAGAATGCCAATAAGATCCTGAACAGGCCCAAAAGATACAACTCCGGCAAGCTGGAGG
AGTTCGTGAGGGGGAACCTGGAGAGAGAGTGCATCGAGGAGAAGTGCAGCTTCGAGGAGGCCAGGGAGGTGTTCGAGAAC
ACCGAGAAGACCACCGAGTTCTGGAAGCAGTACGTGGATGGAGATCAGTGTGAGTCCAACCCCTGTCTGAATGGAGGGTC
TTGCAAAGATGATATCAACTCCTATGAGTGCTGGTGTCCTTTTGGATTTGAAGGCAAAAATTGTGAGCTGGACGCCACCT
GCAGCATCAAGAACGGCAGATGCAAGCAGTTCTGCAAGAAGTCCGCCGACAACAAGGTGGTGTGCAGCTGCACCGAGGGA
TACAGACTGGCTGAGGACCAGAAGTCCTGCGAGCCAGCTGTGCCCTTCCCTTGTGGGAGGGTGTCTGTCAGCCAGACCTC
TAAGCTGACCAGAGCTGAGACTGTGTTCCCAGATGTGGATTATGTCAACTCCACAGAGGCTGAAACCATCCTGGACAACA
TCACCCAGTCTACCCAGTCCTTCAATGACTTTACCAGAGTGGTGGGAGGAGAGAACGCTAAGCCAGGACAGTTCCCATGG
CAGGTGCTGCTGAACGGGAAGATCGACGCCTTCTGCGGCGGGTCCATCATCAACGAGAAGTGGGTGGTGACCGCTGCTCA
CTGCATCGAGCCAGGAGTGAAGATCACCGTGGTGGCTGGGGAGCACAACATCGAGAAGACCGAGCCCACCGAGCAGAAGC
GCAACGTGATCCGCGTGATCCCCCACCACAACTACAACGCCACCATCAACAAGTACTCCCACGACATCGCCCTGCTGGAG
CTGGACAAGCCCCTGACCCTGAACAGCTACGTGACCCCCATCTGCATCGCCAACAGGGAGTACACCAACATCTTCCTGAA
GTTCGGATCCGGATACGTGAGCGGATGGGGACGCGTGTTCAACCGCGGCCGGTCCGCCAGCATCCTGCAGTACCTGAGAG
TGCCACTGGTGGACAGAGCTACCTGCCTGCGGTCCACCAAGTTCACCATCTACAACAACATGTTCTGCGCTGGATACCAC
GAGGGAGGGAAGGACTCCTGCCAGGGGGACAGCGGAGGACCACACGTGACCGAGGTGGAGGGCACCTCCTTCCTGACCGG
CATCATCAGCTGGGGGGAGGAGTGCGCCATGAAGGGCAAGTACGGGATCTACACCAAGGTGAGCAGATACGTGAACTGGA
TCAAGGAGAAGACCAAGCTGACCTGA

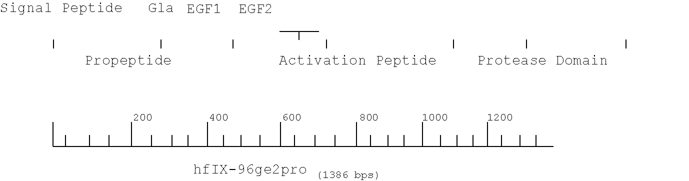

hfIX-96ge2pro2 hfIX containing An96 Gla, EGF2, and Pro2 Domains
(SEQ ID NO: 11)
ATGCAGAGGGTCAATATGATCATGGCTGAATCTCCTGGGCTGATCACCATTTGCCTGCTGGGATACCTGCTGTCTGCTGA
GTGTACAGTGTTCCTGGACCATGAGAATGCCAATAAGATCCTGAACAGGCCCAAAAGATACAACTCCGGCAAGCTGGAGG
AGTTCGTGAGGGGGAACCTGGAGAGAGAGTGCATCGAGGAGAAGTGCAGCTTCGAGGAGGCCAGGGAGGTGTTCGAGAAC
ACCGAGAAGACCACCGAGTTCTGGAAGCAGTACGTGGATGGAGATCAGTGTGAGTCCAACCCCTGTCTGAATGGAGGGTC
TTGCAAAGATGATATCAACTCCTATGAGTGCTGGTGTCCTTTTGGATTTGAAGGCAAAAATTGTGAGCTGGACGCCACCT
GCAGCATCAAGAACGGCAGATGCAAGCAGTTCTGCAAGAAGTCCGCCGACAACAAGGTGGTGTGCAGCTGCACCGAGGGA
TACAGACTGGCTGAGGACCAGAAGTCCTGCGAGCCAGCTGTGCCCTTCCCTTGTGGGAGGGTGTCTGTCAGCCAGACCTC
TAAGCTGACCAGAGCTGAGACTGTGTTCCCAGATGTGGATTATGTCAACTCCACAGAGGCTGAAACCATCCTGGCAACAA
TCACCCAGTCTACCCAGTCCTTCAATGACTTTACCAGAGTGGTGGGAGGAGAGGATGCCAAACCAGGCCAGTTCCCCTGG
CAGGTGGTCCTGAATGGGAAGGTGGATGCTTTTTGTGGGGGATCCATTGTGAATGAGAAATGGATTGTCACAGCTGCTCA
CTGTGTGGAGACAGGGGTCAAGATCACTGTGGTGGCTGGAGAGCACAACATTGAGGAAACAGAACATACTGAGCAGAAGA
GGAATGTGATCAGAATCATCCCTCACCATAACTACAATGCTGCTATCAACAAGTACTCCCACGACATCGCCCTGCTGGAG
CTGGACAAGCCCCTGACCCTGAACAGCTACGTGACCCCCATCTGCATCGCCAACAGGGAGTACACCAACATCTTCCTGAA
GTTCGGATCCGGATACGTGAGCGGATGGGGACGCGTGTTCAACCGCGGCCGGTCCGCCAGCATCCTGCAGTACCTGAGAG
TGCCACTGGTGGACAGAGCTACCTGCCTGCGGTCCACCAAGTTCACCATCTACAACAACATGTTCTGCGCTGGATACCAC
GAGGGAGGGAAGGACTCCTGCCAGGGGGACAGCGGAGGACCACACGTGACCGAGGTGGAGGGCACCTCCTTCCTGACCGG
CATCATCAGCTGGGGGGAGGAGTGCGCCATGAAGGGCAAGTACGGGATCTACACCAAGGTGAGCAGATACGTGAACTGGA
TCAAGGAGAAGACCAAGCTGACCTGA

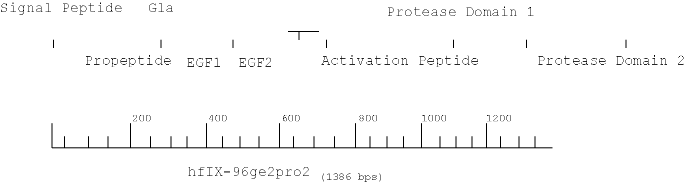

hfIX-96gpro hfIX with An96 Gla and Protease Domains
(SEQ ID NO: 12)
ATGCAGAGGGTCAATATGATCATGGCTGAATCTCCTGGGCTGATCACCATTTGCCTGCTGGGATACCTGCTGTCTGCTGA
GTGTACAGTGTTCCTGGACCATGAGAATGCCAATAAGATCCTGAACAGGCCCAAAAGATACAACTCCGGCAAGCTGGAGG
AGTTCGTGAGGGGGAACCTGGAGAGAGAGTGCATCGAGGAGAAGTGCAGCTTCGAGGAGGCCAGGGAGGTGTTCGAGAAC
ACCGAGAAGACCACCGAGTTCTGGAAGCAGTACGTGGATGGAGATCAGTGTGAGTCCAACCCCTGTCTGAATGGAGGGTC
TTGCAAAGATGATATCAACTCCTATGAGTGCTGGTGTCCTTTTGGATTTGAAGGCAAAAATTGTGAGCTGGATGTGACCT
GTAACATCAAGAATGGCAGGTGTGAGCAGTTCTGTAAAAACTCTGCTGATAATAAGGTGGTCTGCAGCTGTACAGAAGGC
TATAGGCTGGCTGAGAACCAGAAGAGCTGTGAACCAGCTGTGCCCTTCCCTTGTGGGAGGGTGTCTGTCAGCCAGACCTC
TAAGCTGACCAGAGCTGAGACTGTGTTCCCAGATGTGGATTATGTCAACTCCACAGAGGCTGAAACCATCCTGGACAACA
TCACCCAGTCTACCCAGTCCTTCAATGACTTTACCAGAGTGGTGGGAGGAGAGAACGCTAAGCCAGGACAGTTCCCATGG
CAGGTGCTGCTGAACGGGAAGATCGACGCCTTCTGCGGCGGGTCCATCATCAACGAGAAGTGGGTGGTGACCGCTGCTCA
CTGCATCGAGCCAGGAGTGAAGATCACCGTGGTGGCTGGGGAGCACAACATCGAGAAGACCGAGCCCACCGAGCAGAAGC
GCAACGTGATCCGCGTGATCCCCCACCACAACTACAACGCCACCATCAACAAGTACTCCCACGACATCGCCCTGCTGGAG
CTGGACAAGCCCCTGACCCTGAACAGCTACGTGACCCCCATCTGCATCGCCAACAGGGAGTACACCAACATCTTCCTGAA
GTTCGGATCCGGATACGTGAGCGGATGGGGACGCGTGTTCAACCGCGGCCGGTCCGCCAGCATCCTGCAGTACCTGAGAG
TGCCACTGGTGGACAGAGCTACCTGCCTGCGGTCCACCAAGTTCACCATCTACAACAACATGTTCTGCGCTGGATACCAC
GAGGGAGGGAAGGACTCCTGCCAGGGGGACAGCGGAGGACCACACGTGACCGAGGTGGAGGGCACCTCCTTCCTGACCGG
CATCATCAGCTGGGGGGAGGAGTGCGCCATGAAGGGCAAGTACGGGATCTACACCAAGGTGAGCAGATACGTGAACTGGA
TCAAGGAGAAGACCAAGCTGACCTGA

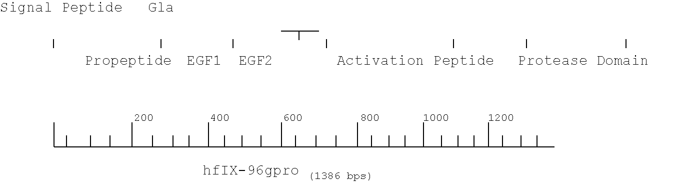

hfIX-96pro hfIX with An96 Protease Domain
(SEQ ID NO: 13)
ATGCAGAGGGTCAATATGATCATGGCTGAATCTCCTGGGCTGATCACCATTTGCCTGCTGGGATACCTGCTGTCTGCTGA
GTGTACAGTGTTCCTGGACCATGAGAATGCCAATAAGATCCTGAACAGGCCCAAAAGATACAATTCTGGAAAGCTGGAGG
AATTTGTGCAGGGCAACCTGGAGAGGGAATGCATGGAGGAAAAGTGTAGCTTTGAGGAAGCTAGGGAGGTGTTTGAAAAC
ACAGAGAGGACCACAGAATTCTGGAAGCAGTATGTGGATGGAGATCAGTGTGAGTCCAACCCCTGTCTGAATGGAGGGTC
TTGCAAAGATGATATCAACTCCTATGAGTGCTGGTGTCCTTTTGGATTTGAAGGCAAAAATTGTGAGCTGGATGTGACCT
GTAACATCAAGAATGGCAGGTGTGAGCAGTTCTGTAAAAACTCTGCTGATAATAAGGTGGTCTGCAGCTGTACAGAAGGC
TATAGGCTGGCTGAGAACCAGAAGAGCTGTGAACCAGCTGTGCCCTTCCCTTGTGGGAGGGTGTCTGTCAGCCAGACCTC
TAAGCTGACCAGAGCTGAGACTGTGTTCCCAGATGTGGATTATGTCAACTCCACAGAGGCTGAAACCATCCTGGACAACA
TCACCCAGTCTACCCAGTCCTTCAATGACTTTACCAGAGTGGTGGGAGGAGAGAACGCTAAGCCAGGACAGTTCCCATGG
CAGGTGCTGCTGAACGGGAAGATCGACGCCTTCTGCGGCGGGTCCATCATCAACGAGAAGTGGGTGGTGACCGCTGCTCA
CTGCATCGAGCCAGGAGTGAAGATCACCGTGGTGGCTGGGGAGCACAACATCGAGAAGACCGAGCCCACCGAGCAGAAGC
GCAACGTGATCCGCGTGATCCCCCACCACAACTACAACGCCACCATCAACAAGTACTCCCACGACATCGCCCTGCTGGAG
CTGGACAAGCCCCTGACCCTGAACAGCTACGTGACCCCCATCTGCATCGCCAACAGGGAGTACACCAACATCTTCCTGAA
GTTCGGATCCGGATACGTGAGCGGATGGGGACGCGTGTTCAACCGCGGCCGGTCCGCCAGCATCCTGCAGTACCTGAGAG
TGCCACTGGTGGACAGAGCTACCTGCCTGCGGTCCACCAAGTTCACCATCTACAACAACATGTTCTGCGCTGGATACCAC
GAGGGAGGGAAGGACTCCTGCCAGGGGGACAGCGGAGGACCACACGTGACCGAGGTGGAGGGCACCTCCTTCCTGACCGG
CATCATCAGCTGGGGGGAGGAGTGCGCCATGAAGGGCAAGTACGGGATCTACACCAAGGTGAGCAGATACGTGAACTGGA
TCAAGGAGAAGACCAAGCTGACCTGA

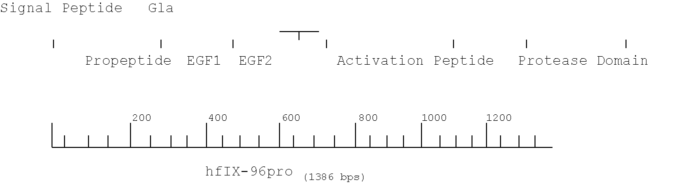

hfIX-96pro2 hfIX with An96 second half of Protease Domain
(SEQ ID NO: 14)
ATGCAGAGGGTCAATATGATCATGGCTGAATCTCCTGGGCTGATCACCATTTGCCTGCTGGGATACCTGCTGTCTGCTGA
GTGTACAGTGTTCCTGGACCATGAGAATGCCAATAAGATCCTGAACAGGCCCAAAAGATACAATTCTGGAAAGCTGGAGG
AATTTGTGCAGGGCAACCTGGAGAGGGAATGCATGGAGGAAAAGTGTAGCTTTGAGGAAGCTAGGGAGGTGTTTGAAAAC
ACAGAGAGGACCACAGAATTCTGGAAGCAGTATGTGGATGGAGATCAGTGTGAGTCCAACCCCTGTCTGAATGGAGGGTC
TTGCAAAGATGATATCAACTCCTATGAGTGCTGGTGTCCTTTTGGATTTGAAGGCAAAAATTGTGAGCTGGATGTGACCT
GTAACATCAAGAATGGCAGGTGTGAGCAGTTCTGTAAAAACTCTGCTGATAATAAGGTGGTCTGCAGCTGTACAGAGGCA
TATAGGCTGGCTGAGAACCAGAAGAGCTGTGAACCAGCTGTGCCCTTCCCTTGTGGGAGGGTGTCTGTCAGCCAGACCTC
TAAGCTGACCAGAGCTGAGACTGTGTTCCCAGATGTGGATTATGTCAACTCCACAGAGGCTGAAACCATCCTGGACAACA
TCACCCAGTCTACCCAGTCCTTCAATGACTTTACCAGAGTGGTGGGAGGAGAGGATGCCAAACCAGGCCAGTTCCCCTGG
CAGGTGGTCCTGAATGGGAAGGTGGATGCTTTTTGTGGGGGATCCATTGTGAATGAGAAATGGATTGTCACAGCTGCTCA
CTGTGTGGAGACAGGGGTCAAGATCACTGTGGTGGCTGGAGAGCACAACATTGAGGAAACAGAACATACTGAGCAGAAGA
GGAATGTGATCAGAATCATCCCTCACCATAACTACAATGCTGCTATCAACAAGTACTCCCACGACATCGCCCTGCTGGAG
CTGGACAAGCCCCTGACCCTGAACAGCTACGTGACCCCCATCTGCATCGCCAACAGGGAGTACACCAACATCTTCCTGAA
GTTCGGATCCGGATACGTGAGCGGATGGGGACGCGTGTTCAACCGCGGCCGGTCCGCCAGCATCCTGCAGTACCTGAGAG
TGCCACTGGTGGACAGAGCTACCTGCCTGCGGTCCACCAAGTTCACCATCTACAACAACATGTTCTGCGCTGGATACCAC
GAGGGAGGGAAGGACTCCTGCCAGGGGGACAGCGGAGGACCACACGTGACCGAGGTGGAGGGCACCTCCTTCCTGACCGG
CATCATCAGCTGGGGGGAGGAGTGCGCCATGAAGGGCAAGTACGGGATCTACACCAAGGTGAGCAGATACGTGAACTGGA
TCAAGGAGAAGACCAAGCTGACCTGA

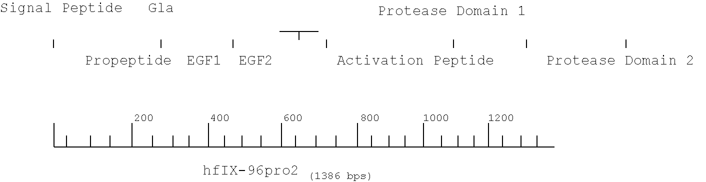

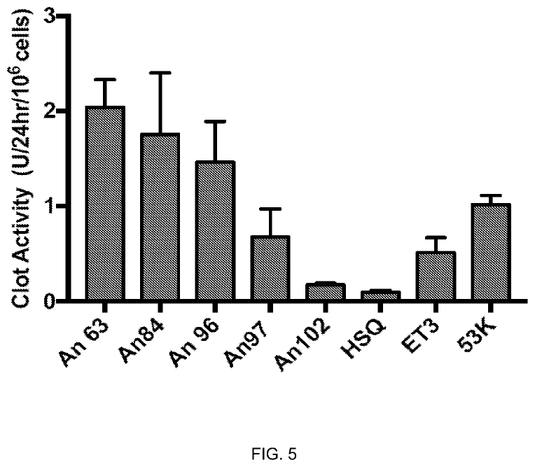

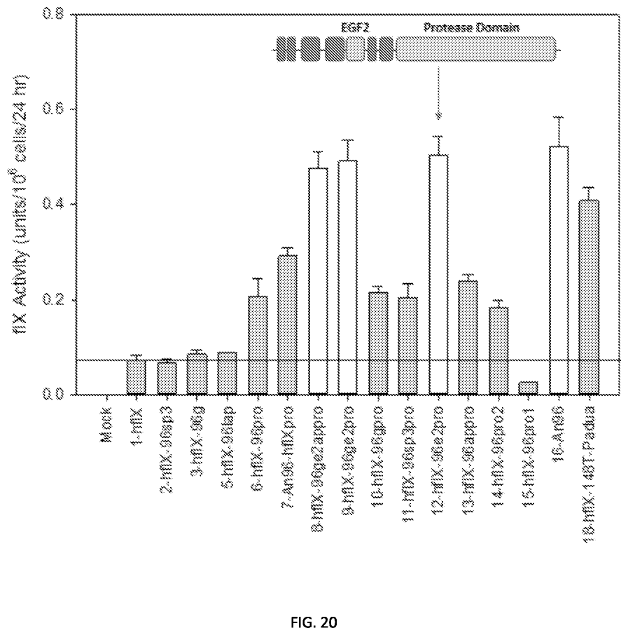

Substitutions are based on ancestral fIX sequences. Ancestral fIX sequences were identified through ASR and synthesized de novo for in vitro expression studies. Ancestral sequence 96 (An96) was identified to have greater activity than human fIX and comparable activity to flX-Padua (R338L). An96 was identified to have greater expression than human fIX (FIG. 6). An96 is 90% human at the amino acid level. Further humanization of An96 while retaining the comparable flX activity and expression through domain swapping studies between human flX and An96. Domains that confer greater activity were identified to be the EGF2 and protease domains of An96. Briefly, constructs were generated by domain swapping and cloned into AAV2 expression plasmids. Huh-7 liver cells were transiently transfected with each construct, and expression of flX measured from conditioned medium by one-stage APTT-dependent clotting assay. Activity was normalized to hfIX expression levels and compared to An96 expression levels. In a series of reiterative experiments, the EGF2 and protease domains of An96 were identified to confer enhanced levels of flX expression when substituted into hfIX. See FIG. 20.

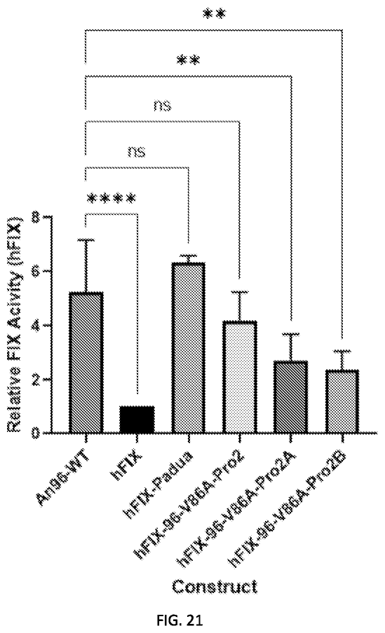

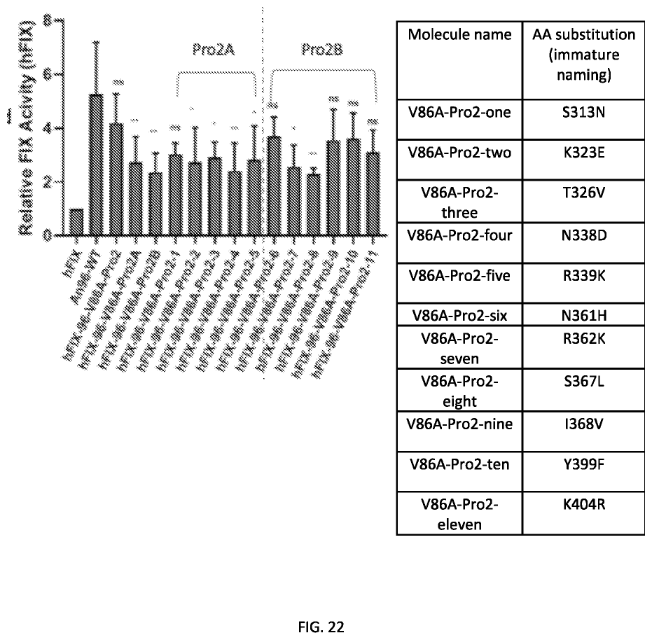

Additional studies showed that only the V132A-V86A substitution from the EGF2 domain and the C-terminal half of the protease domain were required to maintain the high activity. Turning to FIG. 21, Minimization of An96 Protease Domain, fIX expression data here shows that both the Pro2A and 2B regions contain amino acids that are required for high fIX activity. Turning to FIG. 22, Starting with the hFIX-V86A-Pro2 construct, one amino acid at a time was reverted back to the human amino acid, resulting in determination of 4 candidate amino acids from the Pro2A region and just 2 from the Pro2B region.

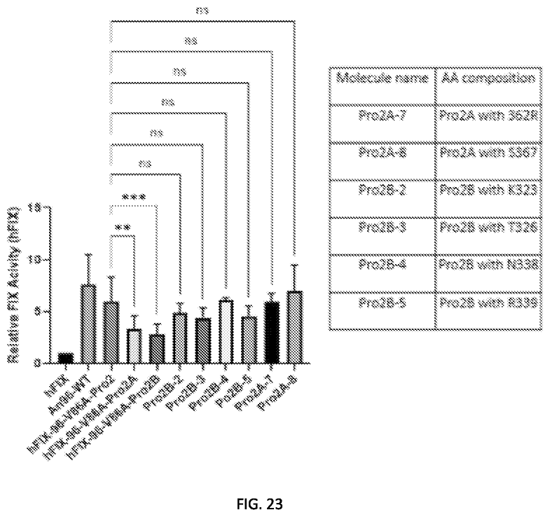

Turning to FIG. 23, Starting with the hFIX-V86A-Pro2A or Pro2B construct, one selected ancestral amino acid at a time was cloned back into the parent molecule.

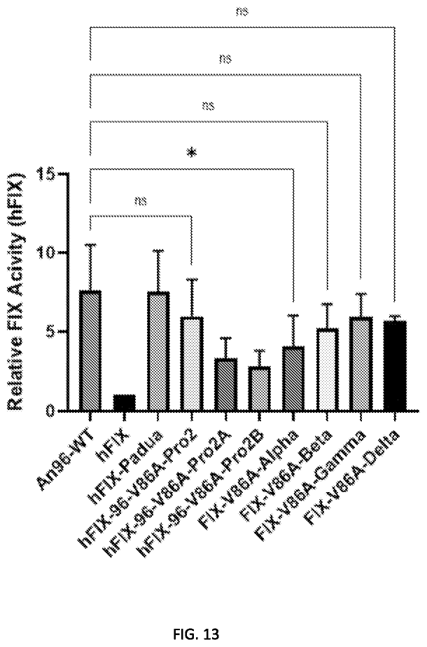

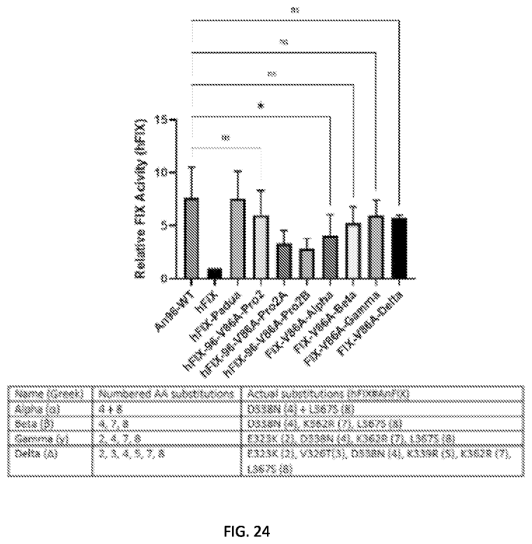

Turning to FIG. 24, After domain swapping, the sequences were further humanized and refined through amino acid substitution studies, through which it was found that the V132A-V86A single point mutation in the EGF2 domain and the N313S-N267S; E323K-E277K; V326T-V280T; D338N-D292N; K339R-K293R; H361N-H315N; K362R-K316R; L367S-L321S; L366S-L320S; V368I-V322I; V367I-V321I; F399Y-F353Y; R404K-R358K; D232N-D186N; V243L-V197L; V248I-V242I; V257I-V211I; I262V-I216V; V269I-V223I; T271P-T225P; E286K-E240K; H289P-H243P; I299V-I253V; A308T-A262T; R384E-R338E or R384L-R338L mutations in the protease domain confer ˜10-fold fIX activity compared to human fIX equivalent to that of An96. As discussed above, various iterations were created and tested and the highest performing constructs, are represented by SEQ ID NOS: 52-57. The fIX transgene(s) are cloned into AAV-expression plasmid containing desired ITR and AAV is manufactured for liver-directed in vivo delivery Finalized constructs with selected combinations of relevant amino acids are shown in FIG. 24. Here, every construct is in a background of hFIX with V86A and the additional noted ancestral amino acids. Turning back to FIG. 14, addition of Padua mutation to minimized An-FIX constructs (e.g., SEQ ID NOS: 56 (Gamma with Padua) and 57 (Beta with Padua)), had an additive effect. FIX Alpah, Beta, Delta and fIX Gamma (+Padua for each of Gamma and Beta) were liver codon optimized to optimize liver codon adaptation indec and minimize mRNA free energy. Four cDNA sequences were selected for each (as discussed below in more detail).

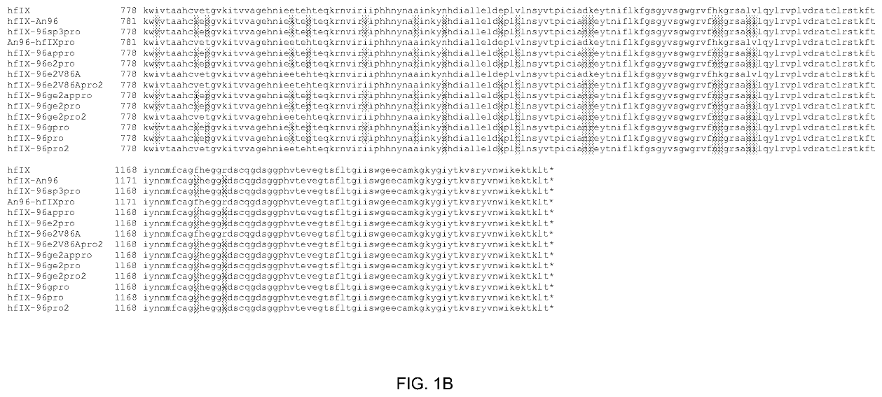

In vitro expression of the optimized fIX sequences was assessed in HepG2 cells transiently transfected with corresponding fIX expression vectors (see FIG. 2). HepG2 cells were seeded at 300,000 cells per well in a 24-well plate containing DMEM supplemented with 10% FBS and 1% Pen/Strep. The cells were approximately 70-80% confluent on the day of transfection. Transfection complex mixtures were prepared at a final concentration of: 0.5 μg plasmid DNA, 1.5 μl TransIT-X2 transfection reagent and OptiMEM supplemented up to a final volume of 50 μL. All of the An-fIX transgenes were cloned into a self-inactivating lentiviral vector expression cassette containing an internal EFla promoter driving An-fIX expression. The respective fIX construct was expressed from a scAAV3 ITR cassette containing the HHS4 enhancer-transthyretin promoter and minute virus of mice intron prior to the human fIX transgene. Transfection complexes were pipetted up and down to mix and allowed to incubate for 15-30 min at room temperature prior to addition dropwise onto the plated cells and gently rocking for even distribution. Media change to DMEM supplemented with 10% FBS and 1% Pen/Strep was performed 24 hr later and the conditioned media was assayed for fIX activity using a one-stage coagulation assay. Each An-fIX protein displayed activity in coagulation assays utilizing human hemophilia B plasma as a substrate thus demonstrating evolutionary mammalian compatibility. As shown in FIG. 2, incorporating the Padua mutation into the An96 and An97 sequences substantially increased the fIX activity relative to corresponding unmodified An96 and An97 proteins. Additionally, the An96 Padua and An97 Padua proteins provided substantially more fIX activity than the human fIX protein (hfIX), which was also encoded by a liver-codon-optimized sequence (˜3.7 fold increase).

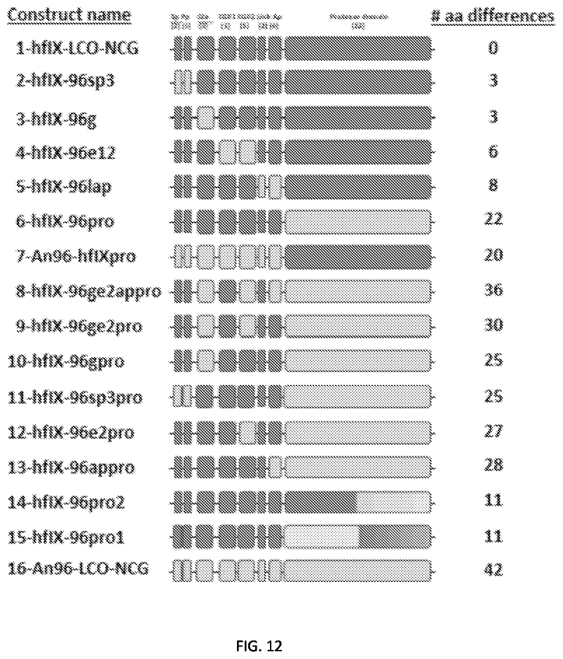

As shown in FIG. 13, incorporating mutations EGF2 (V132A-V86A); N313S-N267S; E323K-E277K; V326T-V280T; D338N-D292N; K339R-K293R; H361N-H315N; K362R-K316R; L367S-L321S; L366S-L320S; V368I-V322I; V367I-V321I; F399Y-F353Y; R404K- R358K; D232N-D186N; V243L-V197L; V248I-V242I; V257I-V211I; I262V-I216V; V269I-V223I; T271P-T225P; E286K-E240K; H289P-H243P; I299V-I253V; A308T-A262T; R384E-R338E or R384L-R338L into sequences substantially increased the fIX activity relative to corresponding unmodified hfIX proteins and closely approximated the fIX activity of hFIX Padua and An96-WT. On FIG. 13, FIX-V86A-Alpha is EGF2 (V132A-V86A): D338N- D292N and L367S-L321S (SEQ ID No. 52, flX Alpha); FIX-V86A-Beta is EGF2 (V132A-V86A); D338N- D292N; K362R- K316R; and L367S-L321S (SEQ ID No. 53, fIX Beta); FIX-V86A-Delta is EGF2 (V132A-V86A); E323K-E277K; V326T-V280T; D338N-D292N; K339R-K293R; K362R-K316R; and L367S-L321S (SEQ ID No. 54, flX Delta); FIX-V86A-Gamma is EGF2 (V132A-V86A); E323K-E277K; D338N-D292N; K362R- K316R; and L367S-L321S (SEQ ID No. 55, flX Gamma). Additionally, the proteins represented by SEQ ID NOS: 52-57 provided substantially more flX activity than the human fIX protein (hfIX), which was also encoded by a liver-codon-optimized sequence (-3.7 to ˜5.0 or more fold increase).

As shown in FIG. 14, incorporating the Padua mutation into the Beta (SEQ ID NO: 57), and Gamma (SEQ ID NO: 56) sequences substantially increased the flX activity relative to corresponding unmodified Alpha, Beta, Delta, Gamma proteins. Additionally, the SEQ ID NOS: 56 and 57 proteins provided substantially more flX activity than the human fIX protein (hfIX), which was also encoded by a liver-codon-optimized sequence (˜3.7 to ˜10 fold to ˜20 fold increase over hfIX).

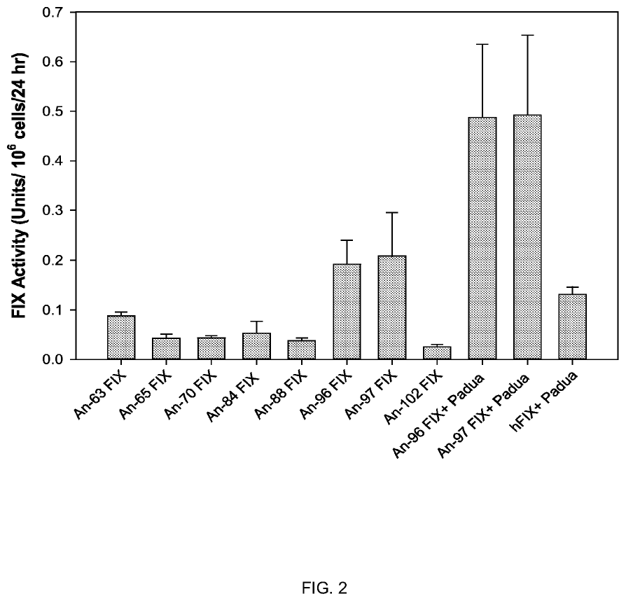

Additionally, in vivo expression of the optimized flX sequences was assessed in hemophilia B mice (FIG. 3). Liver codon optimized human fIX Padua (R338L), An96 fIX, and An96 fIX Padua (R338L, SEQ ID NO: 17) transgenes were cloned into a scAAV3-ITR-HHS4-TTR-MVM-FIX-sPa recombinant AAV expression cassette containing plasmid. The plasmids were linearized with enzymes that preserved ITRS flanking transgene and heat inactivated at 65° C. for 20 minutes. Each digest was screened for DNA quality comparison and shown to be acceptable prior to injection. Mice randomized and plasmid DNA dilutions were made at 5 μg/mL, using TransIT®-BE Delivery Solution warmed to 37° C. Each experimental animal received 0.5 μg linearized plasmid DNA delivered in hydrodynamic fashion in ≤8 s. The injections were performed in a blinded fashion for the 3 treatment groups: 1) scAAV3-HHS4-TTR-MVM-fIX_An96-LCO-sPa, 2) scAAV3-HHS4-TTR-MVM-fIX_An96-Padua-LCO-sPa, 3) scAAV3-HHS4-TTR-MVM-fIX-148T-Padua-LCO-NCO-sPA, as well as a forth control saline-only injection group. A total of 15 experimental mice was used ranging from 9-11 weeks old. Each treatment group received 5 mice. Three 12 week old hemophilia A E16 mice were selected as controls. Mice were ear punched and weighed the day before. Mice were bled 1, 3, 7, and 14 days post plasmid administration. Plasma processed and analyzed for fIX activity using a one-stage coagulation assay. Animals treated with the An96 fIX Padua vector, but not An96 fIX or hfIX treated animals, achieved sustained, supraphysiologic plasma fIX activity levels over two weeks (-15-20 IU/ml fIX activity versus 0-10 IU/ml fIX activity, respectively).

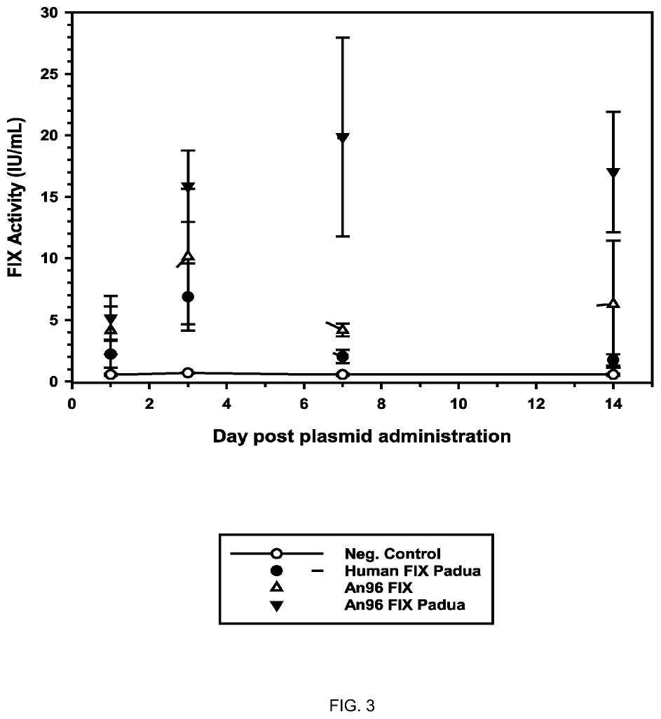

Additionally, in vivo expression of the optimized flX sequences was assessed in fIX^{+/+} mice (FIG. 4). AAV2/8 vectors containing a liver-directed promoter (HCB), minute virus of mouse intron and one of three fIX transgenes (human fIX-Padua), An96-flX-Padua (SEQ ID NO: 17) or hfIX Q11R-E240K-H243P-R338L were produced. The assay was conducted in a blinded fashion on randomized wt fIX^{+/+} mice. Male, 9-11 week old wt fIX^{+/+} mice were injected via tail vein with 5×10¹¹ vector genomes/kg of recombinant AAV (n=3/group). At baseline, prior to AAV administration, and at 4 weeks post-AAV administration, the mice were bled and plasma fIX activity levels were assessed by one-stage coagulation assay. The change in fIX activity level pre- and post-AAV administration is plotted in FIG. 4. Statistical comparisons were made by one-way ANOVA and Holm-Sidak post-hoc analysis. Asterisks denote P<0.05. Mice treated with AAV-2/8-AN96-fIX-PAgua displayed significantly greater increases in fIX activity than control, MX-Padua or hfIX Q11R-E240K-H243P-R338L mice. No other groups were significantly different form each other.

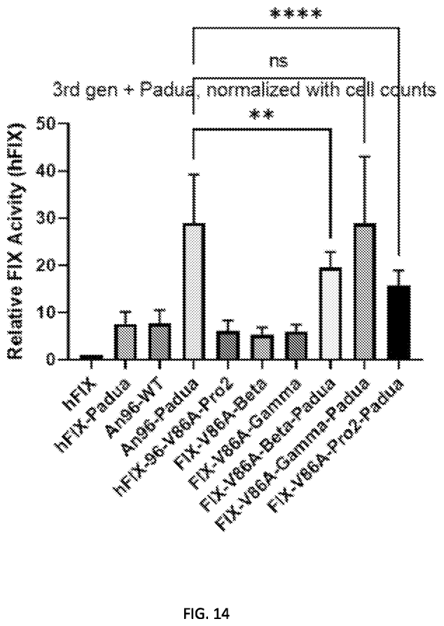

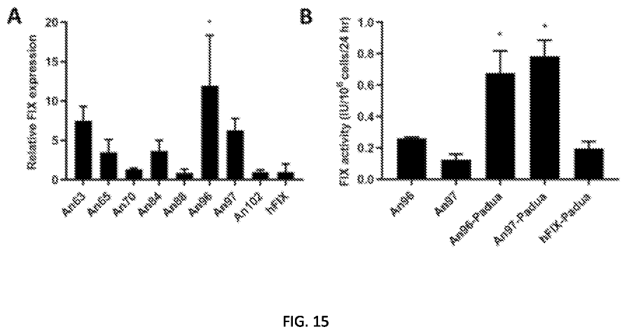

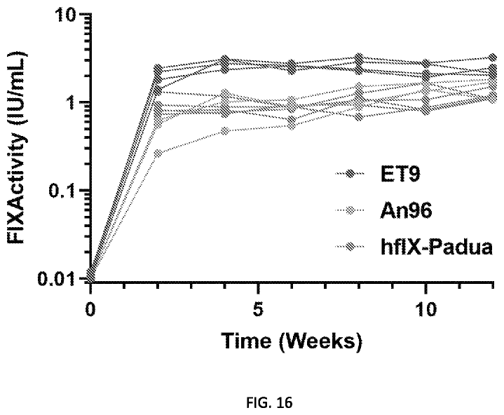

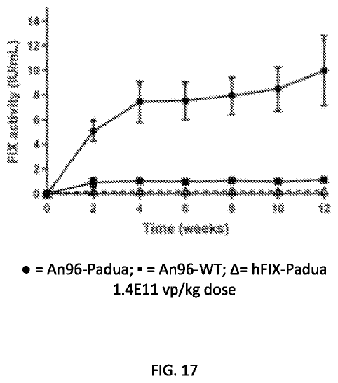

FIGS. 15A, 15B, show further findings of expression of fIX variation in vitro. FIGS. 16 and 17 show further findings of expression of fIX variations in a hemophilia B mouse model, liver-directed AAV gene therapy.

It appears that fIX-An96 and the variants thereon disclosed herein as SEQ ID NOS: 52-57 and reh respective codon optimized nucleic acid sequences, exhibits higher fIX activity due to improved fVIIIa bincing and more efficient production of fXa from fX.

FIG. 16 demonstrates in vivo validation of the variants disclosed herein. Here, an AAV2/8 system was used for murine hemophilia B efficacy studies (5e11vg/g). SEQ ID NO. 55 (referred to hereon as ET9 or V86A+Pro2) showed comparable or superior activity to An96 and/or hfIX-Padua.

As discussed herein, the constructs of SEQ ID NOS. 52-57 are derived from An96 by a series of point mutations and swapping experiments. Performance of each of the resulting sequences represented by SEQ ID NOS. 52-57 are shown to be comparable to An96. Therefore, it follows that each of SEQ ID NOS. 52-57 will have activity and perform in the animal models similar to An96.

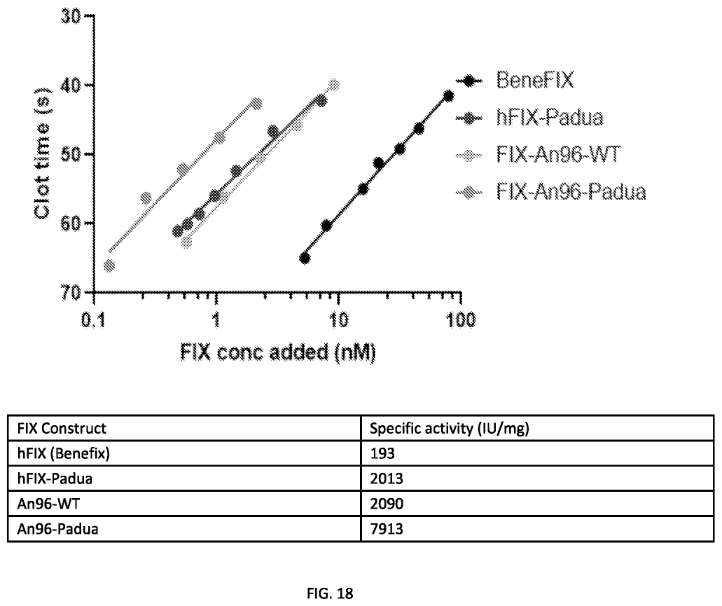

Turning to FIG. 18, at comparable concentrations of fIX protein, hfIX-Padua and An96-WT had similar fibrin clot formation, whereas An96-Padua had greater fibrin clot formation at lower concentrations compared to all other proteins.

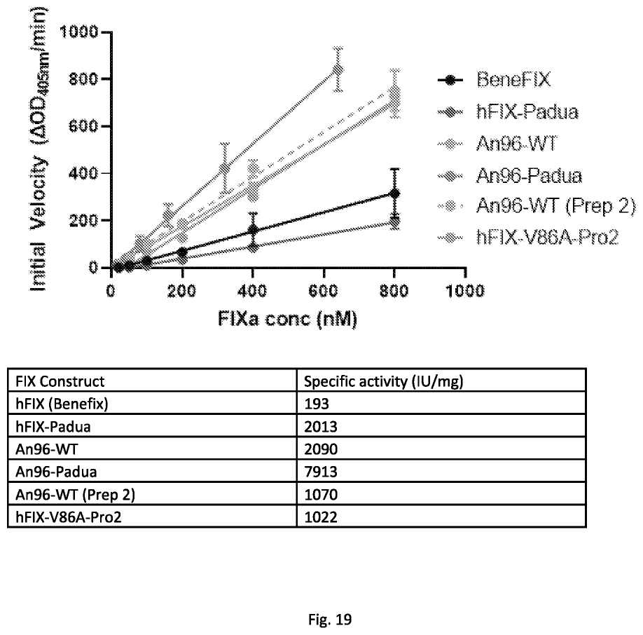

Turning to FIG. 19, An96-WT and An96-Padua have improved enzyme kinetics compared to hFIX. Using a Using a synthetic FIXa peptide substrate (Spectrozyme FIXa), FIXa concentrations ranging from 20-800 nM were incubated with the substrate (1 mM) and change in OD at 405 nm measured. Increased initial reaction velocity for An96, An96-Padua and a partially minimized FIX (hFIX-V86A-Pro2).

Identification of Amino Acid Substitutions in Coagulation fIX that Enhance Activity

Further humanization of An96 revealed the minimum An96 amino acid substitutions required to retain the comparable fIX activity and expression. The humanization occurred through further domain swapping studies between human fIX and An96 as outlined herein. We performed further dissection of the domains that were identified to confer greater activity, the EGF2 and protease domains of An96. The following studies are illustrative.

Turning to FIG. 12, to humanize fIX-An96, domain swapping studies with human fIX were performed. Candidate constructs illustrated in FIG. 12 were designed and cloned into AAV2 expression vectors. Huh-7 liver cells were transiently transfected with equal amounts of AAV2 expression plasmids containing fIX constructs, 24 hours post-transfection, medium was exchanged to AIM-V serum reduced medium, and fIX expression measured by one-stage APTT-dependent coagulation assay from conditioned medium 24 hours after AIM-V medium exchange. FIX activity (units/24 hr/10⁶ cells) was compared to human fIX and An96.

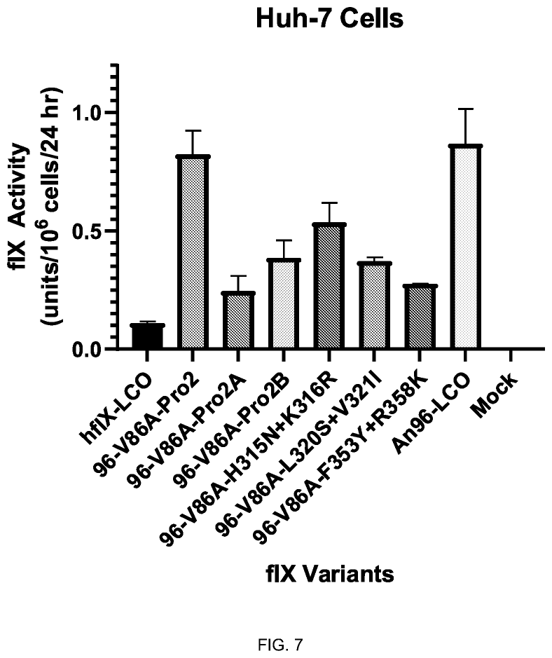

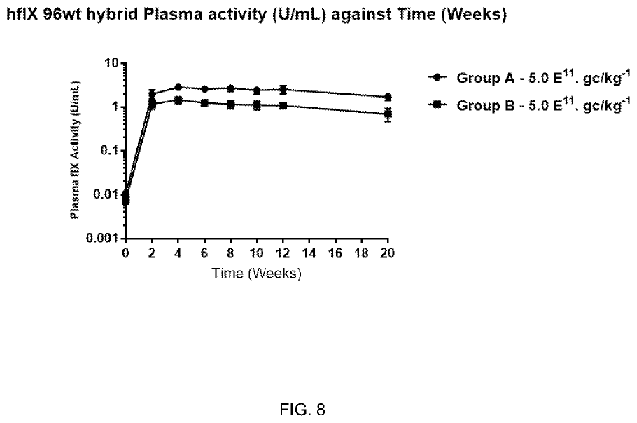

Turning to FIGS. 8 and 9, Additionally, in vivo studies were performed in hemophilia B mouse model using AAV2/8 vectors comparing AAV2-fIX-V86A-PRO2 (Group A) and AAV2-fIX-V86A-PRO2B (Group B). Data supports stable plasma fIX expression at therapeutics levels.

FIX Alpah, Beta, Delta and fIX Gamma (+Padua for each of Gamma and Beta) were liver codon optimized to optimize liver codon adaptation indec and minimize mRNA free energy. Four cDNA sequences were selected for each.

Liver Codon Optimized Sequences for fIX optimized sequence Alpha (no Padua), SEQ ID NO: 52 are SEQ ID NOS: 28-31.

hfIX Optimized Sequence Alpha 1
(SEQ ID NO: 28)
ATGCAGAGGGTGAACATGATCATGGCTGAGTCCCCTGGCC
TCATCACCATCTGCCTCCTGGGCTACCTCCTGAGTGCTGA
GTGCACAGTGTTCCTGGACCATGAGAATGCCAACAAGATC
CTCAACAGGCCCAAGAGGTACAACTCTGGCAAGCTGGAGG
AGTTTGTGCAGGGCAACCTGGAGAGGGAGTGCATGGAGGA
GAAGTGCTCCTTTGAGGAGGCCAGGGAGGTCTTTGAGAAC
ACAGAGAGGACCACAGAGTTCTGGAAGCAGTATGTGGATG
GGGACCAGTGTGAGTCCAACCCCTGCCTCAATGGGGGCTC
CTGCAAGGATGACATCAACTCCTATGAGTGCTGGTGCCCC
TTTGGCTTTGAGGGCAAGAACTGTGAGCTGGATGCCACCT
GCAACATCAAGAATGGGAGGTGTGAGCAGTTCTGCAAGAA
CTCTGCTGACAACAAGGTGGTGTGCTCCTGCACTGAGGGC
TACAGGCTGGCTGAGAACCAGAAGTCCTGTGAGCCTGCTG
TGCCCTTCCCCTGTGGGAGGGTGAGTGTGTCCCAGACCTC
CAAGCTCACCAGGGCTGAGACTGTGTTCCCTGATGTGGAC
TATGTGAACTCCACTGAGGCTGAGACCATCCTGGACAACA
TCACCCAGAGCACCCAGAGCTTCAATGACTTCACCAGGGT
GGTGGGGGGGGAGGATGCCAAGCCTGGCCAGTTCCCCTGG
CAGGTGGTGCTCAATGGCAAGGTGGATGCCTTCTGTGGGG
GCTCCATTGTGAATGAGAAGTGGATTGTGACTGCTGCCCA
CTGTGTGGAGACAGGGGTGAAGATCACAGTGGTGGCTGGG
GAGCACAACATTGAGGAGACAGAGCACACAGAGCAGAAGA
GGAATGTGATCAGGATCATCCCCCACCACAACTACAATGC
TGCCATCAACAAGTACAACCATGACATTGCCCTCCTGGAG
CTGGATGAGCCCCTGGTGCTCAACTCCTATGTGACCCCCA
TCTGCATTGCCAACAAGGAGTACACCAACATCTTCCTCAA
GTTTGGCTCTGGCTATGTGAGTGGCTGGGGGAGGGTGTTC
CACAAGGGGAGGAGTGCCTCTGTGCTCCAGTACCTGAGGG
TGCCCCTGGTGGACAGGGCCACCTGCCTGAGGAGCACCAA
GTTCACCATCTACAACAACATGTTCTGTGCTGGCTTCCAT
GAGGGGGGGAGGGACTCCTGCCAGGGGGACTCTGGGGGCC
CCCATGTGACTGAGGTGGAGGGCACCTCCTTCCTCACTGG
CATCATCTCCTGGGGGGAGGAGTGTGCCATGAAGGGCAAG
TATGGCATCTACACCAAGGTGAGCAGGTATGTGAACTGGA
TCAAGGAGAAGACCAAGTTGACCTGA
hfIX Optimized Sequence Alpha 2
(SEQ ID NO: 29)
ATGCAGAGGGTGAACATGATCATGGCTGAGTCCCCTGGCC
TGATCACCATCTGCCTCCTGGGCTACCTGCTGAGTGCTGA
GTGCACAGTGTTCCTGGACCATGAGAATGCCAACAAGATC
CTGAACAGGCCCAAGAGGTACAACTCTGGGAAGCTGGAGG
AGTTTGTCCAGGGGAACCTGGAGAGGGAGTGCATGGAGGA
GAAGTGCAGCTTTGAGGAGGCCAGGGAGGTGTTTGAGAAC
ACAGAGAGGACCACAGAGTTCTGGAAGCAGTATGTGGATG
GAGACCAGTGTGAGTCCAACCCCTGCCTGAATGGGGGCAG
CTGCAAGGATGACATCAACAGCTATGAGTGCTGGTGCCCC
TTTGGCTTTGAGGGGAAGAACTGTGAGCTGGATGCCACCT
GCAACATCAAGAATGGCAGGTGTGAGCAGTTCTGCAAGAA
CTCTGCTGATAACAAGGTGGTGTGCAGCTGCACAGAGGGC
TATAGGCTGGCTGAGAACCAGAAGAGCTGTGAGCCTGCTG
TGCCCTTCCCCTGTGGCAGGGTGAGTGTGTCCCAGACCTC
CAAGCTGACCAGGGCTGAGACAGTCTTCCCTGATGTGGAC
TATGTCAACAGCACAGAGGCTGAGACCATCCTGGACAACA
TCACCCAGTCCACCCAGAGCTTCAATGACTTCACCAGGGT
GGTGGGGGGGGAGGATGCCAAGCCTGGGCAGTTCCCCTGG
CAGGTGGTGCTGAATGGCAAGGTGGATGCCTTCTGTGGGG
GGAGCATTGTCAATGAGAAGTGGATTGTCACAGCTGCCCA
CTGTGTGGAGACAGGGGTGAAGATCACAGTGGTGGCTGGA
GAGCACAACATTGAGGAGACAGAGCACACAGAGCAGAAGA
GGAATGTCATCAGGATCATCCCCCACCACAACTACAATGC
TGCCATCAACAAGTACAACCATGACATTGCCCTGCTGGAG
CTGGATGAGCCCCTGGTGCTGAACAGCTATGTCACCCCCA
TCTGCATTGCCAACAAGGAGTACACCAACATCTTCCTGAA
GTTTGGCTCTGGCTATGTCTCTGGCTGGGGCAGGGTGTTC
CACAAGGGCAGATCTGCCTCTGTCCTGCAGTACCTGAGGG
TCCCCCTGGTGGATAGGGCCACCTGCCTGAGGAGCACCAA
GTTCACCATCTACAACAACATGTTCTGTGCTGGGTTCCAT
GAGGGGGGGAGGGACTCCTGCCAGGGGGATAGTGGAGGGC
CCCATGTGACAGAGGTGGAGGGCACCTCCTTCCTCACAGG
CATCATCTCCTGGGGGGAGGAGTGTGCCATGAAGGGCAAG
TATGGCATCTACACCAAGGTCAGCAGGTATGTCAACTGGA
TCAAGGAGAAGACCAAGCTCACCTGA
hfIX Optimized Sequence Alpha 3
(SEQ ID NO: 30)
ATGCAGAGGGTCAACATGATCATGGCTGAGAGCCCTGGGC
TCATCACCATCTGCCTGCTGGGCTACCTGCTGTCTGCTGA
GTGCACTGTGTTCCTGGACCATGAGAATGCCAACAAGATC
CTGAACAGACCCAAGAGGTACAACTCAGGCAAGCTGGAGG
AGTTTGTGCAGGGCAACCTGGAGAGGGAGTGCATGGAGGA
GAAGTGCTCCTTTGAGGAGGCCAGAGAGGTCTTTGAGAAC
ACAGAGAGGACCACTGAGTTCTGGAAGCAGTATGTGGATG
GGGACCAGTGTGAGTCCAACCCCTGCCTGAATGGAGGCAG
CTGCAAGGATGACATCAACAGCTATGAGTGCTGGTGCCCC
TTTGGCTTTGAGGGCAAGAACTGTGAGCTGGATGCCACCT
GCAACATCAAGAATGGCAGATGTGAGCAGTTCTGCAAGAA
CTCAGCTGACAACAAGGTGGTGTGCAGCTGCACAGAGGGC
TACAGACTGGCTGAGAACCAGAAGAGCTGTGAGCCTGCTG
TGCCCTTCCCCTGTGGCAGGGTCTCTGTCAGCCAGACCTC
CAAGCTCACCAGAGCTGAGACTGTGTTCCCTGATGTGGAC
TATGTGAACAGCACAGAGGCTGAGACCATCCTGGACAACA
TCACCCAGAGCACCCAGTCCTTCAATGACTTCACCAGAGT
GGTGGGGGGGGAGGATGCCAAGCCAGGGCAGTTCCCCTGG
CAGGTGGTGCTGAATGGCAAGGTGGATGCCTTCTGTGGGG
GCTCCATTGTGAATGAGAAGTGGATTGTGACAGCTGCCCA
CTGTGTGGAGACAGGGGTCAAGATCACAGTGGTGGCTGGA
GAGCACAACATAGAGGAGACAGAGCACACAGAGCAGAAGA
GGAATGTCATCAGGATCATCCCCCACCACAACTACAATGC
AGCCATCAACAAGTACAACCATGACATAGCCCTGCTGGAG
CTGGATGAGCCCCTGGTGCTGAACAGCTATGTGACCCCCA
TCTGCATAGCCAACAAGGAGTACACCAACATCTTCCTGAA
GTTTGGCTCTGGCTATGTGTCTGGCTGGGGCAGGGTCTTC
CACAAGGGCAGATCTGCCAGTGTGCTGCAGTACCTGAGAG
TGCCCCTGGTGGACAGGGCCACCTGCCTGAGGAGCACTAA
GTTCACCATCTACAACAACATGTTCTGTGCTGGCTTCCAT
GAGGGGGGCAGAGACAGCTGCCAGGGGGACTCTGGGGGCC
CCCATGTGACAGAGGTGGAGGGCACCAGCTTCCTGACAGG
CATCATCAGCTGGGGGGAGGAGTGTGCCATGAAGGGCAAG
TATGGCATCTACACCAAGGTCAGCAGATATGTGAACTGGA
TCAAGGAGAAGACCAAGCTGACCTGA
hfIX Optimized Sequence Alpha 4
(SEQ ID NO: 31)
ATGCAGAGGGTGAACATGATCATGGCTGAGTCCCCTGGCC
TGATCACCATCTGCCTGCTGGGCTACCTGCTGTCTGCTGA
GTGCACAGTGTTCCTGGACCATGAGAATGCCAACAAGATC
CTGAACAGGCCCAAGAGGTACAACAGTGGCAAGCTGGAGG
AGTTTGTGCAGGGCAACCTGGAGAGGGAGTGCATGGAGGA
GAAGTGCAGCTTTGAGGAGGCCAGGGAGGTGTTTGAGAAC
ACAGAGAGGACCACAGAGTTCTGGAAGCAGTATGTGGATG
GGGACCAGTGTGAGTCCAACCCCTGCCTGAATGGGGGCAG
CTGCAAGGATGACATCAACTCCTATGAGTGCTGGTGCCCC
TTTGGCTTTGAGGGCAAGAACTGTGAGCTGGATGCCACCT
GCAACATCAAGAATGGCAGGTGTGAGCAGTTCTGCAAGAA
CAGTGCTGACAACAAGGTGGTGTGCTCCTGCACAGAGGGC
TACAGGCTGGCTGAGAACCAGAAGTCCTGTGAGCCTGCTG
TGCCCTTCCCCTGTGGCAGGGTGTCTGTGTCCCAGACCTC
CAAGCTGACCAGGGCTGAGACAGTGTTCCCTGATGTGGAC
TATGTGAACTCCACAGAGGCTGAGACCATCCTGGACAACA
TCACCCAGTCCACCCAGAGCTTCAATGACTTCACCAGGGT
GGTGGGGGGGGAGGATGCCAAGCCTGGCCAGTTCCCCTGG
CAGGTGGTGCTGAATGGCAAGGTGGATGCCTTCTGTGGGG
GCTCCATAGTGAATGAGAAGTGGATTGTGACTGCTGCCCA
CTGTGTGGAGACAGGGGTGAAGATCACAGTGGTGGCTGGG
GAGCACAACATTGAGGAGACAGAGCACACAGAGCAGAAGA
GGAATGTGATCAGGATCATCCCCCACCACAACTACAATGC
TGCCATCAACAAGTACAACCATGACATTGCCCTGCTGGAG
CTGGATGAGCCCCTGGTGCTGAACTCCTATGTGACCCCCA
TCTGCATAGCCAACAAGGAGTACACCAACATCTTCCTGAA
GTTTGGCAGTGGCTATGTGTCTGGCTGGGGCAGGGTGTTC
CACAAGGGCAGGTCTGCCTCTGTGCTGCAGTACCTGAGGG
TGCCCCTGGTGGACAGGGCCACCTGCCTGAGGAGCACCAA
GTTCACCATCTACAACAACATGTTCTGTGCTGGCTTCCAT
GAGGGGGGCAGGGACTCCTGCCAGGGGGACTCTGGGGGCC
CCCATGTGACTGAGGTGGAGGGCACCTCCTTCCTGACAGG
CATCATCTCCTGGGGGGAGGAGTGTGCCATGAAGGGCAAG
TATGGCATCTACACCAAGGTGAGCAGGTATGTGAACTGGA
TCAAGGAGAAGACCAAGCTGACCTGA
Liver Codon Optimized Sequences for fIX
optimized sequence Beta (no Padua),
SEQ ID NO: 53 are SEQ ID NOS: 32-35.
hfIX Optimized Sequence Beta 1
(SEQ ID NO: 32)
ATGCAGAGGGTGAACATGATCATGGCTGAGAGCCCTGGGC
TGATCACCATCTGCCTGCTGGGGTACCTGCTGAGTGCTGA
GTGCACTGTGTTCCTGGACCATGAGAATGCCAACAAGATC
CTCAACAGGCCCAAGAGGTACAACAGTGGCAAGCTGGAGG
AGTTTGTGCAGGGCAACCTGGAGAGGGAGTGCATGGAGGA
GAAGTGCAGCTTTGAGGAGGCCAGGGAGGTGTTTGAGAAC
ACAGAGAGGACCACAGAGTTCTGGAAGCAGTATGTGGATG
GGGACCAGTGTGAGTCCAACCCCTGCCTGAATGGGGGCAG
CTGCAAGGATGACATCAACAGCTATGAGTGCTGGTGCCCC
TTTGGCTTTGAGGGCAAGAACTGTGAGCTGGATGCCACCT
GCAACATCAAGAATGGCAGGTGTGAGCAGTTCTGCAAGAA
CAGTGCTGACAACAAGGTGGTGTGCAGCTGCACTGAGGGC
TACAGGCTGGCTGAGAACCAGAAGTCCTGTGAGCCTGCAG
TGCCCTTCCCCTGTGGCAGGGTGTCTGTGTCCCAGACCAG
CAAGCTCACCAGGGCTGAGACTGTGTTCCCTGATGTGGAC
TATGTCAACAGCACAGAGGCTGAGACCATCCTGGACAACA
TCACCCAGAGCACCCAGAGCTTCAATGACTTCACCAGGGT
GGTGGGGGGGGAGGATGCCAAGCCAGGGCAGTTCCCCTGG
CAGGTGGTGCTGAATGGCAAGGTGGATGCCTTCTGTGGGG
GCAGCATTGTCAATGAGAAGTGGATTGTCACAGCAGCCCA
CTGTGTGGAGACTGGGGTGAAGATCACAGTGGTGGCTGGG
GAGCACAACATTGAGGAGACAGAGCACACAGAGCAGAAGA
GGAATGTCATCAGGATCATCCCCCACCACAACTACAATGC
TGCCATCAACAAGTACAACCATGACATTGCCCTGCTGGAG
CTGGATGAGCCCCTGGTGCTGAACAGCTATGTCACCCCCA
TCTGCATTGCCAACAAGGAGTACACCAACATCTTCCTGAA
GTTTGGCAGTGGCTATGTCAGTGGCTGGGGCAGGGTGTTC
CACAGGGGCAGGTCTGCCAGTGTGCTGCAGTACCTGAGGG
TGCCCCTGGTGGACAGGGCCACCTGCCTGAGGTCCACCAA
GTTCACCATCTACAACAACATGTTCTGTGCTGGCTTCCAT
GAGGGGGGCAGGGACAGCTGCCAGGGGGACTCTGGGGGCC
CCCATGTCACTGAGGTGGAGGGCACCAGCTTCCTGACAGG
CATCATCAGCTGGGGGGAGGAGTGTGCCATGAAGGGCAAG
TATGGCATCTACACCAAGGTCAGCAGGTATGTGAACTGGA
TCAAGGAGAAGACCAAGCTCACCTGA
hfIX Optimized Sequence Beta 2
(SEQ ID NO: 33)
ATGCAGAGGGTGAACATGATCATGGCTGAGTCCCCTGGCC
TGATCACCATCTGCCTGCTGGGCTACCTGCTGTCTGCTGA
GTGCACAGTGTTCCTGGACCATGAGAATGCCAACAAGATC
CTGAACAGGCCCAAGAGGTACAACTCTGGCAAGCTGGAGG
AGTTTGTGCAGGGCAACCTGGAGAGGGAGTGCATGGAGGA
GAAGTGCTCCTTTGAGGAGGCCAGGGAGGTGTTTGAGAAC
ACAGAGAGGACCACAGAGTTCTGGAAGCAGTATGTGGATG
GAGACCAGTGTGAGTCCAACCCTTGCCTGAATGGAGGCTC
CTGCAAGGATGACATCAACTCCTATGAGTGCTGGTGCCCC
TTTGGCTTTGAGGGCAAGAACTGTGAGCTGGATGCCACCT
GCAACATCAAGAATGGCAGGTGTGAGCAGTTCTGCAAGAA
CTCTGCTGACAACAAGGTGGTGTGCTCCTGCACAGAGGGC
TACAGGCTGGCTGAGAACCAGAAGTCCTGTGAGCCAGCTG
TGCCCTTCCCTTGTGGCAGGGTGTCTGTGTCCCAGACCTC
CAAGCTGACCAGGGCTGAGACAGTGTTCCCTGATGTGGAC
TATGTGAACTCCACAGAGGCTGAGACCATCCTGGACAACA
TCACCCAGTCCACCCAGTCCTTCAATGACTTCACCAGGGT
GGTGGGAGGTGAGGATGCCAAGCCTGGCCAGTTCCCTTGG
CAGGTGGTGCTGAATGGCAAGGTGGATGCCTTCTGTGGAG
GCTCCATTGTGAATGAGAAGTGGATTGTGACAGCTGCCCA
CTGTGTGGAGACAGGAGTGAAGATCACAGTGGTGGCTGGT
GAGCACAACATTGAGGAGACAGAGCACACAGAGCAGAAGA
GGAATGTGATCAGGATCATCCCCCACCACAACTACAATGC
TGCCATCAACAAGTACAACCATGACATTGCCCTGCTGGAG
CTGGATGAGCCCCTGGTGCTGAACTCCTATGTGACCCCCA
TCTGCATTGCCAACAAGGAGTACACCAACATCTTCCTGAA
GTTTGGCTCTGGCTATGTGTCAGGATGGGGCAGGGTGTTC
CACAGGGGCAGGTCTGCCTCTGTGCTGCAGTACCTGAGGG
TGCCCCTGGTGGACAGGGCCACCTGCCTGAGGTCCACCAA
GTTCACCATCTACAACAACATGTTCTGTGCTGGCTTCCAT
GAGGGAGGCAGGGACTCCTGCCAGGGTGACTCAGGAGGCC
CCCATGTGACAGAGGTGGAGGGCACCTCCTTCCTGACAGG
CATCATCAGCTGGGGAGAGGAGTGTGCCATGAAGGGCAAG
TATGGCATCTACACCAAGGTGTCCAGGTATGTGAACTGGA
TCAAGGAGAAGACCAAGCTGACCTGA
hfIX Optimized Sequence Beta 3
(SEQ ID NO: 34)
ATGCAGAGAGTCAACATGATCATGGCAGAGTCACCTGGAC
TGATCACCATCTGCCTGCTGGGCTACCTGCTCAGTGCAGA
GTGCACAGTCTTCCTGGACCATGAGAATGCCAACAAGATC
CTGAACAGACCCAAGAGGTACAACTCTGGCAAGCTGGAGG
AGTTTGTGCAGGGCAACCTGGAGAGAGAGTGCATGGAGGA
GAAGTGCTCCTTTGAGGAGGCCAGAGAGGTCTTTGAGAAC
ACTGAGAGGACCACTGAGTTCTGGAAGCAGTATGTGGATG
GTGATCAGTGTGAGTCCAACCCATGCCTGAATGGAGGCTC
CTGCAAGGATGACATCAACTCCTATGAGTGCTGGTGCCCA
TTTGGCTTTGAGGGCAAGAACTGTGAGCTGGATGCAACAT
GCAACATCAAGAATGGCAGATGTGAGCAGTTCTGCAAGAA
CAGTGCAGACAACAAGGTGGTGTGCTCCTGCACAGAGGGC
TACAGACTGGCTGAGAACCAGAAGTCATGTGAGCCTGCTG
TGCCCTTCCCCTGTGGCAGAGTGAGTGTGTCCCAGACCAG
CAAGCTGACCAGAGCTGAGACAGTCTTCCCTGATGTGGAC
TATGTGAACTCCACAGAGGCAGAGACCATCCTGGACAACA
TCACACAGTCCACACAGTCCTTCAATGACTTCACCAGAGT
GGTTGGTGGTGAGGATGCCAAGCCTGGACAGTTCCCCTGG
CAGGTGGTGCTCAATGGCAAGGTGGATGCCTTCTGTGGAG
GCTCCATTGTCAATGAGAAGTGGATTGTCACAGCTGCCCA
CTGTGTGGAGACTGGTGTGAAGATCACTGTGGTGGCTGGT
GAGCACAACATTGAGGAGACAGAGCACACTGAGCAGAAGA
GGAATGTCATCAGGATCATCCCACACCACAACTACAATGC
AGCCATCAACAAGTACAACCATGACATTGCCCTGCTGGAG
CTGGATGAGCCCCTGGTGCTGAACAGCTATGTGACCCCCA
TCTGCATAGCCAACAAGGAGTACACCAACATCTTCCTGAA
GTTTGGCTCTGGCTATGTGTCTGGCTGGGGCAGAGTCTTC
CACAGAGGCAGATCTGCCTCTGTGCTGCAGTACCTGAGAG
TGCCTCTGGTGGACAGAGCAACCTGCCTGAGAAGCACCAA
GTTCACCATCTACAACAACATGTTCTGTGCAGGCTTCCAT
GAGGGAGGCAGAGACTCCTGCCAGGGAGACTCTGGTGGGC
CACATGTGACAGAGGTGGAGGGCACATCCTTCCTGACAGG
CATCATCTCCTGGGGAGAGGAGTGTGCCATGAAGGGCAAG
TATGGCATCTACACCAAGGTGTCCAGGTATGTCAACTGGA
TCAAGGAGAAGACCAAGCTGACCTGA
hfIX Optimized Sequence Beta 4
(SEQ ID NO: 35)
ATGCAGAGAGTGAACATGATCATGGCTGAGTCCCCTGGCC
TGATCACCATCTGCCTGCTGGGCTACCTGCTGTCTGCTGA
GTGCACAGTGTTCCTGGATCATGAGAATGCCAACAAGATC
CTGAACAGACCCAAGAGATACAACTCTGGCAAGCTGGAGG
AGTTTGTGCAGGGCAACCTGGAGAGAGAGTGCATGGAGGA
GAAGTGCTCCTTTGAGGAGGCCAGAGAGGTGTTTGAGAAC
ACAGAGAGAACCACAGAGTTCTGGAAGCAGTATGTGGATG
GAGATCAGTGTGAGTCCAACCCTTGCCTGAATGGAGGCTC
CTGCAAGGATGATATCAACTCCTATGAGTGCTGGTGCCCA
TTTGGCTTTGAGGGCAAGAATTGTGAGCTGGATGCCACCT
GCAACATCAAGAATGGCAGATGTGAGCAGTTCTGCAAGAA
CTCTGCTGATAACAAGGTGGTGTGCTCCTGCACAGAGGGC
TACAGACTGGCTGAGAACCAGAAGTCCTGTGAGCCTGCTG
TGCCTTTCCCTTGTGGCAGAGTGTCTGTGTCCCAGACCTC
CAAGCTGACCAGAGCTGAGACAGTGTTCCCTGATGTGGAT
TATGTGAACTCCACAGAGGCTGAGACCATCCTGGATAACA
TCACCCAGTCCACCCAGTCCTTCAATGATTTCACCAGAGT
GGTGGGAGGAGAGGATGCCAAGCCTGGCCAGTTCCCCTGG
CAGGTGGTGCTGAATGGCAAGGTGGATGCCTTCTGTGGAG
GCTCCATTGTGAATGAGAAGTGGATTGTGACAGCTGCCCA
CTGTGTGGAGACAGGAGTGAAGATCACAGTGGTGGCTGGA
GAGCACAACATTGAGGAGACAGAGCACACAGAGCAGAAGA
GAAATGTGATCAGAATCATCCCTCACCACAACTACAATGC
TGCCATCAACAAGTACAACCATGATATTGCCCTGCTGGAG
CTGGATGAGCCCCTGGTGCTGAACTCCTATGTGACCCCTA
TCTGCATTGCCAACAAGGAGTACACCAACATCTTCCTGAA
GTTTGGCTCTGGCTATGTGTCTGGCTGGGGCAGAGTGTTC
CACAGAGGCAGATCTGCCTCTGTGCTGCAGTACCTGAGAG
TGCCTCTGGTGGATAGAGCCACCTGCCTGAGATCCACCAA
GTTCACCATCTACAACAACATGTTCTGTGCTGGCTTCCAT
GAGGGAGGCAGAGATTCCTGCCAGGGAGATTCTGGAGGCC
CTCATGTGACAGAGGTGGAGGGCACCTCCTTCCTGACAGG
CATCATCTCCTGGGGAGAGGAGTGTGCCATGAAGGGCAAG
TATGGCATCTACACCAAGGTGTCCAGATATGTGAACTGGA
TCAAGGAGAAGACCAAGCTGACCTGA

Liver Codon Optimized Sequences for fIX optimized sequence Delta (no Padua), SEQ ID NO: 54 are SEQ ID NOS: 36-39.

hFIX Optimized Sequence Delta 1
(SEQ ID NO: 36)
ATGCAGAGGGTGAACATGATCATGGCAGAGAGCCCTGGCC
TGATCACCATCTGCCTGCTGGGCTACCTGCTGTCAGCAGA
GTGCACAGTGTTCCTGGACCATGAGAATGCCAACAAGATC
CTGAACAGGCCCAAGAGGTACAACAGTGGCAAGCTGGAGG
AGTTTGTCCAGGGCAACCTGGAGAGGGAGTGCATGGAGGA
GAAGTGCTCCTTTGAGGAGGCCAGGGAGGTGTTTGAGAAC
ACAGAGAGGACAACAGAGTTCTGGAAGCAGTATGTTGATG
GTGACCAGTGTGAGAGCAACCCCTGCCTGAATGGTGGCTC
CTGCAAGGATGACATCAACAGCTATGAGTGCTGGTGCCCC
TTTGGCTTTGAGGGCAAGAACTGTGAGCTGGATGCCACCT
GCAACATCAAGAATGGCAGGTGTGAGCAGTTCTGCAAGAA
CAGTGCTGACAACAAGGTGGTGTGCTCCTGCACAGAGGGC
TACAGGCTGGCAGAGAACCAGAAGAGCTGTGAGCCTGCTG
TCCCCTTCCCCTGTGGCAGGGTGTCAGTGTCCCAGACCAG
CAAGCTGACCAGGGCAGAGACAGTGTTCCCTGATGTTGAC
TATGTCAACAGCACAGAGGCAGAGACCATCCTGGACAACA
TCACCCAGAGCACCCAGTCCTTCAATGACTTCACCAGGGT
GGTGGGGGGGGAGGATGCCAAGCCTGGCCAGTTCCCCTGG
CAGGTGGTGCTGAATGGCAAGGTGGATGCCTTCTGTGGTG
GCAGCATTGTCAATGAGAAGTGGATAGTGACAGCTGCCCA
CTGTGTGGAGACAGGGGTGAAGATAACAGTGGTGGCAGGG
GAGCACAACATTGAGGAGACAGAGCACACAGAGCAGAAGA
GGAATGTCATCAGGATCATCCCCCACCACAACTACAATGC
TGCCATCAACAAGTACAACCATGACATTGCCCTGCTGGAG
CTGGACAAGCCCCTGACCCTGAACAGCTATGTCACCCCCA
TCTGCATTGCCAACAGGGAGTACACCAACATCTTCCTGAA
GTTTGGCAGTGGCTATGTCTCTGGCTGGGGCAGGGTGTTC
CACAGGGGCAGGAGTGCCAGTGTCCTGCAGTACCTGAGGG
TGCCCCTGGTGGACAGGGCCACCTGCCTGAGGAGCACCAA
GTTCACCATCTACAACAACATGTTCTGTGCTGGCTTCCAT
GAGGGGGGCAGGGACAGCTGCCAGGGGGACAGTGGTGGCC
CCCATGTGACAGAGGTGGAGGGCACCTCCTTCCTGACAGG
CATCATCTCCTGGGGGGAGGAGTGTGCCATGAAGGGCAAG
TATGGCATCTACACCAAGGTGAGCAGGTATGTCAACTGGA
TCAAGGAGAAGACCAAGCTGACATGA
FIX Optimized Sequence Delta 2
(SEQ ID NO: 37)
ATGCAGAGGGTCAACATGATCATGGCTGAGTCACCTGGCC
TGATCACCATCTGCCTGCTGGGCTACCTGCTGTCTGCTGA
GTGCACAGTGTTCCTGGACCATGAGAATGCCAACAAGATC
CTGAACAGGCCCAAGAGGTACAACAGTGGCAAGCTGGAGG
AGTTTGTGCAGGGCAACCTGGAGAGGGAGTGCATGGAGGA
GAAGTGCAGCTTTGAGGAGGCCAGGGAGGTCTTTGAGAAC
ACAGAGAGGACCACAGAGTTCTGGAAGCAGTATGTGGATG
GTGACCAGTGTGAGAGCAACCCCTGCCTGAATGGTGGCTC
CTGCAAGGATGACATCAACAGCTATGAGTGCTGGTGCCCC
TTTGGCTTTGAGGGCAAGAACTGTGAGCTGGATGCCACCT
GCAACATCAAGAATGGCAGGTGTGAGCAGTTCTGCAAGAA
CAGTGCTGACAACAAGGTGGTGTGCAGCTGCACAGAGGGC
TACAGGCTGGCTGAGAACCAGAAGTCCTGTGAGCCTGCTG
TGCCATTCCCCTGTGGCAGGGTCAGTGTCAGCCAGACCAG
CAAGCTGACCAGGGCTGAGACAGTGTTCCCTGATGTGGAC
TATGTGAACAGCACAGAGGCTGAGACCATCCTGGACAACA
TCACCCAGAGCACCCAGAGCTTCAATGACTTCACCAGAGT
GGTGGGTGGTGAGGATGCCAAGCCTGGCCAGTTCCCCTGG
CAGGTGGTGCTCAATGGCAAGGTGGATGCCTTCTGTGGTG
GCAGCATTGTCAATGAGAAGTGGATTGTCACAGCTGCACA
CTGTGTGGAGACAGGTGTCAAGATCACAGTGGTGGCTGGT
GAGCACAACATTGAGGAGACAGAGCACACAGAGCAGAAGA
GGAATGTGATCAGGATCATCCCACACCACAACTACAATGC
TGCCATCAACAAGTACAACCATGACATTGCACTGCTGGAG
CTGGACAAGCCACTGACCCTGAACAGCTATGTGACCCCCA
TCTGCATTGCCAACAGGGAGTACACCAACATCTTCCTGAA
GTTTGGCTCTGGCTATGTCAGTGGCTGGGGCAGGGTGTTC
CACAGAGGCAGGTCTGCCAGTGTGCTGCAGTACCTGAGGG
TGCCACTGGTGGACAGGGCCACCTGCCTCAGGTCCACCAA
GTTCACCATCTACAACAACATGTTCTGTGCTGGCTTCCAT
GAGGGTGGCAGGGACAGCTGCCAGGGTGACTCTGGTGGCC
CACATGTGACAGAGGTGGAGGGCACCAGCTTCCTGACAGG
CATCATCAGCTGGGGTGAGGAGTGTGCCATGAAGGGCAAG
TATGGCATCTACACCAAGGTCAGCAGGTATGTCAACTGGA
TCAAGGAGAAGACCAAGCTGACCTGA
hfIX Optimized Sequence Delta 3
(SEQ ID NO: 38)
ATGCAGAGGGTCAACATGATCATGGCTGAGTCACCAGGCT
TGATCACCATCTGCCTGCTGGGCTACCTGCTGTCAGCTGA
GTGCACAGTGTTTCTTGACCATGAGAATGCCAACAAGATC
CTCAACAGGCCCAAGAGGTACAACAGTGGCAAGCTGGAGG
AGTTTGTCCAAGGCAACCTGGAGAGAGAGTGCATGGAGGA
GAAGTGCAGCTTTGAGGAGGCCAGAGAGGTGTTTGAGAAC
ACTGAGAGGACCACTGAGTTCTGGAAGCAGTATGTGGATG
GTGACCAGTGTGAGTCCAACCCCTGTCTCAATGGGGGCAG
CTGCAAGGATGACATCAACAGCTATGAGTGCTGGTGCCCC
TTTGGCTTTGAGGGCAAGAACTGTGAGCTTGATGCCACCT
GCAACATCAAGAATGGCAGGTGTGAGCAGTTCTGCAAGAA
CTCAGCTGACAACAAGGTGGTGTGCAGCTGCACTGAGGGC
TACAGGCTGGCTGAGAACCAGAAGAGCTGTGAGCCAGCTG
TGCCCTTCCCCTGTGGCAGGGTGAGTGTGAGCCAGACCAG
CAAGCTGACCAGGGCTGAGACAGTGTTCCCTGATGTTGAC
TATGTCAACAGCACTGAGGCTGAGACCATCTTGGACAACA
TCACTCAGTCAACCCAGAGCTTCAATGACTTCACCAGGGT
GGTGGGGGGTGAGGATGCTAAGCCAGGCCAGTTCCCCTGG
CAGGTGGTGCTCAATGGCAAGGTTGATGCCTTCTGTGGTG
GCTCAATTGTCAATGAGAAGTGGATTGTGACAGCTGCCCA
CTGTGTTGAGACTGGGGTCAAGATCACAGTGGTGGCTGGT
GAGCACAACATTGAGGAGACTGAGCACACTGAGCAGAAGA
GGAATGTGATCAGGATCATCCCTCATCACAACTACAATGC
TGCCATCAACAAGTACAATCATGACATAGCCCTGCTTGAG
CTTGACAAGCCCCTGACCCTCAACAGCTATGTCACCCCCA
TCTGCATTGCCAACAGGGAGTACACCAACATCTTCCTCAA
GTTTGGCTCAGGCTATGTCTCAGGCTGGGGCAGGGTGTTC
CACAGGGGCAGGTCAGCCTCAGTGCTGCAGTACCTCAGGG
TGCCACTGGTGGACAGGGCCACCTGCCTGAGGTCAACCAA
GTTCACCATCTACAACAACATGTTCTGTGCTGGCTTCCAT
GAGGGGGGCAGAGACAGCTGCCAGGGTGACTCAGGTGGGC
CCCATGTGACTGAGGTTGAGGGCACCAGCTTCCTCACTGG
CATCATCAGCTGGGGTGAGGAGTGTGCCATGAAGGGCAAG
TATGGCATCTACACCAAGGTGAGCAGGTATGTCAACTGGA
TCAAGGAGAAGACCAAGCTGACCTGA
FIX Optimized Sequence Delta 4
(SEQ ID NO: 39)
ATGCAGAGAGTCAACATGATCATGGCTGAGTCCCCTGGGC
TGATCACCATCTGCCTGCTGGGCTACCTGCTGTCTGCTGA
GTGCACAGTCTTCCTGGACCATGAGAATGCCAACAAGATC
CTCAACAGGCCCAAGAGGTACAACTCAGGCAAGCTGGAGG
AGTTTGTCCAGGGCAACCTGGAGAGAGAGTGCATGGAGGA
GAAGTGCTCCTTTGAGGAGGCCAGAGAGGTCTTTGAGAAC
ACAGAGAGAACCACAGAGTTCTGGAAGCAGTATGTGGATG
GTGACCAGTGTGAGAGCAACCCCTGCCTGAATGGAGGCTC
CTGCAAGGATGACATCAACAGCTATGAGTGCTGGTGCCCC
TTTGGCTTTGAGGGCAAGAACTGTGAGCTGGATGCCACCT
GCAACATCAAGAATGGCAGATGTGAGCAGTTCTGCAAGAA
CTCTGCAGACAACAAGGTGGTCTGCAGCTGCACAGAGGGC
TACAGGCTGGCTGAGAACCAGAAGAGCTGTGAGCCTGCTG
TGCCCTTCCCCTGTGGCAGAGTGTCAGTCAGCCAGACCAG
CAAGCTGACCAGAGCAGAGACAGTCTTCCCTGATGTGGAC
TATGTGAACTCCACAGAGGCAGAGACCATCCTGGATAACA
TCACCCAGAGCACTCAGAGCTTCAATGATTTCACCAGGGT
GGTGGGTGGTGAGGATGCCAAGCCTGGCCAGTTCCCCTGG
CAGGTGGTCCTGAATGGCAAGGTGGATGCCTTCTGTGGAG
GCTCCATTGTGAATGAGAAGTGGATTGTCACTGCTGCTCA
CTGTGTGGAGACAGGAGTGAAGATCACTGTGGTGGCTGGA
GAGCACAACATAGAGGAGACAGAGCACACAGAGCAGAAGA
GAAATGTCATCAGGATCATCCCCCATCACAACTACAATGC
AGCCATCAACAAGTACAACCATGACATTGCCCTGCTGGAG
CTGGACAAGCCCCTCACCCTGAACTCCTATGTGACTCCCA
TCTGCATTGCCAACAGAGAGTACACCAACATCTTCCTGAA
GTTTGGCTCTGGCTATGTGTCTGGCTGGGGCAGAGTGTTC
CACAGGGGCAGGTCTGCCAGTGTGCTGCAGTACCTCAGAG
TGCCCCTGGTGGACAGAGCCACCTGCCTGAGGAGCACCAA
GTTCACCATCTACAACAACATGTTCTGTGCTGGCTTCCAT
GAGGGAGGCAGAGATAGCTGCCAGGGTGACTCTGGAGGCC
CCCATGTGACTGAGGTGGAGGGCACCTCCTTCCTGACAGG
CATCATCTCCTGGGGGGAGGAGTGTGCCATGAAGGGCAAG
TATGGCATCTACACCAAGGTGAGCAGGTATGTCAACTGGA
TCAAGGAGAAGACCAAGCTGACCTGA

Liver Codon Optimized Sequences for fIX optimized sequence Gamma (no Padua), SEQ ID NO: 55 are SEQ ID NOS: 40-43.

FIX Optimized Sequence Gamma 1
(SEQ ID NO: 40)
ATGCAGAGGGTCAACATGATCATGGCAGAGTCCCCTGGGC
TCATCACCATCTGCCTCCTGGGGTACCTGCTCAGTGCTGA
GTGCACAGTGTTCCTGGACCATGAGAATGCCAACAAGATC
CTGAACAGGCCCAAGAGGTACAACTCTGGGAAACTGGAGG
AGTTTGTCCAGGGGAACCTGGAGAGGGAGTGCATGGAGGA
GAAGTGCTCCTTTGAGGAGGCCAGGGAGGTCTTTGAGAAC
ACAGAGAGGACCACTGAGTTCTGGAAGCAGTATGTGGATG
GGGACCAGTGTGAGTCCAACCCCTGCCTCAATGGGGGCAG
CTGCAAGGATGACATCAACTCCTATGAGTGCTGGTGCCCC
TTTGGCTTTGAGGGCAAGAACTGTGAGCTGGATGCCACCT
GCAACATCAAGAATGGCAGATGTGAGCAGTTCTGCAAGAA
CTCTGCTGACAACAAGGTGGTGTGCAGCTGCACTGAGGGC
TACAGGCTGGCTGAGAACCAGAAGTCCTGTGAGCCAGCTG
TGCCCTTCCCCTGTGGCAGAGTCTCAGTGTCCCAGACCTC
CAAGCTGACCAGAGCTGAGACAGTGTTCCCAGATGTGGAC
TATGTGAACTCCACAGAGGCTGAGACCATCCTGGACAACA
TCACCCAGAGCACCCAGAGCTTCAATGACTTCACCAGGGT
GGTGGGAGGAGAAGATGCCAAGCCTGGGCAGTTCCCCTGG
CAGGTGGTGCTCAATGGCAAAGTGGATGCCTTCTGTGGAG
GCTCCATTGTCAATGAGAAGTGGATTGTCACAGCTGCCCA
CTGTGTGGAGACAGGGGTCAAGATCACAGTGGTGGCAGGG
GAGCACAACATTGAGGAGACAGAGCACACAGAGCAGAAGA
GGAATGTGATCAGGATCATCCCCCACCACAACTACAATGC
TGCCATCAACAAGTACAACCATGACATTGCCCTGCTGGAG
CTGGACAAGCCCCTGGTGCTGAACTCCTATGTGACCCCCA
TCTGCATTGCCAACAAGGAGTACACCAACATCTTCCTGAA
GTTTGGCTCTGGGTATGTCTCAGGCTGGGGCAGAGTGTTC
CACAGGGGCAGAAGTGCCTCAGTGCTGCAGTACCTCAGGG
TGCCCCTGGTGGACAGGGCCACTTGCCTCAGGAGCACCAA
GTTCACCATCTACAACAACATGTTCTGTGCAGGGTTCCAT
GAGGGGGGCAGGGACTCCTGCCAGGGGGACTCTGGGGGCC
CCCATGTCACAGAGGTGGAGGGCACCAGTTTCCTCACAGG
GATCATCTCCTGGGGTGAGGAGTGTGCCATGAAGGGCAAG
TATGGCATCTACACCAAGGTGTCCAGGTATGTGAACTGGA
TCAAGGAGAAGACCAAGCTGACCTGA
FIX Optimized Sequence Gamma 2
(SEQ ID NO: 41)
ATGCAGAGGGTCAACATGATCATGGCTGAGAGCCCTGGCC
TCATCACCATCTGCCTGCTGGGCTACCTGCTGTCAGCTGA
GTGCACAGTGTTCCTGGACCATGAGAATGCCAACAAGATC
CTCAACAGGCCCAAGAGGTACAACAGTGGCAAGCTGGAGG
AGTTTGTGCAGGGCAACCTGGAGAGGGAGTGCATGGAGGA
GAAGTGCAGCTTTGAGGAGGCCAGGGAGGTGTTTGAGAAC
ACAGAGAGGACAACAGAGTTCTGGAAGCAGTATGTTGATG
GTGACCAGTGTGAGAGCAACCCCTGCCTGAATGGGGGCAG
CTGCAAGGATGACATCAACAGCTATGAGTGCTGGTGCCCC
TTTGGCTTTGAGGGCAAGAACTGTGAGCTTGATGCCACCT
GCAACATCAAGAATGGCAGGTGTGAGCAGTTCTGCAAGAA
CAGTGCTGACAACAAGGTGGTGTGCAGCTGCACAGAGGGC
TACAGGCTGGCTGAGAACCAGAAGAGCTGTGAGCCTGCTG
TGCCCTTCCCCTGTGGCAGGGTGTCTGTGAGCCAGACAAG
CAAGCTGACCAGGGCTGAGACAGTGTTCCCTGATGTTGAC
TATGTCAACAGCACAGAGGCTGAGACCATCCTGGACAACA
TCACACAGTCAACACAGAGCTTCAATGACTTCACCAGGGT
GGTGGGGGGGGAGGATGCCAAGCCTGGCCAGTTCCCCTGG
CAGGTGGTGCTGAATGGCAAGGTTGATGCCTTCTGTGGGG
GCAGCATTGTCAATGAGAAGTGGATTGTGACAGCTGCCCA
CTGTGTTGAGACAGGGGTCAAGATCACAGTGGTGGCTGGT
GAGCACAACATTGAGGAGACAGAGCACACAGAGCAGAAGA
GGAATGTCATCAGGATCATCCCCCACCACAACTACAATGC
TGCCATCAACAAGTACAACCATGACATAGCCCTGCTGGAG
CTTGACAAGCCCCTGGTGCTCAACAGCTATGTGACCCCCA
TCTGTATAGCCAACAAGGAGTACACCAACATCTTCCTCAA
GTTTGGCTCAGGCTATGTGTCAGGCTGGGGCAGGGTGTTC
CACAGGGGCAGGTCAGCCTCTGTGCTGCAGTACCTGAGGG
TGCCCCTGGTTGACAGGGCCACCTGCCTGAGGAGCACCAA
GTTCACCATCTACAACAACATGTTCTGTGCTGGCTTCCAT
GAGGGGGGCAGGGACAGCTGCCAGGGTGACAGTGGGGGCC
CTCATGTGACAGAGGTTGAGGGCACCAGCTTCCTGACAGG
CATCATCAGCTGGGGGGAGGAGTGTGCCATGAAGGGCAAG
TATGGCATCTACACCAAGGTGAGCAGGTATGTCAACTGGA
TCAAGGAGAAGACCAAGCTGACATGA
FIX Optimized Sequence Gamma 3
(SEQ ID NO: 42)
ATGCAGAGAGTGAACATGATCATGGCTGAGTCACCTGGAC
TGATCACCATCTGCCTGCTGGGCTACCTGCTGTCTGCTGA
GTGCACAGTCTTCCTGGATCATGAGAATGCCAACAAGATC
CTGAACAGACCTAAGAGGTACAACTCTGGCAAGCTGGAGG
AGTTTGTGCAGGGCAACCTGGAGAGAGAGTGCATGGAGGA
GAAGTGCAGCTTTGAGGAGGCCAGAGAGGTGTTTGAGAAC
ACAGAGAGGACCACAGAGTTCTGGAAGCAGTATGTGGATG
GTGATCAGTGTGAGAGCAACCCCTGCCTGAATGGAGGCAG
CTGCAAGGATGATATCAACTCCTATGAGTGCTGGTGCCCC
TTTGGCTTTGAGGGCAAGAACTGTGAGCTGGATGCCACCT
GCAACATCAAGAATGGCAGATGTGAGCAGTTCTGCAAGAA
CAGTGCTGATAACAAGGTGGTGTGCAGCTGCACAGAGGGC
TACAGACTGGCTGAGAACCAGAAGAGCTGTGAGCCAGCTG
TGCCCTTCCCCTGTGGCAGAGTGTCAGTGTCACAGACCAG
CAAGCTGACCAGAGCTGAGACAGTCTTCCCTGATGTGGAC
TATGTGAACAGCACAGAGGCTGAGACCATCCTGGATAACA
TCACCCAGTCCACCCAGTCATTCAATGACTTCACCAGAGT
GGTGGGAGGAGAGGATGCCAAGCCTGGCCAGTTCCCCTGG
CAGGTGGTGCTGAATGGCAAGGTGGATGCATTCTGTGGAG
GCAGCATTGTGAATGAGAAGTGGATTGTGACAGCTGCCCA
CTGTGTGGAGACAGGTGTGAAGATCACAGTGGTGGCTGGA
GAGCACAACATTGAGGAGACAGAGCACACAGAGCAGAAGA
GGAATGTGATCAGGATCATCCCTCACCACAACTACAATGC
TGCCATCAACAAGTACAACCATGATATTGCACTGCTGGAG
CTGGATAAGCCACTGGTGCTGAACAGCTATGTGACACCTA
TCTGCATTGCCAACAAGGAGTACACCAACATCTTCCTGAA
GTTTGGCAGTGGCTATGTGTCAGGCTGGGGCAGAGTGTTC
CACAGAGGCAGATCAGCCTCTGTGCTGCAGTACCTGAGAG
TGCCACTGGTGGATAGAGCCACCTGCCTGAGGAGCACCAA
GTTCACCATCTACAACAACATGTTCTGTGCTGGCTTCCAT
GAGGGAGGCAGAGATAGCTGCCAGGGTGACAGTGGTGGCC
CTCATGTGACAGAGGTGGAGGGCACCTCCTTCCTGACTGG
CATCATCTCCTGGGGAGAGGAGTGTGCCATGAAGGGCAAG
TATGGCATCTACACCAAGGTGAGCAGATATGTGAACTGGA
TCAAGGAGAAGACCAAGCTGACATGA
FIX Optimized Sequence Gamma 4
(SEQ ID NO: 43)
ATGCAGAGAGTGAACATGATCATGGCTGAGAGCCCTGGCC
TGATCACCATCTGCCTGCTGGGCTACCTGCTGTCTGCTGA
GTGCACAGTGTTCCTGGATCATGAGAATGCCAACAAGATC
CTGAACAGACCCAAGAGATACAACTCTGGCAAGCTGGAGG
AGTTTGTGCAGGGCAACCTGGAGAGAGAGTGCATGGAGGA
GAAGTGCTCCTTTGAGGAGGCCAGAGAGGTGTTTGAGAAC
ACAGAGAGAACCACAGAGTTCTGGAAGCAGTATGTGGATG
GAGATCAGTGTGAGTCCAACCCTTGCCTGAATGGAGGCTC
CTGCAAGGATGATATCAACTCCTATGAGTGCTGGTGCCCT
TTTGGCTTTGAGGGCAAGAACTGTGAGCTGGATGCCACCT
GCAACATCAAGAATGGCAGATGTGAGCAGTTCTGCAAGAA
CTCTGCTGATAACAAGGTGGTGTGCTCCTGCACAGAGGGC
TACAGACTGGCTGAGAACCAGAAGTCCTGTGAGCCTGCTG
TGCCCTTCCCTTGTGGCAGAGTGTCTGTGTCCCAGACCTC
CAAGCTGACCAGAGCTGAGACAGTGTTCCCTGATGTGGAT
TATGTGAACTCCACAGAGGCTGAGACCATCCTGGATAACA
TCACCCAGTCCACCCAGTCCTTCAATGATTTCACCAGAGT
GGTGGGAGGAGAGGATGCCAAGCCTGGCCAGTTCCCTTGG
CAGGTGGTGCTGAATGGCAAGGTGGATGCCTTCTGTGGAG
GCTCCATTGTGAATGAGAAGTGGATTGTGACAGCTGCCCA
CTGTGTGGAGACAGGAGTGAAGATCACAGTGGTGGCTGGA
GAGCACAACATTGAGGAGACAGAGCACACAGAGCAGAAGA
GAAATGTGATCAGAATCATCCCTCACCACAACTACAATGC
TGCCATCAACAAGTACAACCATGATATTGCCCTGCTGGAG
CTGGATAAGCCTCTGGTGCTGAACTCCTATGTGACACCCA
TCTGCATTGCCAACAAGGAGTACACCAACATCTTCCTGAA
GTTTGGCTCTGGCTATGTGTCTGGCTGGGGCAGAGTGTTC
CACAGAGGCAGATCTGCCTCTGTGCTGCAGTACCTGAGAG
TGCCTCTGGTGGATAGAGCCACCTGCCTGAGATCCACCAA
GTTCACCATCTACAACAACATGTTCTGTGCTGGCTTCCAT
GAGGGAGGCAGAGATTCCTGCCAGGGAGATTCTGGAGGCC
CTCATGTGACAGAGGTGGAGGGCACCTCCTTCCTGACAGG
CATCATCTCCTGGGGAGAGGAGTGTGCCATGAAGGGCAAG
TATGGCATCTACACCAAGGTGTCCAGATATGTGAACTGGA
TCAAGGAGAAGACCAAGCTGACCTGA

FIX Optimized Sequences: (With Padua)

Liver Codon Optimized Sequences for fIX optimized sequence Gamma (with Padua), SEQ ID NO: 56 are SEQ ID NOS: 44-47.

Gamma-22 Padua
(SEQ ID NO: 44)
ATGCAGAGGGTCAACATGATCATGGCAGAGTCCCCTGGGC
TCATCACCATCTGCCTCCTGGGGTACCTGCTCAGTGCTGA
GTGCACAGTGTTCCTGGACCATGAGAATGCCAACAAGATC
CTGAACAGGCCCAAGAGGTACAACTCTGGGAAACTGGAGG
AGTTTGTCCAGGGGAACCTGGAGAGGGAGTGCATGGAGGA
GAAGTGCTCCTTTGAGGAGGCCAGGGAGGTCTTTGAGAAC
ACAGAGAGGACCACTGAGTTCTGGAAGCAGTATGTGGATG
GGGACCAGTGTGAGTCCAACCCCTGCCTCAATGGGGGCAG
CTGCAAGGATGACATCAACTCCTATGAGTGCTGGTGCCCC
TTTGGCTTTGAGGGCAAGAACTGTGAGCTGGATGCCACCT
GCAACATCAAGAATGGCAGATGTGAGCAGTTCTGCAAGAA
CTCTGCTGACAACAAGGTGGTGTGCAGCTGCACTGAGGGC
TACAGGCTGGCTGAGAACCAGAAGTCCTGTGAGCCAGCTG
TGCCCTTCCCCTGTGGCAGAGTCTCAGTGTCCCAGACCTC
CAAGCTGACCAGAGCTGAGACAGTGTTCCCAGATGTGGAC
TATGTGAACTCCACAGAGGCTGAGACCATCCTGGACAACA
TCACCCAGAGCACCCAGAGCTTCAATGACTTCACCAGGGT
GGTGGGAGGAGAAGATGCCAAGCCTGGGCAGTTCCCCTGG
CAGGTGGTGCTCAATGGCAAAGTGGATGCCTTCTGTGGAG
GCTCCATTGTCAATGAGAAGTGGATTGTCACAGCTGCCCA
CTGTGTGGAGACAGGGGTCAAGATCACAGTGGTGGCAGGG
GAGCACAACATTGAGGAGACAGAGCACACAGAGCAGAAGA
GGAATGTGATCAGGATCATCCCCCACCACAACTACAATGC
TGCCATCAACAAGTACAACCATGACATTGCCCTGCTGGAG
CTGGACAAGCCCCTGGTGCTGAACTCCTATGTGACCCCCA
TCTGCATTGCCAACAAGGAGTACACCAACATCTTCCTGAA
GTTTGGCTCTGGGTATGTCTCAGGCTGGGGCAGAGTGTTC
CACAGGGGCAGAAGTGCCTCAGTGCTGCAGTACCTCAGGG
TGCCCCTGGTGGACAGGGCCACTTGCCTCCTGAGCACCAA
GTTCACCATCTACAACAACATGTTCTGTGCAGGGTTCCAT
GAGGGGGGCAGGGACTCCTGCCAGGGGGACTCTGGGGGCC
CCCATGTCACAGAGGTGGAGGGCACCAGTTTCCTCACAGG
GATCATCTCCTGGGGTGAGGAGTGTGCCATGAAGGGCAAG
TATGGCATCTACACCAAGGTGTCCAGGTATGTGAACTGGA
TCAAGGAGAAGACCAAGCTGACCTGA
CAI: 0.8895259385925125
Energy: −553.8 kcal/mol
Original CAI: 0.88897, Energy: −557.5
Gamma 3 Padua
(SEQ ID NO: 45)
ATGCAGAGGGTCAACATGATCATGGCTGAGAGCCCTGGCC
TCATCACCATCTGCCTGCTGGGCTACCTGCTGTCAGCTGA
GTGCACAGTGTTCCTGGACCATGAGAATGCCAACAAGATC
CTCAACAGGCCCAAGAGGTACAACAGTGGCAAGCTGGAGG
AGTTTGTGCAGGGCAACCTGGAGAGGGAGTGCATGGAGGA
GAAGTGCAGCTTTGAGGAGGCCAGGGAGGTGTTTGAGAAC
ACAGAGAGGACAACAGAGTTCTGGAAGCAGTATGTTGATG
GTGACCAGTGTGAGAGCAACCCCTGCCTGAATGGGGGCAG
CTGCAAGGATGACATCAACAGCTATGAGTGCTGGTGCCCC
TTTGGCTTTGAGGGCAAGAACTGTGAGCTTGATGCCACCT
GCAACATCAAGAATGGCAGGTGTGAGCAGTTCTGCAAGAA
CAGTGCTGACAACAAGGTGGTGTGCAGCTGCACAGAGGGC
TACAGGCTGGCTGAGAACCAGAAGAGCTGTGAGCCTGCTG
TGCCCTTCCCCTGTGGCAGGGTGTCTGTGAGCCAGACAAG
CAAGCTGACCAGGGCTGAGACAGTGTTCCCTGATGTTGAC
TATGTCAACAGCACAGAGGCTGAGACCATCCTGGACAACA
TCACACAGTCAACACAGAGCTTCAATGACTTCACCAGGGT
GGTGGGGGGGGAGGATGCCAAGCCTGGCCAGTTCCCCTGG
CAGGTGGTGCTGAATGGCAAGGTTGATGCCTTCTGTGGGG
GCAGCATTGTCAATGAGAAGTGGATTGTGACAGCTGCCCA
CTGTGTTGAGACAGGGGTCAAGATCACAGTGGTGGCTGGT
GAGCACAACATTGAGGAGACAGAGCACACAGAGCAGAAGA
GGAATGTCATCAGGATCATCCCCCACCACAACTACAATGC
TGCCATCAACAAGTACAACCATGACATAGCCCTGCTGGAG
CTTGACAAGCCCCTGGTGCTCAACAGCTATGTGACCCCCA
TCTGTATAGCCAACAAGGAGTACACCAACATCTTCCTCAA
GTTTGGCTCAGGCTATGTGTCAGGCTGGGGCAGGGTGTTC
CACAGGGGCAGGTCAGCCTCTGTGCTGCAGTACCTGAGGG
TGCCCCTGGTTGACAGGGCCACCTGCCTGCTGAGCACCAA
GTTCACCATCTACAACAACATGTTCTGTGCTGGCTTCCAT
GAGGGGGGCAGGGACAGCTGCCAGGGTGACAGTGGGGGCC
CTCATGTGACAGAGGTTGAGGGCACCAGCTTCCTGACAGG
CATCATCAGCTGGGGGGAGGAGTGTGCCATGAAGGGCAAG
TATGGCATCTACACCAAGGTGAGCAGGTATGTCAACTGGA
TCAAGGAGAAGACCAAGCTGACATGA
CAI: 0.8842265792948748
Energy: −524.7 kcal/mol
Original CAI: 0.883674, energy: −524.1
Gamma 87 Padua
(SEQ ID NO: 46):
ATGCAGAGAGTGAACATGATCATGGCTGAGTCACCTGGAC
TGATCACCATCTGCCTGCTGGGCTACCTGCTGTCTGCTGA
GTGCACAGTCTTCCTGGATCATGAGAATGCCAACAAGATC
CTGAACAGACCTAAGAGGTACAACTCTGGCAAGCTGGAGG
AGTTTGTGCAGGGCAACCTGGAGAGAGAGTGCATGGAGGA
GAAGTGCAGCTTTGAGGAGGCCAGAGAGGTGTTTGAGAAC
ACAGAGAGGACCACAGAGTTCTGGAAGCAGTATGTGGATG
GTGATCAGTGTGAGAGCAACCCCTGCCTGAATGGAGGCAG
CTGCAAGGATGATATCAACTCCTATGAGTGCTGGTGCCCC
TTTGGCTTTGAGGGCAAGAACTGTGAGCTGGATGCCACCT
GCAACATCAAGAATGGCAGATGTGAGCAGTTCTGCAAGAA
CAGTGCTGATAACAAGGTGGTGTGCAGCTGCACAGAGGGC
TACAGACTGGCTGAGAACCAGAAGAGCTGTGAGCCAGCTG
TGCCCTTCCCCTGTGGCAGAGTGTCAGTGTCACAGACCAG
CAAGCTGACCAGAGCTGAGACAGTCTTCCCTGATGTGGAC
TATGTGAACAGCACAGAGGCTGAGACCATCCTGGATAACA
TCACCCAGTCCACCCAGTCATTCAATGACTTCACCAGAGT
GGTGGGAGGAGAGGATGCCAAGCCTGGCCAGTTCCCCTGG
CAGGTGGTGCTGAATGGCAAGGTGGATGCATTCTGTGGAG
GCAGCATTGTGAATGAGAAGTGGATTGTGACAGCTGCCCA
CTGTGTGGAGACAGGTGTGAAGATCACAGTGGTGGCTGGA
GAGCACAACATTGAGGAGACAGAGCACACAGAGCAGAAGA
GGAATGTGATCAGGATCATCCCTCACCACAACTACAATGC
TGCCATCAACAAGTACAACCATGATATTGCACTGCTGGAG
CTGGATAAGCCACTGGTGCTGAACAGCTATGTGACACCTA
TCTGCATTGCCAACAAGGAGTACACCAACATCTTCCTGAA
GTTTGGCAGTGGCTATGTGTCAGGCTGGGGCAGAGTGTTC
CACAGAGGCAGATCAGCCTCTGTGCTGCAGTACCTGAGAG
TGCCACTGGTGGATAGAGCCACCTGCCTGCTGAGCACCAA
GTTCACCATCTACAACAACATGTTCTGTGCTGGCTTCCAT
GAGGGAGGCAGAGATAGCTGCCAGGGTGACAGTGGTGGCC
CTCATGTGACAGAGGTGGAGGGCACCTCCTTCCTGACTGG
CATCATCTCCTGGGGAGAGGAGTGTGCCATGAAGGGCAAG
TATGGCATCTACACCAAGGTGAGCAGATATGTGAACTGGA
TCAAGGAGAAGACCAAGCTGACATGA
CAI: 0.9243302166940373
Energy: −512.2 kcal/mol
Original CAI: 0.923753, energy: −513.8
Gamma 2 Padua
(SEQ ID NO: 47)
ATGCAGAGAGTGAACATGATCATGGCTGAGAGCCCTGGCC
TGATCACCATCTGCCTGCTGGGCTACCTGCTGTCTGCTGA
GTGCACAGTGTTCCTGGATCATGAGAATGCCAACAAGATC
CTGAACAGACCCAAGAGATACAACTCTGGCAAGCTGGAGG
AGTTTGTGCAGGGCAACCTGGAGAGAGAGTGCATGGAGGA
GAAGTGCTCCTTTGAGGAGGCCAGAGAGGTGTTTGAGAAC
ACAGAGAGAACCACAGAGTTCTGGAAGCAGTATGTGGATG
GAGATCAGTGTGAGTCCAACCCTTGCCTGAATGGAGGCTC
CTGCAAGGATGATATCAACTCCTATGAGTGCTGGTGCCCT
TTTGGCTTTGAGGGCAAGAACTGTGAGCTGGATGCCACCT
GCAACATCAAGAATGGCAGATGTGAGCAGTTCTGCAAGAA
CTCTGCTGATAACAAGGTGGTGTGCTCCTGCACAGAGGGC
TACAGACTGGCTGAGAACCAGAAGTCCTGTGAGCCTGCTG
TGCCCTTCCCTTGTGGCAGAGTGTCTGTGTCCCAGACCTC
CAAGCTGACCAGAGCTGAGACAGTGTTCCCTGATGTGGAT
TATGTGAACTCCACAGAGGCTGAGACCATCCTGGATAACA
TCACCCAGTCCACCCAGTCCTTCAATGATTTCACCAGAGT
GGTGGGAGGAGAGGATGCCAAGCCTGGCCAGTTCCCTTGG
CAGGTGGTGCTGAATGGCAAGGTGGATGCCTTCTGTGGAG
GCTCCATTGTGAATGAGAAGTGGATTGTGACAGCTGCCCA
CTGTGTGGAGACAGGAGTGAAGATCACAGTGGTGGCTGGA
GAGCACAACATTGAGGAGACAGAGCACACAGAGCAGAAGA
GAAATGTGATCAGAATCATCCCTCACCACAACTACAATGC
TGCCATCAACAAGTACAACCATGATATTGCCCTGCTGGAG
CTGGATAAGCCTCTGGTGCTGAACTCCTATGTGACACCCA
TCTGCATTGCCAACAAGGAGTACACCAACATCTTCCTGAA
GTTTGGCTCTGGCTATGTGTCTGGCTGGGGCAGAGTGTTC
CACAGAGGCAGATCTGCCTCTGTGCTGCAGTACCTGAGAG
TGCCTCTGGTGGATAGAGCCACCTGCCTGCTGTCCACCAA
GTTCACCATCTACAACAACATGTTCTGTGCTGGCTTCCAT
GAGGGAGGCAGAGATTCCTGCCAGGGAGATTCTGGAGGCC
CTCATGTGACAGAGGTGGAGGGCACCTCCTTCCTGACAGG
CATCATCTCCTGGGGAGAGGAGTGTGCCATGAAGGGCAAG
TATGGCATCTACACCAAGGTGTCCAGATATGTGAACTGGA
TCAAGGAGAAGACCAAGCTGACCTGA
CAI: 0.9439100225127741
Energy: −498.7 kcal/mol
Original CAI: 0.94391, energy: −495

Liver Codon Optimized Sequences for fIX optimized sequence Beta (with Padua), SEQ ID NO: 57 are SEQ ID NOS: 48-51.

Beta 82 Padua
(SEQ ID NO: 48)
ATGCAGAGGGTGAACATGATCATGGCTGAGAGCCCTGGGC
TGATCACCATCTGCCTGCTGGGGTACCTGCTGAGTGCTGA
GTGCACTGTGTTCCTGGACCATGAGAATGCCAACAAGATC
CTCAACAGGCCCAAGAGGTACAACAGTGGCAAGCTGGAGG
AGTTTGTGCAGGGCAACCTGGAGAGGGAGTGCATGGAGGA
GAAGTGCAGCTTTGAGGAGGCCAGGGAGGTGTTTGAGAAC
ACAGAGAGGACCACAGAGTTCTGGAAGCAGTATGTGGATG
GGGACCAGTGTGAGTCCAACCCCTGCCTGAATGGGGGCAG
CTGCAAGGATGACATCAACAGCTATGAGTGCTGGTGCCCC
TTTGGCTTTGAGGGCAAGAACTGTGAGCTGGATGCCACCT
GCAACATCAAGAATGGCAGGTGTGAGCAGTTCTGCAAGAA
CAGTGCTGACAACAAGGTGGTGTGCAGCTGCACTGAGGGC
TACAGGCTGGCTGAGAACCAGAAGTCCTGTGAGCCTGCAG
TGCCCTTCCCCTGTGGCAGGGTGTCTGTGTCCCAGACCAG
CAAGCTCACCAGGGCTGAGACTGTGTTCCCTGATGTGGAC
TATGTCAACAGCACAGAGGCTGAGACCATCCTGGACAACA
TCACCCAGAGCACCCAGAGCTTCAATGACTTCACCAGGGT
GGTGGGGGGGGAGGATGCCAAGCCAGGGCAGTTCCCCTGG
CAGGTGGTGCTGAATGGCAAGGTGGATGCCTTCTGTGGGG
GCAGCATTGTCAATGAGAAGTGGATTGTCACAGCAGCCCA
CTGTGTGGAGACTGGGGTGAAGATCACAGTGGTGGCTGGG
GAGCACAACATTGAGGAGACAGAGCACACAGAGCAGAAGA
GGAATGTCATCAGGATCATCCCCCACCACAACTACAATGC
TGCCATCAACAAGTACAACCATGACATTGCCCTGCTGGAG
CTGGATGAGCCCCTGGTGCTGAACAGCTATGTCACCCCCA
TCTGCATTGCCAACAAGGAGTACACCAACATCTTCCTGAA
GTTTGGCAGTGGCTATGTCAGTGGCTGGGGCAGGGTGTTC
CACAGGGGCAGGTCTGCCAGTGTGCTGCAGTACCTGAGGG
TGCCCCTGGTGGACAGGGCCACCTGCCTGCTGTCCACCAA
GTTCACCATCTACAACAACATGTTCTGTGCTGGCTTCCAT
GAGGGGGGCAGGGACAGCTGCCAGGGGGACTCTGGGGGCC
CCCATGTCACTGAGGTGGAGGGCACCAGCTTCCTGACAGG
CATCATCAGCTGGGGGGAGGAGTGTGCCATGAAGGGCAAG
TATGGCATCTACACCAAGGTCAGCAGGTATGTGAACTGGA
TCAAGGAGAAGACCAAGCTCACCTGA
CAI: 0.899728869695304
Energy: −546.3 kcal/mol
Original CAI: 0.899167, energy: −542.6
Beta 81 Padua
(SEQ ID NO: 49)
ATGCAGAGGGTGAACATGATCATGGCTGAGTCCCCTGGCC
TGATCACCATCTGCCTGCTGGGCTACCTGCTGTCTGCTGA
GTGCACAGTGTTCCTGGACCATGAGAATGCCAACAAGATC
CTGAACAGGCCCAAGAGGTACAACTCTGGCAAGCTGGAGG
AGTTTGTGCAGGGCAACCTGGAGAGGGAGTGCATGGAGGA
GAAGTGCTCCTTTGAGGAGGCCAGGGAGGTGTTTGAGAAC
ACAGAGAGGACCACAGAGTTCTGGAAGCAGTATGTGGATG
GAGACCAGTGTGAGTCCAACCCTTGCCTGAATGGAGGCTC
CTGCAAGGATGACATCAACTCCTATGAGTGCTGGTGCCCC
TTTGGCTTTGAGGGCAAGAACTGTGAGCTGGATGCCACCT
GCAACATCAAGAATGGCAGGTGTGAGCAGTTCTGCAAGAA
CTCTGCTGACAACAAGGTGGTGTGCTCCTGCACAGAGGGC
TACAGGCTGGCTGAGAACCAGAAGTCCTGTGAGCCAGCTG
TGCCCTTCCCTTGTGGCAGGGTGTCTGTGTCCCAGACCTC
CAAGCTGACCAGGGCTGAGACAGTGTTCCCTGATGTGGAC
TATGTGAACTCCACAGAGGCTGAGACCATCCTGGACAACA
TCACCCAGTCCACCCAGTCCTTCAATGACTTCACCAGGGT
GGTGGGAGGTGAGGATGCCAAGCCTGGCCAGTTCCCTTGG
CAGGTGGTGCTGAATGGCAAGGTGGATGCCTTCTGTGGAG
GCTCCATTGTGAATGAGAAGTGGATTGTGACAGCTGCCCA
CTGTGTGGAGACAGGAGTGAAGATCACAGTGGTGGCTGGT
GAGCACAACATTGAGGAGACAGAGCACACAGAGCAGAAGA
GGAATGTGATCAGGATCATCCCCCACCACAACTACAATGC
TGCCATCAACAAGTACAACCATGACATTGCCCTGCTGGAG
CTGGATGAGCCCCTGGTGCTGAACTCCTATGTGACCCCCA
TCTGCATTGCCAACAAGGAGTACACCAACATCTTCCTGAA
GTTTGGCTCTGGCTATGTGTCAGGATGGGGCAGGGTGTTC
CACAGGGGCAGGTCTGCCTCTGTGCTGCAGTACCTGAGGG
TGCCCCTGGTGGACAGGGCCACCTGCCTGCTGTCCACCAA
GTTCACCATCTACAACAACATGTTCTGTGCTGGCTTCCAT
GAGGGAGGCAGGGACTCCTGCCAGGGTGACTCAGGAGGCC
CCCATGTGACAGAGGTGGAGGGCACCTCCTTCCTGACAGG
CATCATCAGCTGGGGAGAGGAGTGTGCCATGAAGGGCAAG
TATGGCATCTACACCAAGGTGTCCAGGTATGTGAACTGGA
TCAAGGAGAAGACCAAGCTGACCTGA
CAI: 0.9297160280666142
Energy: −528.7 kcal/mol
Original CAI: 0.929135, energy: −526.4
Beta 23 Padua
(SEQ ID NO: 50)
ATGCAGAGAGTCAACATGATCATGGCAGAGTCACCTGGAC
TGATCACCATCTGCCTGCTGGGCTACCTGCTCAGTGCAGA
GTGCACAGTCTTCCTGGACCATGAGAATGCCAACAAGATC
CTGAACAGACCCAAGAGGTACAACTCTGGCAAGCTGGAGG
AGTTTGTGCAGGGCAACCTGGAGAGAGAGTGCATGGAGGA
GAAGTGCTCCTTTGAGGAGGCCAGAGAGGTCTTTGAGAAC
ACTGAGAGGACCACTGAGTTCTGGAAGCAGTATGTGGATG
GTGATCAGTGTGAGTCCAACCCATGCCTGAATGGAGGCTC
CTGCAAGGATGACATCAACTCCTATGAGTGCTGGTGCCCA
TTTGGCTTTGAGGGCAAGAACTGTGAGCTGGATGCAACAT
GCAACATCAAGAATGGCAGATGTGAGCAGTTCTGCAAGAA
CAGTGCAGACAACAAGGTGGTGTGCTCCTGCACAGAGGGC
TACAGACTGGCTGAGAACCAGAAGTCATGTGAGCCTGCTG
TGCCCTTCCCCTGTGGCAGAGTGAGTGTGTCCCAGACCAG
CAAGCTGACCAGAGCTGAGACAGTCTTCCCTGATGTGGAC
TATGTGAACTCCACAGAGGCAGAGACCATCCTGGACAACA
TCACACAGTCCACACAGTCCTTCAATGACTTCACCAGAGT
GGTTGGTGGTGAGGATGCCAAGCCTGGACAGTTCCCCTGG
CAGGTGGTGCTCAATGGCAAGGTGGATGCCTTCTGTGGAG
GCTCCATTGTCAATGAGAAGTGGATTGTCACAGCTGCCCA
CTGTGTGGAGACTGGTGTGAAGATCACTGTGGTGGCTGGT
GAGCACAACATTGAGGAGACAGAGCACACTGAGCAGAAGA
GGAATGTCATCAGGATCATCCCACACCACAACTACAATGC
AGCCATCAACAAGTACAACCATGACATTGCCCTGCTGGAG
CTGGATGAGCCCCTGGTGCTGAACAGCTATGTGACCCCCA
TCTGCATAGCCAACAAGGAGTACACCAACATCTTCCTGAA
GTTTGGCTCTGGCTATGTGTCTGGCTGGGGCAGAGTCTTC
CACAGAGGCAGATCTGCCTCTGTGCTGCAGTACCTGAGAG
TGCCTCTGGTGGACAGAGCAACCTGCCTGCTGAGCACCAA
GTTCACCATCTACAACAACATGTTCTGTGCAGGCTTCCAT
GAGGGAGGCAGAGACTCCTGCCAGGGAGACTCTGGTGGGC
CACATGTGACAGAGGTGGAGGGCACATCCTTCCTGACAGG
CATCATCTCCTGGGGAGAGGAGTGTGCCATGAAGGGCAAG
TATGGCATCTACACCAAGGTGTCCAGGTATGTCAACTGGA
TCAAGGAGAAGACCAAGCTGACCTGA
CAI: 0.9044489593184231
Energy: −518.4 kcal/mol
Original CAI: 0.904449, energy: −517.1
Beta 39 Padua
(SEQ ID NO: 51)
ATGCAGAGAGTGAACATGATCATGGCTGAGTCCCCTGGCC
TGATCACCATCTGCCTGCTGGGCTACCTGCTGTCTGCTGA
GTGCACAGTGTTCCTGGATCATGAGAATGCCAACAAGATC
CTGAACAGACCCAAGAGATACAACTCTGGCAAGCTGGAGG
AGTTTGTGCAGGGCAACCTGGAGAGAGAGTGCATGGAGGA
GAAGTGCTCCTTTGAGGAGGCCAGAGAGGTGTTTGAGAAC
ACAGAGAGAACCACAGAGTTCTGGAAGCAGTATGTGGATG
GAGATCAGTGTGAGTCCAACCCTTGCCTGAATGGAGGCTC
CTGCAAGGATGATATCAACTCCTATGAGTGCTGGTGCCCA
TTTGGCTTTGAGGGCAAGAATTGTGAGCTGGATGCCACCT
GCAACATCAAGAATGGCAGATGTGAGCAGTTCTGCAAGAA
CTCTGCTGATAACAAGGTGGTGTGCTCCTGCACAGAGGGC
TACAGACTGGCTGAGAACCAGAAGTCCTGTGAGCCTGCTG
TGCCTTTCCCTTGTGGCAGAGTGTCTGTGTCCCAGACCTC
CAAGCTGACCAGAGCTGAGACAGTGTTCCCTGATGTGGAT
TATGTGAACTCCACAGAGGCTGAGACCATCCTGGATAACA
TCACCCAGTCCACCCAGTCCTTCAATGATTTCACCAGAGT
GGTGGGAGGAGAGGATGCCAAGCCTGGCCAGTTCCCCTGG
CAGGTGGTGCTGAATGGCAAGGTGGATGCCTTCTGTGGAG
GCTCCATTGTGAATGAGAAGTGGATTGTGACAGCTGCCCA
CTGTGTGGAGACAGGAGTGAAGATCACAGTGGTGGCTGGA
GAGCACAACATTGAGGAGACAGAGCACACAGAGCAGAAGA
GAAATGTGATCAGAATCATCCCTCACCACAACTACAATGC
TGCCATCAACAAGTACAACCATGATATTGCCCTGCTGGAG
CTGGATGAGCCCCTGGTGCTGAACTCCTATGTGACCCCTA
TCTGCATTGCCAACAAGGAGTACACCAACATCTTCCTGAA
GTTTGGCTCTGGCTATGTGTCTGGCTGGGGCAGAGTGTTC
CACAGAGGCAGATCTGCCTCTGTGCTGCAGTACCTGAGAG
TGCCTCTGGTGGATAGAGCCACCTGCCTGCTGTCCACCAA
GTTCACCATCTACAACAACATGTTCTGTGCTGGCTTCCAT
GAGGGAGGCAGAGATTCCTGCCAGGGAGATTCTGGAGGCC
CTCATGTGACAGAGGTGGAGGGCACCTCCTTCCTGACAGG
CATCATCTCCTGGGGAGAGGAGTGTGCCATGAAGGGCAAG
TATGGCATCTACACCAAGGTGTCCAGATATGTGAACTGGA
TCAAGGAGAAGACCAAGCTGACCTGA
CAI: 0.944126528656901
Energy: −493.1 kcal/mol
Original CAI: 0.944127, energy: −489.4

### Example 5

### Treatment of human hemophilia B using AAV-based gene therapy

This example describes an exemplary method for the clinical use of AAV vectors encoding fIX for the treatment of hemophilia B.

A patient diagnosed with hemophilia B is selected for treatment. The patient is administered a therapeutically effective amount of a recombinant AAV encoding the An96 fIX Padua variant (e.g., SEQ ID NO: 17) under control of a HCB promoter. The recombinant AAV can be administered intravenously. An appropriate therapeutic dose can be selected by a medical practitioner. In some cases, the therapeutically effective dose is in the range of 1×10¹¹ to 1×10¹⁴ viral particles (vp)/kg, such as about 1×10¹² vp/kg. In most instances, the patient is administered a single dose. The health of the subject can be monitored over time to determine the effectiveness of the treatment.

### Example 6

### Treatment of human hemophilia B using AAV-based gene therapy

This example describes an exemplary method for the clinical use of AAV vectors encoding fIX for the treatment of hemophilia B.

Huh-7 liver cells were transiently transfected with equal amounts of AAV2 expression plasmids containing fIX constructs, 24 hours post-transfection, medium was exchanged to AIM-V serum reduced medium, and fIX expression measured by one-stage APTT-dependent coagulation assay from conditioned medium 24 hours after AIM-V medium exchange. FIX activity (units/24 hr/10⁶ cells) was compared to human fIX and An96

Additionally, in vivo studies were performed in hemophilia B mouse model using AAV2/8 vectors comparing AAV2-fIX-V86A-PRO2 (Group A) and AAV2-fIX-V86A-PRO2B (Group B). Data supports stable plasma fIX expression at therapeutics levels. See FIG. 8.

It will be apparent that the precise details of the methods or compositions described may be varied or modified without departing from the spirit of the described embodiments. We claim all such modifications and variations that fall within the scope and spirit of the claims below.

We claim:

1. A modified FIX polypeptide comprising an amino acid replacement corresponding to V132A-V86A in an unmodified FIX polypeptide, wherein:
corresponding amino acid residues are identified by alignment of the unmodified FIX polypeptide with the polypeptide of SEQ ID NO:1 or SEQ ID NO: 15, or a sequence of amino acid residues having at least 95% sequence identity to the FIX polypeptide sequence set forth in any of SEQ ID NO: 1, SEQ ID NO: 15;
the modified FIX polypeptide, when an active form, exhibits one or both of increased catalytic activity and increased procoagulant activity compared with the unmodified FIX polypeptide.

2. The modified FIX polypeptide of claim 1, further comprising at least one amino acid replacements selected from among N313S-N267S; E323K-E277K; V326T-V280T; D338N-D292N; K339R-K293R; H361N-H315N; K362R-K316R; L367S-L321S; L366S-L320S;
V368I-V322I-V367I-V321I-F399Y-F353Y; and R404K-R358K.

3. The modified FIX polypeptide of claim 2, further comprising at least one amino acid replacement selected from among E323K-E277K; V326T-V280T; D338N-D292N; K339R-K293R;
K362R-K316R; and L367S-L321S.

4. The modified FIX polypeptide of claim 1, further comprising at least one amino acid replacements selected from among D232N-D186N; V243L-V197; V248I-V242I-V257I-V211I; I262V-I216V; V269I-V223I-T271P-T225P; E286K-E240K; H289P- H243P;
I299V-I253V; A308T-A262T.

5. The modified FIX polypeptide of claim 3, further comprising at least one amino acid replacements selected from among D338N-D292N; K362R-K316R; and L367S-L321S.

6. The modified FIX polypeptide of claim 3, further comprising at least one amino acid replacements selected from among R384E-R338E and R384L-R338L.

7. The modified FIX polypeptide of claim 1, wherein the unmodified FIX polypeptide consists of a sequence of amino acids set forth in any of SEQ ID NOS: 52 through 57.

8. The modified FIX polypeptide of claim 1 that comprises one or more modifications selected from a chemical modification or a post-translational modification, wherein the modified FIX polypeptide is glycosylated, carboxylated, hydroxylated, sulfated, phosphorylated, albuminated, or conjugated to a polyethylene glycol (PEG) moiety.

9. A pharmaceutical composition, comprising the modified FIX polypeptide of claim 1, in a pharmaceutically acceptable vehicle.

10. The pharmaceutical composition of claim 9 that is formulated for local, systemic, or topical administration.

11. The pharmaceutical composition of claim 9 that is formulated for oral, nasal, pulmonary, buccal, transdermal, subcutaneous, intraduodenal, enteral, parenteral, intravenous, or intramuscular administration.

12. The pharmaceutical composition of claim 9 that is formulated for controlled-release.

13. The pharmaceutical composition of claim 9 that is formulated for single-dosage administration.

14. The modified FIX polypeptide of claim 1 that is glycosylated.

15. The modified FIX polypeptide of claim 1 that is hyperglycosylated.

16. The modified FIX polypeptide of claim 1 that is a mature polypeptide.

17. The modified FIX polypeptide of claim 1 that is activated.

18. The modified FIX polypeptide of claim 1 that is a zymogen.

19. A method, comprising treating a subject by administering the pharmaceutical composition of claim 1, wherein the subject has a disease or condition that is treated by administration of FIX or a procoagulant.

20. The method of claim 19, wherein the disease or condition to be treated is selected from among blood coagulation disorders, hematologic disorders, hemorrhagic disorders, hemophilias, and bleeding disorders.


SEQUENCE LISTING (57 sequences)

SEQ ID NO: 1 - protein, 461 aa, Homo sapiens
  SITE (1)..(461): Human fIX
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFNKGRSASVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 2 - protein, 462 aa, Artificial Sequence
  Synthetic An96 fIX
MQCLNMIMAESPGLITICLLGYLLSAECTVFLDHENATKILNRPKRYNSGKLEEFVRGNLERECIEEKCSFEEAREVFENTEKTTEFWKQYVDGDQCESNPCLNGGSCKDDINSYECWCRFGFEGKNCELDATCSIKNGRCKQFCKKSADNKVVCSCTEGYRLAEDQKSCEPAVPFPCGRVSVSHTSKKLTRAETIFSNMDYENSTEAETILDNVTQSTQSFNDFTRVVGGENAKPGQFPWQVLLNGKIDAFCGGSIINEKWVVTAAHCIEPGVKITVVAGEHNIEKTEPTEQKRNVIRVIPHHNYNATINKYSHDIALLELDKPLTLNSYVTPICIANREYTNIFLKFGSGYVSGWGRVFNRGRSASILQYLRVPLVDRATCLRSTKFTIYNNMFCAGYHEGGKDSCQGDSGGPHVTEVEGTSFLTGIISWGEECAMKGKYGIYTKVSRYVNWIKEKTKLT

SEQ ID NO: 3 - DNA, 1386 bp, Artificial Sequence
  Synthetic hfIX-96sp3pro - hfIX with An96 Signal Peptide, Propetide, and Protease Domains
atgcagtgcctgaacatgatcatggccgagtcccccggcctgatcaccatctgcctgctggggtacctgctgagcgccgagtgcaccgtgttcctggaccacgagaacgccaccaagatcctgaacaggcccaagagatacaattctggaaagctggaggaatttgtgcagggcaacctggagagggaatgcatggaggaaaagtgtagctttgaggaagctagggaggtgtttgaaaacacagagaggaccacagaattctggaagcagtatgtggatggagatcagtgtgagtccaacccctgtctgaatggagggtcttgcaaagatgatatcaactcctatgagtgctggtgtccttttggatttgaaggcaaaaattgtgagctggatgtgacctgtaacatcaagaatggcaggtgtgagcagttctgtaaaaactctgctgataataaggtggtctgcagctgtacagaaggctataggctggctgagaaccagaagagctgtgaaccagctgtgcccttcccttgtgggagggtgtctgtcagccagacctctaagctgaccagagctgagactgtgttcccagatgtggattatgtcaactccacagaggctgaaaccatcctggacaacatcacccagtctacccagtccttcaatgactttaccagagtggtgggaggagagaacgctaagccaggacagttcccatggcaggtgctgctgaacgggaagatcgacgccttctgcggcgggtccatcatcaacgagaagtgggtggtgaccgctgctcactgcatcgagccaggagtgaagatcaccgtggtggctggggagcacaacatcgagaagaccgagcccaccgagcagaagcgcaacgtgatccgcgtgatcccccaccacaactacaacgccaccatcaacaagtactcccacgacatcgccctgctggagctggacaagcccctgaccctgaacagctacgtgacccccatctgcatcgccaacagggagtacaccaacatcttcctgaagttcggatccggatacgtgagcggatggggacgcgtgttcaaccgcggccggtccgccagcatcctgcagtacctgagagtgccactggtggacagagctacctgcctgcggtccaccaagttcaccatctacaacaacatgttctgcgctggataccacgagggagggaaggactcctgccagggggacagcggaggaccacacgtgaccgaggtggagggcacctccttcctgaccggcatcatcagctggggggaggagtgcgccatgaagggcaagtacgggatctacaccaaggtgagcagatacgtgaactggatcaaggagaagaccaagctgacctga

SEQ ID NO: 4 - DNA, 1389 bp, Artificial Sequence
  Synthetic An96-hfIXpro - An96 with hfIX Protease Domain
atgcagtgcctgaacatgatcatggccgagtcccccggcctgatcaccatctgcctgctggggtacctgctgagcgccgagtgcaccgtgttcctggaccacgagaacgccaccaagatcctgaacaggcccaagagatacaactccggcaagctggaggagttcgtgagggggaacctggagagagagtgcatcgaggagaagtgcagcttcgaggaggccagggaggtgttcgagaacaccgagaagaccaccgagttctggaagcagtacgtggacggcgaccagtgcgagtccaacccctgcctgaacggcgggtcctgcaaggacgacatcaacagctacgagtgctggtgcaggttcggcttcgaggggaagaactgcgagctggacgccacctgcagcatcaagaacggcagatgcaagcagttctgcaagaagtccgccgacaacaaggtggtgtgcagctgcaccgagggatacagactggctgaggaccagaagtcctgcgagccagctgtgccattcccatgcgggagggtgtccgtgagccacaccagcaagaagctgaccagagccgaaaccatcttctccaacatggactacgagaacagcaccgaggccgaaaccatcctggacaacgtgacccagtccacccagagcttcaacgacttcacccgggtggtgggaggagaggatgccaaaccaggccagttcccctggcaggtggtcctgaatgggaaggtggatgctttttgtgggggatccattgtgaatgagaaatggattgtcacagctgctcactgtgtggagacaggggtcaagatcactgtggtggctggagagcacaacattgaggaaacagaacatactgagcagaagaggaatgtgatcagaatcatccctcaccataactacaatgctgctatcaacaaatataatcatgacattgccctgctggaactggatgagcctctggtgctgaacagctatgtcaccccaatctgcattgctgacaaagagtataccaatatcttcctgaagtttggatctggatatgtgtctggatggggaagggtcttccacaagggcaggtctgccctggtgctgcagtatctgagggtgcctctggtggacagagctacctgcctgaggtctaccaagttcaccatctacaacaatatgttctgtgctggatttcatgagggaggcagggactcctgtcagggggattctggaggcccacatgtgacagaggtggaaggcaccagcttcctgactggcatcatctcttggggggaggaatgtgctatgaaggggaaatatggaatctacaccaaagtgagcaggtatgtgaactggatcaaagagaagaccaaactgacctga

SEQ ID NO: 5 - DNA, 1386 bp, Artificial Sequence
  Synthetic hfIX-96appro - hfIX with An96 Activation Peptide and Protease Domains
atgcagagggtcaatatgatcatggctgaatctcctgggctgatcaccatttgcctgctgggatacctgctgtctgctgagtgtacagtgttcctggaccatgagaatgccaataagatcctgaacaggcccaaaagatacaattctggaaagctggaggaatttgtgcagggcaacctggagagggaatgcatggaggaaaagtgtagctttgaggaagctagggaggtgtttgaaaacacagagaggaccacagaattctggaagcagtatgtggatggagatcagtgtgagtccaacccctgtctgaatggagggtcttgcaaagatgatatcaactcctatgagtgctggtgtccttttggatttgaaggcaaaaattgtgagctggatgtgacctgtaacatcaagaatggcaggtgtgagcagttctgtaaaaactctgctgataataaggtggtctgcagctgtacagaaggctataggctggctgagaaccagaagagctgtgaaccagctgtgcccttcccttgtgggagggtgtctgtcagccagacctctaagctgaccagagccgaaaccatcttctccaacatggactacgagaacagcaccgaggccgaaaccatcctggacaacgtgacccagtccacccagagcttcaacgacttcacccgggtggtgggaggagagaacgctaagccaggacagttcccatggcaggtgctgctgaacgggaagatcgacgccttctgcggcgggtccatcatcaacgagaagtgggtggtgaccgctgctcactgcatcgagccaggagtgaagatcaccgtggtggctggggagcacaacatcgagaagaccgagcccaccgagcagaagcgcaacgtgatccgcgtgatcccccaccacaactacaacgccaccatcaacaagtactcccacgacatcgccctgctggagctggacaagcccctgaccctgaacagctacgtgacccccatctgcatcgccaacagggagtacaccaacatcttcctgaagttcggatccggatacgtgagcggatggggacgcgtgttcaaccgcggccggtccgccagcatcctgcagtacctgagagtgccactggtggacagagctacctgcctgcggtccaccaagttcaccatctacaacaacatgttctgcgctggataccacgagggagggaaggactcctgccagggggacagcggaggaccacacgtgaccgaggtggagggcacctccttcctgaccggcatcatcagctggggggaggagtgcgccatgaagggcaagtacgggatctacaccaaggtgagcagatacgtgaactggatcaaggagaagaccaagctgacctga

SEQ ID NO: 6 - DNA, 1386 bp, Artificial Sequence
  Synthetic hfIX-96e2pro - hfIX with An96 EGF2 and Protease Domains
atgcagagggtcaatatgatcatggctgaatctcctgggctgatcaccatttgcctgctgggatacctgctgtctgctgagtgtacagtgttcctggaccatgagaatgccaataagatcctgaacaggcccaaaagatacaattctggaaagctggaggaatttgtgcagggcaacctggagagggaatgcatggaggaaaagtgtagctttgaggaagctagggaggtgtttgaaaacacagagaggaccacagaattctggaagcagtatgtggatggagatcagtgtgagtccaacccctgtctgaatggagggtcttgcaaagatgatatcaactcctatgagtgctggtgtccttttggatttgaaggcaaaaattgtgagctggacgccacctgcagcatcaagaacggcagatgcaagcagttctgcaagaagtccgccgacaacaaggtggtgtgcagctgcaccgagggatacagactggctgaggaccagaagtcctgcgagccagctgtgcccttcccttgtgggagggtgtctgtcagccagacctctaagctgaccagagctgagactgtgttcccagatgtggattatgtcaactccacagaggctgaaaccatcctggacaacatcacccagtctacccagtccttcaatgactttaccagagtggtgggaggagagaacgctaagccaggacagttcccatggcaggtgctgctgaacgggaagatcgacgccttctgcggcgggtccatcatcaacgagaagtgggtggtgaccgctgctcactgcatcgagccaggagtgaagatcaccgtggtggctggggagcacaacatcgagaagaccgagcccaccgagcagaagcgcaacgtgatccgcgtgatcccccaccacaactacaacgccaccatcaacaagtactcccacgacatcgccctgctggagctggacaagcccctgaccctgaacagctacgtgacccccatctgcatcgccaacagggagtacaccaacatcttcctgaagttcggatccggatacgtgagcggatggggacgcgtgttcaaccgcggccggtccgccagcatcctgcagtacctgagagtgccactggtggacagagctacctgcctgcggtccaccaagttcaccatctacaacaacatgttctgcgctggataccacgagggagggaaggactcctgccagggggacagcggaggaccacacgtgaccgaggtggagggcacctccttcctgaccggcatcatcagctggggggaggagtgcgccatgaagggcaagtacgggatctacaccaaggtgagcagatacgtgaactggatcaaggagaagaccaagctgacctga

SEQ ID NO: 7 - DNA, 1386 bp, Artificial Sequence
  Synthetic hfIX-96e2V86A - hfIX containing EGF2 V86A Amino Acid Substitution from An96
atgcagagggtcaatatgatcatggctgaatctcctgggctgatcaccatttgcctgctgggatacctgctgtctgctgagtgtacagtgttcctggaccatgagaatgccaataagatcctgaacaggcccaaaagatacaattctggaaagctggaggaatttgtgcagggcaacctggagagggaatgcatggaggaaaagtgtagctttgaggaagctagggaggtgtttgaaaacacagagaggaccacagaattctggaagcagtatgtggatggagatcagtgtgagtccaacccctgtctgaatggagggtcttgcaaagatgatatcaactcctatgagtgctggtgtccttttggatttgaaggcaaaaattgtgagctggatgccacctgtaacatcaagaatggcaggtgtgagcagttctgtaaaaactctgctgataataaggtggtctgcagctgtacagaaggctataggctggctgagaaccagaagagctgtgaaccagctgtgcccttcccttgtgggagggtgtctgtcagccagacctctaagctgaccagagctgagactgtgttcccagatgtggattatgtcaactccacagaggctgaaaccatcctggacaacatcacccagtctacccagtccttcaatgactttaccagagtggtgggaggagaggatgccaaaccaggccagttcccctggcaggtggtcctgaatgggaaggtggatgctttttgtgggggatccattgtgaatgagaaatggattgtcacagctgctcactgtgtggagacaggggtcaagatcactgtggtggctggagagcacaacattgaggaaacagaacatactgagcagaagaggaatgtgatcagaatcatccctcaccataactacaatgctgctatcaacaaatataatcatgacattgccctgctggaactggatgagcctctggtgctgaacagctatgtcaccccaatctgcattgctgacaaagagtataccaatatcttcctgaagtttggatctggatatgtgtctggatggggaagggtcttccacaagggcaggtctgccctggtgctgcagtatctgagggtgcctctggtggacagagctacctgcctgaggtctaccaagttcaccatctacaacaatatgttctgtgctggatttcatgagggaggcagggactcctgtcagggggattctggaggcccacatgtgacagaggtggaaggcaccagcttcctgactggcatcatctcttggggggaggaatgtgctatgaaggggaaatatggaatctacaccaaagtgagcaggtatgtgaactggatcaaagagaagaccaaactgacctga

SEQ ID NO: 8 - DNA, 1386 bp, Artificial Sequence
  Synthetic hfIX-96e2V86Apro2 - hfIX containing EGF2 V86A Amino Acid Substitution and Pro2 from An96
atgcagagggtcaatatgatcatggctgaatctcctgggctgatcaccatttgcctgctgggatacctgctgtctgctgagtgtacagtgttcctggaccatgagaatgccaataagatcctgaacaggcccaaaagatacaattctggaaagctggaggaatttgtgcagggcaacctggagagggaatgcatggaggaaaagtgtagctttgaggaagctagggaggtgtttgaaaacacagagaggaccacagaattctggaagcagtatgtggatggagatcagtgtgagtccaacccctgtctgaatggagggtcttgcaaagatgatatcaactcctatgagtgctggtgtccttttggatttgaaggcaaaaattgtgagctggatgccacctgtaacatcaagaatggcaggtgtgagcagttctgtaaaaactctgctgataataaggtggtctgcagctgtacagaaggctataggctggctgagaaccagaagagctgtgaaccagctgtgcccttcccttgtgggagggtgtctgtcagccagacctctaagctgaccagagctgagactgtgttcccagatgtggattatgtcaactccacagaggctgaaaccatcctggacaacatcacccagtctacccagtccttcaatgactttaccagagtggtgggaggagaggatgccaaaccaggccagttcccctggcaggtggtcctgaatgggaaggtggatgctttttgtgggggatccattgtgaatgagaaatggattgtcacagctgctcactgtgtggagacaggggtcaagatcactgtggtggctggagagcacaacattgaggaaacagaacatactgagcagaagaggaatgtgatcagaatcatccctcaccataactacaatgctgctatcaacaagtactcccacgacatcgccctgctggagctggacaagcccctgaccctgaacagctacgtgacccccatctgcatcgccaacagggagtacaccaacatcttcctgaagttcggatccggatacgtgagcggatggggacgcgtgttcaaccgcggccggtccgccagcatcctgcagtacctgagagtgccactggtggacagagctacctgcctgcggtccaccaagttcaccatctacaacaacatgttctgcgctggataccacgagggagggaaggactcctgccagggggacagcggaggaccacacgtgaccgaggtggagggcacctccttcctgaccggcatcatcagctggggggaggagtgcgccatgaagggcaagtacgggatctacaccaaggtgagcagatacgtgaactggatcaaggagaagaccaagctgacctga

SEQ ID NO: 9 - DNA, 1386 bp, Artificial Sequence
  Synthetic hfIX-96ge2appro - hfIX with An96 Gla, EGF2, Activation Peptide, and Protease Domains
atgcagagggtcaatatgatcatggctgaatctcctgggctgatcaccatttgcctgctgggatacctgctgtctgctgagtgtacagtgttcctggaccatgagaatgccaataagatcctgaacaggcccaaaagatacaactccggcaagctggaggagttcgtgagggggaacctggagagagagtgcatcgaggagaagtgcagcttcgaggaggccagggaggtgttcgagaacaccgagaagaccaccgagttctggaagcagtacgtggatggagatcagtgtgagtccaacccctgtctgaatggagggtcttgcaaagatgatatcaactcctatgagtgctggtgtccttttggatttgaaggcaaaaattgtgagctggacgccacctgcagcatcaagaacggcagatgcaagcagttctgcaagaagtccgccgacaacaaggtggtgtgcagctgcaccgagggatacagactggctgaggaccagaagtcctgcgagccagctgtgcccttcccttgtgggagggtgtctgtcagccagacctctaagctgaccagagccgaaaccatcttctccaacatggactacgagaacagcaccgaggccgaaaccatcctggacaacgtgacccagtccacccagagcttcaacgacttcacccgggtggtgggaggagagaacgctaagccaggacagttcccatggcaggtgctgctgaacgggaagatcgacgccttctgcggcgggtccatcatcaacgagaagtgggtggtgaccgctgctcactgcatcgagccaggagtgaagatcaccgtggtggctggggagcacaacatcgagaagaccgagcccaccgagcagaagcgcaacgtgatccgcgtgatcccccaccacaactacaacgccaccatcaacaagtactcccacgacatcgccctgctggagctggacaagcccctgaccctgaacagctacgtgacccccatctgcatcgccaacagggagtacaccaacatcttcctgaagttcggatccggatacgtgagcggatggggacgcgtgttcaaccgcggccggtccgccagcatcctgcagtacctgagagtgccactggtggacagagctacctgcctgcggtccaccaagttcaccatctacaacaacatgttctgcgctggataccacgagggagggaaggactcctgccagggggacagcggaggaccacacgtgaccgaggtggagggcacctccttcctgaccggcatcatcagctggggggaggagtgcgccatgaagggcaagtacgggatctacaccaaggtgagcagatacgtgaactggatcaaggagaagaccaagctgacctga

SEQ ID NO: 10 - DNA, 1386 bp, Artificial Sequence
  Synthetic hfIX-96ge2pro - hfIX with An96 Gla, EGF2, and Protease Domains
atgcagagggtcaatatgatcatggctgaatctcctgggctgatcaccatttgcctgctgggatacctgctgtctgctgagtgtacagtgttcctggaccatgagaatgccaataagatcctgaacaggcccaaaagatacaactccggcaagctggaggagttcgtgagggggaacctggagagagagtgcatcgaggagaagtgcagcttcgaggaggccagggaggtgttcgagaacaccgagaagaccaccgagttctggaagcagtacgtggatggagatcagtgtgagtccaacccctgtctgaatggagggtcttgcaaagatgatatcaactcctatgagtgctggtgtccttttggatttgaaggcaaaaattgtgagctggacgccacctgcagcatcaagaacggcagatgcaagcagttctgcaagaagtccgccgacaacaaggtggtgtgcagctgcaccgagggatacagactggctgaggaccagaagtcctgcgagccagctgtgcccttcccttgtgggagggtgtctgtcagccagacctctaagctgaccagagctgagactgtgttcccagatgtggattatgtcaactccacagaggctgaaaccatcctggacaacatcacccagtctacccagtccttcaatgactttaccagagtggtgggaggagagaacgctaagccaggacagttcccatggcaggtgctgctgaacgggaagatcgacgccttctgcggcgggtccatcatcaacgagaagtgggtggtgaccgctgctcactgcatcgagccaggagtgaagatcaccgtggtggctggggagcacaacatcgagaagaccgagcccaccgagcagaagcgcaacgtgatccgcgtgatcccccaccacaactacaacgccaccatcaacaagtactcccacgacatcgccctgctggagctggacaagcccctgaccctgaacagctacgtgacccccatctgcatcgccaacagggagtacaccaacatcttcctgaagttcggatccggatacgtgagcggatggggacgcgtgttcaaccgcggccggtccgccagcatcctgcagtacctgagagtgccactggtggacagagctacctgcctgcggtccaccaagttcaccatctacaacaacatgttctgcgctggataccacgagggagggaaggactcctgccagggggacagcggaggaccacacgtgaccgaggtggagggcacctccttcctgaccggcatcatcagctggggggaggagtgcgccatgaagggcaagtacgggatctacaccaaggtgagcagatacgtgaactggatcaaggagaagaccaagctgacctga

SEQ ID NO: 11 - DNA, 1386 bp, Artificial Sequence
  Synthetic hfIX-96ge2pro2  - hfIX containing An96 Gla, EGF2, and Pro2 Domains
atgcagagggtcaatatgatcatggctgaatctcctgggctgatcaccatttgcctgctgggatacctgctgtctgctgagtgtacagtgttcctggaccatgagaatgccaataagatcctgaacaggcccaaaagatacaactccggcaagctggaggagttcgtgagggggaacctggagagagagtgcatcgaggagaagtgcagcttcgaggaggccagggaggtgttcgagaacaccgagaagaccaccgagttctggaagcagtacgtggatggagatcagtgtgagtccaacccctgtctgaatggagggtcttgcaaagatgatatcaactcctatgagtgctggtgtccttttggatttgaaggcaaaaattgtgagctggacgccacctgcagcatcaagaacggcagatgcaagcagttctgcaagaagtccgccgacaacaaggtggtgtgcagctgcaccgagggatacagactggctgaggaccagaagtcctgcgagccagctgtgcccttcccttgtgggagggtgtctgtcagccagacctctaagctgaccagagctgagactgtgttcccagatgtggattatgtcaactccacagaggctgaaaccatcctggacaacatcacccagtctacccagtccttcaatgactttaccagagtggtgggaggagaggatgccaaaccaggccagttcccctggcaggtggtcctgaatgggaaggtggatgctttttgtgggggatccattgtgaatgagaaatggattgtcacagctgctcactgtgtggagacaggggtcaagatcactgtggtggctggagagcacaacattgaggaaacagaacatactgagcagaagaggaatgtgatcagaatcatccctcaccataactacaatgctgctatcaacaagtactcccacgacatcgccctgctggagctggacaagcccctgaccctgaacagctacgtgacccccatctgcatcgccaacagggagtacaccaacatcttcctgaagttcggatccggatacgtgagcggatggggacgcgtgttcaaccgcggccggtccgccagcatcctgcagtacctgagagtgccactggtggacagagctacctgcctgcggtccaccaagttcaccatctacaacaacatgttctgcgctggataccacgagggagggaaggactcctgccagggggacagcggaggaccacacgtgaccgaggtggagggcacctccttcctgaccggcatcatcagctggggggaggagtgcgccatgaagggcaagtacgggatctacaccaaggtgagcagatacgtgaactggatcaaggagaagaccaagctgacctga

SEQ ID NO: 12 - DNA, 1386 bp, Artificial Sequence
  Synthetic hfIX-96gpro - hfIX with An96 Gla and Protease Domains
atgcagagggtcaatatgatcatggctgaatctcctgggctgatcaccatttgcctgctgggatacctgctgtctgctgagtgtacagtgttcctggaccatgagaatgccaataagatcctgaacaggcccaaaagatacaactccggcaagctggaggagttcgtgagggggaacctggagagagagtgcatcgaggagaagtgcagcttcgaggaggccagggaggtgttcgagaacaccgagaagaccaccgagttctggaagcagtacgtggatggagatcagtgtgagtccaacccctgtctgaatggagggtcttgcaaagatgatatcaactcctatgagtgctggtgtccttttggatttgaaggcaaaaattgtgagctggatgtgacctgtaacatcaagaatggcaggtgtgagcagttctgtaaaaactctgctgataataaggtggtctgcagctgtacagaaggctataggctggctgagaaccagaagagctgtgaaccagctgtgcccttcccttgtgggagggtgtctgtcagccagacctctaagctgaccagagctgagactgtgttcccagatgtggattatgtcaactccacagaggctgaaaccatcctggacaacatcacccagtctacccagtccttcaatgactttaccagagtggtgggaggagagaacgctaagccaggacagttcccatggcaggtgctgctgaacgggaagatcgacgccttctgcggcgggtccatcatcaacgagaagtgggtggtgaccgctgctcactgcatcgagccaggagtgaagatcaccgtggtggctggggagcacaacatcgagaagaccgagcccaccgagcagaagcgcaacgtgatccgcgtgatcccccaccacaactacaacgccaccatcaacaagtactcccacgacatcgccctgctggagctggacaagcccctgaccctgaacagctacgtgacccccatctgcatcgccaacagggagtacaccaacatcttcctgaagttcggatccggatacgtgagcggatggggacgcgtgttcaaccgcggccggtccgccagcatcctgcagtacctgagagtgccactggtggacagagctacctgcctgcggtccaccaagttcaccatctacaacaacatgttctgcgctggataccacgagggagggaaggactcctgccagggggacagcggaggaccacacgtgaccgaggtggagggcacctccttcctgaccggcatcatcagctggggggaggagtgcgccatgaagggcaagtacgggatctacaccaaggtgagcagatacgtgaactggatcaaggagaagaccaagctgacctga

SEQ ID NO: 13 - DNA, 1386 bp, Artificial Sequence
  Synthetic hfIX-96pro - hfIX with An96 Protease Domain
atgcagagggtcaatatgatcatggctgaatctcctgggctgatcaccatttgcctgctgggatacctgctgtctgctgagtgtacagtgttcctggaccatgagaatgccaataagatcctgaacaggcccaaaagatacaattctggaaagctggaggaatttgtgcagggcaacctggagagggaatgcatggaggaaaagtgtagctttgaggaagctagggaggtgtttgaaaacacagagaggaccacagaattctggaagcagtatgtggatggagatcagtgtgagtccaacccctgtctgaatggagggtcttgcaaagatgatatcaactcctatgagtgctggtgtccttttggatttgaaggcaaaaattgtgagctggatgtgacctgtaacatcaagaatggcaggtgtgagcagttctgtaaaaactctgctgataataaggtggtctgcagctgtacagaaggctataggctggctgagaaccagaagagctgtgaaccagctgtgcccttcccttgtgggagggtgtctgtcagccagacctctaagctgaccagagctgagactgtgttcccagatgtggattatgtcaactccacagaggctgaaaccatcctggacaacatcacccagtctacccagtccttcaatgactttaccagagtggtgggaggagagaacgctaagccaggacagttcccatggcaggtgctgctgaacgggaagatcgacgccttctgcggcgggtccatcatcaacgagaagtgggtggtgaccgctgctcactgcatcgagccaggagtgaagatcaccgtggtggctggggagcacaacatcgagaagaccgagcccaccgagcagaagcgcaacgtgatccgcgtgatcccccaccacaactacaacgccaccatcaacaagtactcccacgacatcgccctgctggagctggacaagcccctgaccctgaacagctacgtgacccccatctgcatcgccaacagggagtacaccaacatcttcctgaagttcggatccggatacgtgagcggatggggacgcgtgttcaaccgcggccggtccgccagcatcctgcagtacctgagagtgccactggtggacagagctacctgcctgcggtccaccaagttcaccatctacaacaacatgttctgcgctggataccacgagggagggaaggactcctgccagggggacagcggaggaccacacgtgaccgaggtggagggcacctccttcctgaccggcatcatcagctggggggaggagtgcgccatgaagggcaagtacgggatctacaccaaggtgagcagatacgtgaactggatcaaggagaagaccaagctgacctga

SEQ ID NO: 14 - DNA, 1386 bp, Artificial Sequence
  Synthetic hfIX-96pro2 - hfIX with An96 second half of Protease Domain
atgcagagggtcaatatgatcatggctgaatctcctgggctgatcaccatttgcctgctgggatacctgctgtctgctgagtgtacagtgttcctggaccatgagaatgccaataagatcctgaacaggcccaaaagatacaattctggaaagctggaggaatttgtgcagggcaacctggagagggaatgcatggaggaaaagtgtagctttgaggaagctagggaggtgtttgaaaacacagagaggaccacagaattctggaagcagtatgtggatggagatcagtgtgagtccaacccctgtctgaatggagggtcttgcaaagatgatatcaactcctatgagtgctggtgtccttttggatttgaaggcaaaaattgtgagctggatgtgacctgtaacatcaagaatggcaggtgtgagcagttctgtaaaaactctgctgataataaggtggtctgcagctgtacagaaggctataggctggctgagaaccagaagagctgtgaaccagctgtgcccttcccttgtgggagggtgtctgtcagccagacctctaagctgaccagagctgagactgtgttcccagatgtggattatgtcaactccacagaggctgaaaccatcctggacaacatcacccagtctacccagtccttcaatgactttaccagagtggtgggaggagaggatgccaaaccaggccagttcccctggcaggtggtcctgaatgggaaggtggatgctttttgtgggggatccattgtgaatgagaaatggattgtcacagctgctcactgtgtggagacaggggtcaagatcactgtggtggctggagagcacaacattgaggaaacagaacatactgagcagaagaggaatgtgatcagaatcatccctcaccataactacaatgctgctatcaacaagtactcccacgacatcgccctgctggagctggacaagcccctgaccctgaacagctacgtgacccccatctgcatcgccaacagggagtacaccaacatcttcctgaagttcggatccggatacgtgagcggatggggacgcgtgttcaaccgcggccggtccgccagcatcctgcagtacctgagagtgccactggtggacagagctacctgcctgcggtccaccaagttcaccatctacaacaacatgttctgcgctggataccacgagggagggaaggactcctgccagggggacagcggaggaccacacgtgaccgaggtggagggcacctccttcctgaccggcatcatcagctggggggaggagtgcgccatgaagggcaagtacgggatctacaccaaggtgagcagatacgtgaactggatcaaggagaagaccaagctgacctga

SEQ ID NO: 15 - protein, 415 aa, Homo sapiens
  SITE (1)..(415): Human fIX
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFNKGRSASVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 16 - protein, 462 aa, Artificial Sequence
  Synthetic An96 fIX Padua
MQCLNMIMAESPGLITICLLGYLLSAECTVFLDHENATKILNRPKRYNSGKLEEFVRGNLERECIEEKCSFEEAREVFENTEKTTEFWKQYVDGDQCESNPCLNGGSCKDDINSYECWCRFGFEGKNCELDATCSIKNGRCKQFCKKSADNKVVCSCTEGYRLAEDQKSCEPAVPFPCGRVSVSHTSKKLTRAETIFSNMDYENSTEAETILDNVTQSTQSFNDFTRVVGGENAKPGQFPWQVLLNGKIDAFCGGSIINEKWVVTAAHCIEPGVKITVVAGEHNIEKTEPTEQKRNVIRVIPHHNYNATINKYSHDIALLELDKPLTLNSYVTPICIANREYTNIFLKFGSGYVSGWGRVFNRGRSASILQYLRVPLVDRATCLLSTKFTIYNNMFCAGYHEGGKDSCQGDSGGPHVTEVEGTSFLTGIISWGEECAMKGKYGIYTKVSRYVNWIKEKTKLT

SEQ ID NO: 17 - DNA, 1389 bp, Artificial Sequence
  Synthetic An96 fIX Padua
atgcagtgcctgaacatgatcatggccgagtcccccggcctgatcaccatctgcctgctggggtacctgctgagcgccgagtgcaccgtgttcctggaccacgagaacgccaccaagatcctgaacaggcccaagagatacaactccggcaagctggaggagttcgtgagggggaacctggagagagagtgcatcgaggagaagtgcagcttcgaggaggccagggaggtgttcgagaacaccgagaagaccaccgagttctggaagcagtacgtggacggcgaccagtgcgagtccaacccctgcctgaacggcgggtcctgcaaggacgacatcaacagctacgagtgctggtgcaggttcggcttcgaggggaagaactgcgagctggacgccacctgcagcatcaagaacggcagatgcaagcagttctgcaagaagtccgccgacaacaaggtggtgtgcagctgcaccgagggatacagactggctgaggaccagaagtcctgcgagccagctgtgccattcccatgcgggagggtgtccgtgagccacaccagcaagaagctgaccagagccgaaaccatcttctccaacatggactacgagaacagcaccgaggccgaaaccatcctggacaacgtgacccagtccacccagagcttcaacgacttcacccgggtggtgggaggagagaacgctaagccaggacagttcccatggcaggtgctgctgaacgggaagatcgacgccttctgcggcgggtccatcatcaacgagaagtgggtggtgaccgctgctcactgcatcgagccaggagtgaagatcaccgtggtggctggggagcacaacatcgagaagaccgagcccaccgagcagaagcgcaacgtgatccgcgtgatcccccaccacaactacaacgccaccatcaacaagtactcccacgacatcgccctgctggagctggacaagcccctgaccctgaacagctacgtgacccccatctgcatcgccaacagggagtacaccaacatcttcctgaagttcggatccggatacgtgagcggatggggacgcgtgttcaaccgcggccggtccgccagcatcctgcagtacctgagagtgccactggtggacagagctacctgcctgctgtccaccaagttcaccatctacaacaacatgttctgcgctggataccacgagggagggaaggactcctgccagggggacagcggaggaccacacgtgaccgaggtggagggcacctccttcctgaccggcatcatcagctggggggaggagtgcgccatgaagggcaagtacgggatctacaccaaggtgagcagatacgtgaactggatcaaggagaagaccaagctgacctga

SEQ ID NO: 18 - protein, 461 aa, Artificial Sequence
  Synthetic An97 fIX Padua
MQCLNMIMAESPGLITICLLGYLLSAECTVFLDHENANKILNRPKRYNSGKLEEFVRGNLERECIEEKCSFEEAREVFENTEKTTEFWKQYVDGDQCESNPCLNGGSCKDDINSYECWCRFGFEGKNCELDATCSIKNGRCKQFCKKSADNKVVCSCTEGYRLAEDQKSCEPAVPFPCGRVSVSHTSKLTRAETIFSNMDYENSTEAETILDNVTQSTQSFNDFTRVVGGENAKPGQFPWQVLLNGKIDAFCGGSIINEKWVVTAAHCIEPGVKITVVAGEHNIEKTEPTEQKRNVIRVIPHHNYNATINKYSHDIALLELDKPLTLNSYVTPICIADREYTNIFLKFGSGYVSGWGRVFNRGRSASILQYLRVPLVDRATCLLSTKFTIYNNMFCAGYHEGGKDSCQGDSGGPHVTEVEGTSFLTGIISWGEECAMKGKYGIYTKVSRYVNWIKEKTKLT

SEQ ID NO: 19 - protein, 462 aa, Artificial Sequence
  Synthetic An63 fIX
MQCLNMIMAESPGLITICLLGYLLSAECTVFLDHENATKILNRPKRYNSGKLEEFVQGNLERECIEEKCSFEEAREVFENTEKTTEFWKQYVDGDQCESNPCLNGGMCKDDINSYECWCQVGFEGKNCELDATCSIKNGRCKQFCKKGADNKVVCSCTTGYRLAEDQKSCEPAVPFPCGRVSVSHTSKKLTRAETIFSNMDYENSTEAETILDNVTQSTQSFNDFTRVVGGENAKPGQFPWQVLLNGKIDAFCGGSIINEKWVVTAAHCIEPGVKITVVAGEHNTEETEHTEQKRNVIRVIPHHSYNATINKYSHDIALLELDKPLTLNSYVTPICIANREYTNIFLKFGSGYVSGWGKVFNRGRSASILQYLKVPLVDRATCLRSTKFTIYNNMFCAGYHEGGKDSCQGDSGGPHVTEVEGTSFLTGIISWGEECAMKGKYGIYTKVSRYVNWIKEKTKLT

SEQ ID NO: 20 - protein, 462 aa, Artificial Sequence
  Synthetic An65 fIX
MQCLNMIMAESPGLITICLLGYLLSAECTVFLDHENATKILNRPKRYNSGKLEEFVQGNLERECIEEKCSFEEAREVFENTEKTTEFWKQYVDGDQCESNPCLNGGLCKDDINSYECWCQVGFEGKNCELDATCSIKNGRCKQFCKKGADNKVVCSCTTGYRLAEDQKSCEPAVPFPCGRVSVSHTSTKLTRAETIFSNMDYENSTEAETILDNVTQSTQSFNDFTRVVGGENAKPGQFPWQVLLNGKIDAFCGGSIINEKWVVTAAHCIEPGVKITVVAGEHNTEETEHTEQKRNVIRVIPHHSYNATINKYSHDIALLELDKPLTLNSYVTPICIADREYTNIFLKFGSGYVSGWGKVFNRGRSASILQYLKVPLVDRATCLRSTKFTIYNNMFCAGFHEGGKDSCQGDSGGPHVTEVEGTSFLTGIISWGEECAMKGKYGIYTKVSRYVNWIKEKTKLT

SEQ ID NO: 21 - protein, 462 aa, Artificial Sequence
  Synthetic An70 fIX
MQCLNMIMAESPGLVTICLLGYLLSAECTVFLDRENATKILNRPKRYNSGKLEEFVRGNLERECIEEKCSFEEAREVFENTEKTTEFWKQYVDGDQCESNPCLNGGLCKDDINSYECWCQVGFEGKNCELDATCSIKNGRCKQFCKKGADNKVVCSCTTGYRLAEDQKSCEPAVPFPCGRVSVSHTSTKLTRAETIFSNMDYENSTEAEIILDNVTQSNQSFNDFTRIVGGENAKPGQFPWQVLLNGKIDAFCGGSIINEKWVVTAAHCIEPGVKITVVAGEHNTEETEPTEQKRNVIRAIPHHSYNATVNKYSHDIALLELDEPLTLNSYVTPICIADREYTNIFLKFGSGYVSGWGKVFNRGRSASILQYLKVPLVDRATCLRSTKFTIYNNMFCAGFHEGGKDSCQGDSGGPHVTEVEGTSFLTGIISWGEECAVKGKYGIYTKVSRYVNWIKEKTKLT

SEQ ID NO: 22 - protein, 462 aa, Artificial Sequence
  Synthetic An84 fIX
MQCLNMIMAESPGLITICLLGYLLSAECTVFLDHENATKILNRPKRYNSGKLEEFVQGNLERECIEEKCSFEEAREVFENTEKTTEFWKQYVDGDQCESNPCLNGGSCKDDINSYECWCQVGFEGKNCELDATCSIKNGRCKQFCKKGADNKVVCSCTEGYRLAEDQKSCEPAVPFPCGRVSVSHTSKKLTRAETIFSNMDYENSTEAETILDNVTQSTQSFNDFTRVVGGENAKPGQFPWQVLLNGKIDAFCGGSIINEKWVVTAAHCIEPGVKITVVAGEHNIEETEHTEQKRNVIRVIPHHNYNATINKYSHDIALLELDKPLTLNSYVTPICIANREYTNIFLKFGSGYVSGWGRVFNRGRSASILQYLRVPLVDRATCLRSTKFTIYNNMFCAGYHEGGKDSCQGDSGGPHVTEVEGTSFLTGIISWGEECAMKGKYGIYTKVSRYVNWIKEKTKLT

SEQ ID NO: 23 - protein, 462 aa, Artificial Sequence
  Synthetic An88 fIX
MQHLNTIMAESPGLITIFLLGYLLSAECAVFLDRENATKILTRPKRYNSGKLEEFVQGNLERECIEERCSFEEAREVFENTEKTTEFWKQYVDGDQCESNPCLNGGKCKDDINSYECWCQVGFEGRNCELDATCNIKNGRCKQFCKNGADNKVICSCTEGYQLAEDQKSCEPAVPFPCGRVSVSYSSKKLTRAETIFSNMDYENSTEAETILDNVTENSESLNDFTRVVGGENAKPGQIPWQVILNGEIEAFCGGAIINEKWVVTAAHCLKPGDKIEVVAGEYNIDEKEDTEQRRNVIRTIPHHHYNATINKYSHDIALLELDKPLILNSYVTPICVANREYTNIFLKFGSGYVSGWGKVFNKGRQASILQYLRVPLVDRATCLRSTTFTIYNNMFCAGYREGGKDSCEGDSGGPHVTEVEGTSFLTGIISWGEECAMKGKYGIYTKVSRYVNWIKEKTKLT

SEQ ID NO: 24 - protein, 462 aa, Artificial Sequence; identical to SEQ ID NO: 22
  Synthetic An95 fIX

SEQ ID NO: 25 - protein, 461 aa, Artificial Sequence
  Synthetic An97 fIX
MQCLNMIMAESPGLITICLLGYLLSAECTVFLDHENANKILNRPKRYNSGKLEEFVRGNLERECIEEKCSFEEAREVFENTEKTTEFWKQYVDGDQCESNPCLNGGSCKDDINSYECWCRFGFEGKNCELDATCSIKNGRCKQFCKKSADNKVVCSCTEGYRLAEDQKSCEPAVPFPCGRVSVSHTSKLTRAETIFSNMDYENSTEAETILDNVTQSTQSFNDFTRVVGGENAKPGQFPWQVLLNGKIDAFCGGSIINEKWVVTAAHCIEPGVKITVVAGEHNIEKTEPTEQKRNVIRVIPHHNYNATINKYSHDIALLELDKPLTLNSYVTPICIADREYTNIFLKFGSGYVSGWGRVFNRGRSASILQYLRVPLVDRATCLRSTKFTIYNNMFCAGYHEGGKDSCQGDSGGPHVTEVEGTSFLTGIISWGEECAMKGKYGIYTKVSRYVNWIKEKTKLT

SEQ ID NO: 26 - protein, 461 aa, Artificial Sequence
  Synthetic An102 fIX
MQRVNMIMAESPGLITICLLGYLLSAECTVFLDHENANKILNRPKRYNSGKLEEFVQGNLERECMEEKCSFEEAREVFENTEKTTEFWKQYVDGDQCESNPCLNGGSCKDDINSYECWCPFGFEGKNCELDVTCNIKNGRCKQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWVVTAAHCIETGVKITVVAGEHNIEETEHTEQKRNVIRIIPHHNYNATINKYNHDIALLELDEPLVLNSYVTPICIADKEYTNIFLKFGSGYVSGWGRVFNKGRSASVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 27 - DNA, 1386 bp, Artificial Sequence
  Synthetic An97 fIX Padua
atgcagtgcctgaacatgatcatggccgagtcccccggcctgatcaccatctgcctgctggggtacctgctgagcgccgagtgcaccgtgttcctggaccacgagaacgccaacaagatcctgaacaggcccaagagatacaactccggcaagctggaggagttcgtgagggggaacctggagagagagtgcatcgaggagaagtgcagcttcgaggaggccagggaggtgttcgagaacaccgagaagaccaccgagttctggaagcagtacgtggacggcgaccagtgcgagtccaacccctgcctgaacggcgggtcctgcaaggacgacatcaacagctacgagtgctggtgcaggttcggcttcgaggggaagaactgcgagctggacgccacctgcagcatcaagaacggcagatgcaagcagttctgcaagaagtccgccgacaacaaggtggtgtgcagctgcaccgagggatacagactggctgaggaccagaagtcctgcgagccagctgtgccattcccatgcgggagggtgtccgtgagccacaccagcaagctgaccagagccgaaaccatcttctccaacatggactacgagaacagcaccgaggccgaaaccatcctggacaacgtgacccagtccacccagagcttcaacgacttcacccgggtggtgggaggagagaacgctaagccaggacagttcccatggcaggtgctgctgaacgggaagatcgacgccttctgcggcgggtccatcatcaacgagaagtgggtggtgaccgctgctcactgcatcgagccaggagtgaagatcaccgtggtggctggggagcacaacatcgagaagaccgagcccaccgagcagaagcgcaacgtgatccgcgtgatcccccaccacaactacaacgccaccatcaacaagtactcccacgacatcgccctgctggagctggacaagcccctgaccctgaacagctacgtgacccccatctgcatcgccgacagggagtacaccaacatcttcctgaagttcggatccggatacgtgagcggatggggacgcgtgttcaaccgcggccggtccgccagcatcctgcagtacctgagagtgccactggtggacagagctacctgcctgctgtccaccaagttcaccatctacaacaacatgttctgcgctggataccacgagggagggaaggactcctgccagggggacagcggaggaccacacgtgaccgaggtggagggcacctccttcctgaccggcatcatcagctggggggaggagtgcgccatgaagggcaagtacgggatctacaccaaggtgagcagatacgtgaactggatcaaggagaagaccaagctgacctga

SEQ ID NO: 28 - DNA, 1386 bp, Artificial Sequence
  Synthetic FIX Optimized Sequence Alpha 1-No Padua
atgcagagggtgaacatgatcatggctgagtcccctggcctcatcaccatctgcctcctgggctacctcctgagtgctgagtgcacagtgttcctggaccatgagaatgccaacaagatcctcaacaggcccaagaggtacaactctggcaagctggaggagtttgtgcagggcaacctggagagggagtgcatggaggagaagtgctcctttgaggaggccagggaggtctttgagaacacagagaggaccacagagttctggaagcagtatgtggatggggaccagtgtgagtccaacccctgcctcaatgggggctcctgcaaggatgacatcaactcctatgagtgctggtgcccctttggctttgagggcaagaactgtgagctggatgccacctgcaacatcaagaatgggaggtgtgagcagttctgcaagaactctgctgacaacaaggtggtgtgctcctgcactgagggctacaggctggctgagaaccagaagtcctgtgagcctgctgtgcccttcccctgtgggagggtgagtgtgtcccagacctccaagctcaccagggctgagactgtgttccctgatgtggactatgtgaactccactgaggctgagaccatcctggacaacatcacccagagcacccagagcttcaatgacttcaccagggtggtggggggggaggatgccaagcctggccagttcccctggcaggtggtgctcaatggcaaggtggatgccttctgtgggggctccattgtgaatgagaagtggattgtgactgctgcccactgtgtggagacaggggtgaagatcacagtggtggctggggagcacaacattgaggagacagagcacacagagcagaagaggaatgtgatcaggatcatcccccaccacaactacaatgctgccatcaacaagtacaaccatgacattgccctcctggagctggatgagcccctggtgctcaactcctatgtgacccccatctgcattgccaacaaggagtacaccaacatcttcctcaagtttggctctggctatgtgagtggctgggggagggtgttccacaaggggaggagtgcctctgtgctccagtacctgagggtgcccctggtggacagggccacctgcctgaggagcaccaagttcaccatctacaacaacatgttctgtgctggcttccatgagggggggagggactcctgccagggggactctgggggcccccatgtgactgaggtggagggcacctccttcctcactggcatcatctcctggggggaggagtgtgccatgaagggcaagtatggcatctacaccaaggtgagcaggtatgtgaactggatcaaggagaagaccaagttgacctga

SEQ ID NO: 29 - DNA, 1386 bp, Artificial Sequence
  Synthetic FIX Optimized Sequence Alpha 2-No Padua
atgcagagggtgaacatgatcatggctgagtcccctggcctgatcaccatctgcctcctgggctacctgctgagtgctgagtgcacagtgttcctggaccatgagaatgccaacaagatcctgaacaggcccaagaggtacaactctgggaagctggaggagtttgtccaggggaacctggagagggagtgcatggaggagaagtgcagctttgaggaggccagggaggtgtttgagaacacagagaggaccacagagttctggaagcagtatgtggatggagaccagtgtgagtccaacccctgcctgaatgggggcagctgcaaggatgacatcaacagctatgagtgctggtgcccctttggctttgaggggaagaactgtgagctggatgccacctgcaacatcaagaatggcaggtgtgagcagttctgcaagaactctgctgataacaaggtggtgtgcagctgcacagagggctataggctggctgagaaccagaagagctgtgagcctgctgtgcccttcccctgtggcagggtgagtgtgtcccagacctccaagctgaccagggctgagacagtcttccctgatgtggactatgtcaacagcacagaggctgagaccatcctggacaacatcacccagtccacccagagcttcaatgacttcaccagggtggtggggggggaggatgccaagcctgggcagttcccctggcaggtggtgctgaatggcaaggtggatgccttctgtggggggagcattgtcaatgagaagtggattgtcacagctgcccactgtgtggagacaggggtgaagatcacagtggtggctggagagcacaacattgaggagacagagcacacagagcagaagaggaatgtcatcaggatcatcccccaccacaactacaatgctgccatcaacaagtacaaccatgacattgccctgctggagctggatgagcccctggtgctgaacagctatgtcacccccatctgcattgccaacaaggagtacaccaacatcttcctgaagtttggctctggctatgtctctggctggggcagggtgttccacaagggcagatctgcctctgtcctgcagtacctgagggtccccctggtggatagggccacctgcctgaggagcaccaagttcaccatctacaacaacatgttctgtgctgggttccatgagggggggagggactcctgccagggggatagtggagggccccatgtgacagaggtggagggcacctccttcctcacaggcatcatctcctggggggaggagtgtgccatgaagggcaagtatggcatctacaccaaggtcagcaggtatgtcaactggatcaaggagaagaccaagctcacctga

SEQ ID NO: 30 - DNA, 1386 bp, Artificial Sequence
  Synthetic FIX Optimized Sequence Alpha 3-No Padua
atgcagagggtcaacatgatcatggctgagagccctgggctcatcaccatctgcctgctgggctacctgctgtctgctgagtgcactgtgttcctggaccatgagaatgccaacaagatcctgaacagacccaagaggtacaactcaggcaagctggaggagtttgtgcagggcaacctggagagggagtgcatggaggagaagtgctcctttgaggaggccagagaggtctttgagaacacagagaggaccactgagttctggaagcagtatgtggatggggaccagtgtgagtccaacccctgcctgaatggaggcagctgcaaggatgacatcaacagctatgagtgctggtgcccctttggctttgagggcaagaactgtgagctggatgccacctgcaacatcaagaatggcagatgtgagcagttctgcaagaactcagctgacaacaaggtggtgtgcagctgcacagagggctacagactggctgagaaccagaagagctgtgagcctgctgtgcccttcccctgtggcagggtctctgtcagccagacctccaagctcaccagagctgagactgtgttccctgatgtggactatgtgaacagcacagaggctgagaccatcctggacaacatcacccagagcacccagtccttcaatgacttcaccagagtggtggggggggaggatgccaagccagggcagttcccctggcaggtggtgctgaatggcaaggtggatgccttctgtgggggctccattgtgaatgagaagtggattgtgacagctgcccactgtgtggagacaggggtcaagatcacagtggtggctggagagcacaacatagaggagacagagcacacagagcagaagaggaatgtcatcaggatcatcccccaccacaactacaatgcagccatcaacaagtacaaccatgacatagccctgctggagctggatgagcccctggtgctgaacagctatgtgacccccatctgcatagccaacaaggagtacaccaacatcttcctgaagtttggctctggctatgtgtctggctggggcagggtcttccacaagggcagatctgccagtgtgctgcagtacctgagagtgcccctggtggacagggccacctgcctgaggagcactaagttcaccatctacaacaacatgttctgtgctggcttccatgaggggggcagagacagctgccagggggactctgggggcccccatgtgacagaggtggagggcaccagcttcctgacaggcatcatcagctggggggaggagtgtgccatgaagggcaagtatggcatctacaccaaggtcagcagatatgtgaactggatcaaggagaagaccaagctgacctga

SEQ ID NO: 31 - DNA, 1386 bp, Artificial Sequence
  Synthetic FIX Optimized Sequence Alpha 4-No Padua
atgcagagggtgaacatgatcatggctgagtcccctggcctgatcaccatctgcctgctgggctacctgctgtctgctgagtgcacagtgttcctggaccatgagaatgccaacaagatcctgaacaggcccaagaggtacaacagtggcaagctggaggagtttgtgcagggcaacctggagagggagtgcatggaggagaagtgcagctttgaggaggccagggaggtgtttgagaacacagagaggaccacagagttctggaagcagtatgtggatggggaccagtgtgagtccaacccctgcctgaatgggggcagctgcaaggatgacatcaactcctatgagtgctggtgcccctttggctttgagggcaagaactgtgagctggatgccacctgcaacatcaagaatggcaggtgtgagcagttctgcaagaacagtgctgacaacaaggtggtgtgctcctgcacagagggctacaggctggctgagaaccagaagtcctgtgagcctgctgtgcccttcccctgtggcagggtgtctgtgtcccagacctccaagctgaccagggctgagacagtgttccctgatgtggactatgtgaactccacagaggctgagaccatcctggacaacatcacccagtccacccagagcttcaatgacttcaccagggtggtggggggggaggatgccaagcctggccagttcccctggcaggtggtgctgaatggcaaggtggatgccttctgtgggggctccatagtgaatgagaagtggattgtgactgctgcccactgtgtggagacaggggtgaagatcacagtggtggctggggagcacaacattgaggagacagagcacacagagcagaagaggaatgtgatcaggatcatcccccaccacaactacaatgctgccatcaacaagtacaaccatgacattgccctgctggagctggatgagcccctggtgctgaactcctatgtgacccccatctgcatagccaacaaggagtacaccaacatcttcctgaagtttggcagtggctatgtgtctggctggggcagggtgttccacaagggcaggtctgcctctgtgctgcagtacctgagggtgcccctggtggacagggccacctgcctgaggagcaccaagttcaccatctacaacaacatgttctgtgctggcttccatgaggggggcagggactcctgccagggggactctgggggcccccatgtgactgaggtggagggcacctccttcctgacaggcatcatctcctggggggaggagtgtgccatgaagggcaagtatggcatctacaccaaggtgagcaggtatgtgaactggatcaaggagaagaccaagctgacctga

SEQ ID NO: 32 - DNA, 1386 bp, Artificial Sequence
  Synthetic FIX Optimized Sequence Beta 1-No Padua
atgcagagggtgaacatgatcatggctgagagccctgggctgatcaccatctgcctgctggggtacctgctgagtgctgagtgcactgtgttcctggaccatgagaatgccaacaagatcctcaacaggcccaagaggtacaacagtggcaagctggaggagtttgtgcagggcaacctggagagggagtgcatggaggagaagtgcagctttgaggaggccagggaggtgtttgagaacacagagaggaccacagagttctggaagcagtatgtggatggggaccagtgtgagtccaacccctgcctgaatgggggcagctgcaaggatgacatcaacagctatgagtgctggtgcccctttggctttgagggcaagaactgtgagctggatgccacctgcaacatcaagaatggcaggtgtgagcagttctgcaagaacagtgctgacaacaaggtggtgtgcagctgcactgagggctacaggctggctgagaaccagaagtcctgtgagcctgcagtgcccttcccctgtggcagggtgtctgtgtcccagaccagcaagctcaccagggctgagactgtgttccctgatgtggactatgtcaacagcacagaggctgagaccatcctggacaacatcacccagagcacccagagcttcaatgacttcaccagggtggtggggggggaggatgccaagccagggcagttcccctggcaggtggtgctgaatggcaaggtggatgccttctgtgggggcagcattgtcaatgagaagtggattgtcacagcagcccactgtgtggagactggggtgaagatcacagtggtggctggggagcacaacattgaggagacagagcacacagagcagaagaggaatgtcatcaggatcatcccccaccacaactacaatgctgccatcaacaagtacaaccatgacattgccctgctggagctggatgagcccctggtgctgaacagctatgtcacccccatctgcattgccaacaaggagtacaccaacatcttcctgaagtttggcagtggctatgtcagtggctggggcagggtgttccacaggggcaggtctgccagtgtgctgcagtacctgagggtgcccctggtggacagggccacctgcctgaggtccaccaagttcaccatctacaacaacatgttctgtgctggcttccatgaggggggcagggacagctgccagggggactctgggggcccccatgtcactgaggtggagggcaccagcttcctgacaggcatcatcagctggggggaggagtgtgccatgaagggcaagtatggcatctacaccaaggtcagcaggtatgtgaactggatcaaggagaagaccaagctcacctga

SEQ ID NO: 33 - DNA, 1386 bp, Artificial Sequence
  Synthetic FIX Optimized Sequence Beta 2-No Padua
atgcagagggtgaacatgatcatggctgagtcccctggcctgatcaccatctgcctgctgggctacctgctgtctgctgagtgcacagtgttcctggaccatgagaatgccaacaagatcctgaacaggcccaagaggtacaactctggcaagctggaggagtttgtgcagggcaacctggagagggagtgcatggaggagaagtgctcctttgaggaggccagggaggtgtttgagaacacagagaggaccacagagttctggaagcagtatgtggatggagaccagtgtgagtccaacccttgcctgaatggaggctcctgcaaggatgacatcaactcctatgagtgctggtgcccctttggctttgagggcaagaactgtgagctggatgccacctgcaacatcaagaatggcaggtgtgagcagttctgcaagaactctgctgacaacaaggtggtgtgctcctgcacagagggctacaggctggctgagaaccagaagtcctgtgagccagctgtgcccttcccttgtggcagggtgtctgtgtcccagacctccaagctgaccagggctgagacagtgttccctgatgtggactatgtgaactccacagaggctgagaccatcctggacaacatcacccagtccacccagtccttcaatgacttcaccagggtggtgggaggtgaggatgccaagcctggccagttcccttggcaggtggtgctgaatggcaaggtggatgccttctgtggaggctccattgtgaatgagaagtggattgtgacagctgcccactgtgtggagacaggagtgaagatcacagtggtggctggtgagcacaacattgaggagacagagcacacagagcagaagaggaatgtgatcaggatcatcccccaccacaactacaatgctgccatcaacaagtacaaccatgacattgccctgctggagctggatgagcccctggtgctgaactcctatgtgacccccatctgcattgccaacaaggagtacaccaacatcttcctgaagtttggctctggctatgtgtcaggatggggcagggtgttccacaggggcaggtctgcctctgtgctgcagtacctgagggtgcccctggtggacagggccacctgcctgaggtccaccaagttcaccatctacaacaacatgttctgtgctggcttccatgagggaggcagggactcctgccagggtgactcaggaggcccccatgtgacagaggtggagggcacctccttcctgacaggcatcatcagctggggagaggagtgtgccatgaagggcaagtatggcatctacaccaaggtgtccaggtatgtgaactggatcaaggagaagaccaagctgacctga

SEQ ID NO: 34 - DNA, 1386 bp, Artificial Sequence
  Synthetic FIX Optimized Sequence Beta 3-No Padua
atgcagagagtcaacatgatcatggcagagtcacctggactgatcaccatctgcctgctgggctacctgctcagtgcagagtgcacagtcttcctggaccatgagaatgccaacaagatcctgaacagacccaagaggtacaactctggcaagctggaggagtttgtgcagggcaacctggagagagagtgcatggaggagaagtgctcctttgaggaggccagagaggtctttgagaacactgagaggaccactgagttctggaagcagtatgtggatggtgatcagtgtgagtccaacccatgcctgaatggaggctcctgcaaggatgacatcaactcctatgagtgctggtgcccatttggctttgagggcaagaactgtgagctggatgcaacatgcaacatcaagaatggcagatgtgagcagttctgcaagaacagtgcagacaacaaggtggtgtgctcctgcacagagggctacagactggctgagaaccagaagtcatgtgagcctgctgtgcccttcccctgtggcagagtgagtgtgtcccagaccagcaagctgaccagagctgagacagtcttccctgatgtggactatgtgaactccacagaggcagagaccatcctggacaacatcacacagtccacacagtccttcaatgacttcaccagagtggttggtggtgaggatgccaagcctggacagttcccctggcaggtggtgctcaatggcaaggtggatgccttctgtggaggctccattgtcaatgagaagtggattgtcacagctgcccactgtgtggagactggtgtgaagatcactgtggtggctggtgagcacaacattgaggagacagagcacactgagcagaagaggaatgtcatcaggatcatcccacaccacaactacaatgcagccatcaacaagtacaaccatgacattgccctgctggagctggatgagcccctggtgctgaacagctatgtgacccccatctgcatagccaacaaggagtacaccaacatcttcctgaagtttggctctggctatgtgtctggctggggcagagtcttccacagaggcagatctgcctctgtgctgcagtacctgagagtgcctctggtggacagagcaacctgcctgagaagcaccaagttcaccatctacaacaacatgttctgtgcaggcttccatgagggaggcagagactcctgccagggagactctggtgggccacatgtgacagaggtggagggcacatccttcctgacaggcatcatctcctggggagaggagtgtgccatgaagggcaagtatggcatctacaccaaggtgtccaggtatgtcaactggatcaaggagaagaccaagctgacctga

SEQ ID NO: 35 - DNA, 1386 bp, Artificial Sequence
  Synthetic FIX Optimized Sequence Beta 4-No Padua
atgcagagagtgaacatgatcatggctgagtcccctggcctgatcaccatctgcctgctgggctacctgctgtctgctgagtgcacagtgttcctggatcatgagaatgccaacaagatcctgaacagacccaagagatacaactctggcaagctggaggagtttgtgcagggcaacctggagagagagtgcatggaggagaagtgctcctttgaggaggccagagaggtgtttgagaacacagagagaaccacagagttctggaagcagtatgtggatggagatcagtgtgagtccaacccttgcctgaatggaggctcctgcaaggatgatatcaactcctatgagtgctggtgcccatttggctttgagggcaagaattgtgagctggatgccacctgcaacatcaagaatggcagatgtgagcagttctgcaagaactctgctgataacaaggtggtgtgctcctgcacagagggctacagactggctgagaaccagaagtcctgtgagcctgctgtgcctttcccttgtggcagagtgtctgtgtcccagacctccaagctgaccagagctgagacagtgttccctgatgtggattatgtgaactccacagaggctgagaccatcctggataacatcacccagtccacccagtccttcaatgatttcaccagagtggtgggaggagaggatgccaagcctggccagttcccctggcaggtggtgctgaatggcaaggtggatgccttctgtggaggctccattgtgaatgagaagtggattgtgacagctgcccactgtgtggagacaggagtgaagatcacagtggtggctggagagcacaacattgaggagacagagcacacagagcagaagagaaatgtgatcagaatcatccctcaccacaactacaatgctgccatcaacaagtacaaccatgatattgccctgctggagctggatgagcccctggtgctgaactcctatgtgacccctatctgcattgccaacaaggagtacaccaacatcttcctgaagtttggctctggctatgtgtctggctggggcagagtgttccacagaggcagatctgcctctgtgctgcagtacctgagagtgcctctggtggatagagccacctgcctgagatccaccaagttcaccatctacaacaacatgttctgtgctggcttccatgagggaggcagagattcctgccagggagattctggaggccctcatgtgacagaggtggagggcacctccttcctgacaggcatcatctcctggggagaggagtgtgccatgaagggcaagtatggcatctacaccaaggtgtccagatatgtgaactggatcaaggagaagaccaagctgacctga

SEQ ID NO: 36 - DNA, 1386 bp, Artificial Sequence
  Synthetic FIX Optimized Sequence Delta 1-No Padua
atgcagagggtgaacatgatcatggcagagagccctggcctgatcaccatctgcctgctgggctacctgctgtcagcagagtgcacagtgttcctggaccatgagaatgccaacaagatcctgaacaggcccaagaggtacaacagtggcaagctggaggagtttgtccagggcaacctggagagggagtgcatggaggagaagtgctcctttgaggaggccagggaggtgtttgagaacacagagaggacaacagagttctggaagcagtatgttgatggtgaccagtgtgagagcaacccctgcctgaatggtggctcctgcaaggatgacatcaacagctatgagtgctggtgcccctttggctttgagggcaagaactgtgagctggatgccacctgcaacatcaagaatggcaggtgtgagcagttctgcaagaacagtgctgacaacaaggtggtgtgctcctgcacagagggctacaggctggcagagaaccagaagagctgtgagcctgctgtccccttcccctgtggcagggtgtcagtgtcccagaccagcaagctgaccagggcagagacagtgttccctgatgttgactatgtcaacagcacagaggcagagaccatcctggacaacatcacccagagcacccagtccttcaatgacttcaccagggtggtggggggggaggatgccaagcctggccagttcccctggcaggtggtgctgaatggcaaggtggatgccttctgtggtggcagcattgtcaatgagaagtggatagtgacagctgcccactgtgtggagacaggggtgaagataacagtggtggcaggggagcacaacattgaggagacagagcacacagagcagaagaggaatgtcatcaggatcatcccccaccacaactacaatgctgccatcaacaagtacaaccatgacattgccctgctggagctggacaagcccctgaccctgaacagctatgtcacccccatctgcattgccaacagggagtacaccaacatcttcctgaagtttggcagtggctatgtctctggctggggcagggtgttccacaggggcaggagtgccagtgtcctgcagtacctgagggtgcccctggtggacagggccacctgcctgaggagcaccaagttcaccatctacaacaacatgttctgtgctggcttccatgaggggggcagggacagctgccagggggacagtggtggcccccatgtgacagaggtggagggcacctccttcctgacaggcatcatctcctggggggaggagtgtgccatgaagggcaagtatggcatctacaccaaggtgagcaggtatgtcaactggatcaaggagaagaccaagctgacatga

SEQ ID NO: 37 - DNA, 1386 bp, Artificial Sequence
  Synthetic FIX Optimized Sequence Delta 2-No Padua
atgcagagggtcaacatgatcatggctgagtcacctggcctgatcaccatctgcctgctgggctacctgctgtctgctgagtgcacagtgttcctggaccatgagaatgccaacaagatcctgaacaggcccaagaggtacaacagtggcaagctggaggagtttgtgcagggcaacctggagagggagtgcatggaggagaagtgcagctttgaggaggccagggaggtctttgagaacacagagaggaccacagagttctggaagcagtatgtggatggtgaccagtgtgagagcaacccctgcctgaatggtggctcctgcaaggatgacatcaacagctatgagtgctggtgcccctttggctttgagggcaagaactgtgagctggatgccacctgcaacatcaagaatggcaggtgtgagcagttctgcaagaacagtgctgacaacaaggtggtgtgcagctgcacagagggctacaggctggctgagaaccagaagtcctgtgagcctgctgtgccattcccctgtggcagggtcagtgtcagccagaccagcaagctgaccagggctgagacagtgttccctgatgtggactatgtgaacagcacagaggctgagaccatcctggacaacatcacccagagcacccagagcttcaatgacttcaccagagtggtgggtggtgaggatgccaagcctggccagttcccctggcaggtggtgctcaatggcaaggtggatgccttctgtggtggcagcattgtcaatgagaagtggattgtcacagctgcacactgtgtggagacaggtgtcaagatcacagtggtggctggtgagcacaacattgaggagacagagcacacagagcagaagaggaatgtgatcaggatcatcccacaccacaactacaatgctgccatcaacaagtacaaccatgacattgcactgctggagctggacaagccactgaccctgaacagctatgtgacccccatctgcattgccaacagggagtacaccaacatcttcctgaagtttggctctggctatgtcagtggctggggcagggtgttccacagaggcaggtctgccagtgtgctgcagtacctgagggtgccactggtggacagggccacctgcctcaggtccaccaagttcaccatctacaacaacatgttctgtgctggcttccatgagggtggcagggacagctgccagggtgactctggtggcccacatgtgacagaggtggagggcaccagcttcctgacaggcatcatcagctggggtgaggagtgtgccatgaagggcaagtatggcatctacaccaaggtcagcaggtatgtcaactggatcaaggagaagaccaagctgacctga

SEQ ID NO: 38 - DNA, 1386 bp, Artificial Sequence
  Synthetic FIX Optimized Sequence Delta 3-No Padua
atgcagagggtcaacatgatcatggctgagtcaccaggcttgatcaccatctgcctgctgggctacctgctgtcagctgagtgcacagtgtttcttgaccatgagaatgccaacaagatcctcaacaggcccaagaggtacaacagtggcaagctggaggagtttgtccaaggcaacctggagagagagtgcatggaggagaagtgcagctttgaggaggccagagaggtgtttgagaacactgagaggaccactgagttctggaagcagtatgtggatggtgaccagtgtgagtccaacccctgtctcaatgggggcagctgcaaggatgacatcaacagctatgagtgctggtgcccctttggctttgagggcaagaactgtgagcttgatgccacctgcaacatcaagaatggcaggtgtgagcagttctgcaagaactcagctgacaacaaggtggtgtgcagctgcactgagggctacaggctggctgagaaccagaagagctgtgagccagctgtgcccttcccctgtggcagggtgagtgtgagccagaccagcaagctgaccagggctgagacagtgttccctgatgttgactatgtcaacagcactgaggctgagaccatcttggacaacatcactcagtcaacccagagcttcaatgacttcaccagggtggtggggggtgaggatgctaagccaggccagttcccctggcaggtggtgctcaatggcaaggttgatgccttctgtggtggctcaattgtcaatgagaagtggattgtgacagctgcccactgtgttgagactggggtcaagatcacagtggtggctggtgagcacaacattgaggagactgagcacactgagcagaagaggaatgtgatcaggatcatccctcatcacaactacaatgctgccatcaacaagtacaatcatgacatagccctgcttgagcttgacaagcccctgaccctcaacagctatgtcacccccatctgcattgccaacagggagtacaccaacatcttcctcaagtttggctcaggctatgtctcaggctggggcagggtgttccacaggggcaggtcagcctcagtgctgcagtacctcagggtgccactggtggacagggccacctgcctgaggtcaaccaagttcaccatctacaacaacatgttctgtgctggcttccatgaggggggcagagacagctgccagggtgactcaggtgggccccatgtgactgaggttgagggcaccagcttcctcactggcatcatcagctggggtgaggagtgtgccatgaagggcaagtatggcatctacaccaaggtgagcaggtatgtcaactggatcaaggagaagaccaagctgacctga

SEQ ID NO: 39 - DNA, 1386 bp, Artificial Sequence
  Synthetic FIX Optimized Sequence Delta 4-No Padua
atgcagagagtcaacatgatcatggctgagtcccctgggctgatcaccatctgcctgctgggctacctgctgtctgctgagtgcacagtcttcctggaccatgagaatgccaacaagatcctcaacaggcccaagaggtacaactcaggcaagctggaggagtttgtccagggcaacctggagagagagtgcatggaggagaagtgctcctttgaggaggccagagaggtctttgagaacacagagagaaccacagagttctggaagcagtatgtggatggtgaccagtgtgagagcaacccctgcctgaatggaggctcctgcaaggatgacatcaacagctatgagtgctggtgcccctttggctttgagggcaagaactgtgagctggatgccacctgcaacatcaagaatggcagatgtgagcagttctgcaagaactctgcagacaacaaggtggtctgcagctgcacagagggctacaggctggctgagaaccagaagagctgtgagcctgctgtgcccttcccctgtggcagagtgtcagtcagccagaccagcaagctgaccagagcagagacagtcttccctgatgtggactatgtgaactccacagaggcagagaccatcctggataacatcacccagagcactcagagcttcaatgatttcaccagggtggtgggtggtgaggatgccaagcctggccagttcccctggcaggtggtcctgaatggcaaggtggatgccttctgtggaggctccattgtgaatgagaagtggattgtcactgctgctcactgtgtggagacaggagtgaagatcactgtggtggctggagagcacaacatagaggagacagagcacacagagcagaagagaaatgtcatcaggatcatcccccatcacaactacaatgcagccatcaacaagtacaaccatgacattgccctgctggagctggacaagcccctcaccctgaactcctatgtgactcccatctgcattgccaacagagagtacaccaacatcttcctgaagtttggctctggctatgtgtctggctggggcagagtgttccacaggggcaggtctgccagtgtgctgcagtacctcagagtgcccctggtggacagagccacctgcctgaggagcaccaagttcaccatctacaacaacatgttctgtgctggcttccatgagggaggcagagatagctgccagggtgactctggaggcccccatgtgactgaggtggagggcacctccttcctgacaggcatcatctcctggggggaggagtgtgccatgaagggcaagtatggcatctacaccaaggtgagcaggtatgtcaactggatcaaggagaagaccaagctgacctga

SEQ ID NO: 40 - DNA, 1386 bp, Artificial Sequence
  Synthetic FIX Optimized Sequence Gamma 1-No Padua
atgcagagggtcaacatgatcatggcagagtcccctgggctcatcaccatctgcctcctggggtacctgctcagtgctgagtgcacagtgttcctggaccatgagaatgccaacaagatcctgaacaggcccaagaggtacaactctgggaaactggaggagtttgtccaggggaacctggagagggagtgcatggaggagaagtgctcctttgaggaggccagggaggtctttgagaacacagagaggaccactgagttctggaagcagtatgtggatggggaccagtgtgagtccaacccctgcctcaatgggggcagctgcaaggatgacatcaactcctatgagtgctggtgcccctttggctttgagggcaagaactgtgagctggatgccacctgcaacatcaagaatggcagatgtgagcagttctgcaagaactctgctgacaacaaggtggtgtgcagctgcactgagggctacaggctggctgagaaccagaagtcctgtgagccagctgtgcccttcccctgtggcagagtctcagtgtcccagacctccaagctgaccagagctgagacagtgttcccagatgtggactatgtgaactccacagaggctgagaccatcctggacaacatcacccagagcacccagagcttcaatgacttcaccagggtggtgggaggagaagatgccaagcctgggcagttcccctggcaggtggtgctcaatggcaaagtggatgccttctgtggaggctccattgtcaatgagaagtggattgtcacagctgcccactgtgtggagacaggggtcaagatcacagtggtggcaggggagcacaacattgaggagacagagcacacagagcagaagaggaatgtgatcaggatcatcccccaccacaactacaatgctgccatcaacaagtacaaccatgacattgccctgctggagctggacaagcccctggtgctgaactcctatgtgacccccatctgcattgccaacaaggagtacaccaacatcttcctgaagtttggctctgggtatgtctcaggctggggcagagtgttccacaggggcagaagtgcctcagtgctgcagtacctcagggtgcccctggtggacagggccacttgcctcaggagcaccaagttcaccatctacaacaacatgttctgtgcagggttccatgaggggggcagggactcctgccagggggactctgggggcccccatgtcacagaggtggagggcaccagtttcctcacagggatcatctcctggggtgaggagtgtgccatgaagggcaagtatggcatctacaccaaggtgtccaggtatgtgaactggatcaaggagaagaccaagctgacctga

SEQ ID NO: 41 - DNA, 1386 bp, Artificial Sequence
  Synthetic FIX Optimized Sequence Gamma 2-No Padua
atgcagagggtcaacatgatcatggctgagagccctggcctcatcaccatctgcctgctgggctacctgctgtcagctgagtgcacagtgttcctggaccatgagaatgccaacaagatcctcaacaggcccaagaggtacaacagtggcaagctggaggagtttgtgcagggcaacctggagagggagtgcatggaggagaagtgcagctttgaggaggccagggaggtgtttgagaacacagagaggacaacagagttctggaagcagtatgttgatggtgaccagtgtgagagcaacccctgcctgaatgggggcagctgcaaggatgacatcaacagctatgagtgctggtgcccctttggctttgagggcaagaactgtgagcttgatgccacctgcaacatcaagaatggcaggtgtgagcagttctgcaagaacagtgctgacaacaaggtggtgtgcagctgcacagagggctacaggctggctgagaaccagaagagctgtgagcctgctgtgcccttcccctgtggcagggtgtctgtgagccagacaagcaagctgaccagggctgagacagtgttccctgatgttgactatgtcaacagcacagaggctgagaccatcctggacaacatcacacagtcaacacagagcttcaatgacttcaccagggtggtggggggggaggatgccaagcctggccagttcccctggcaggtggtgctgaatggcaaggttgatgccttctgtgggggcagcattgtcaatgagaagtggattgtgacagctgcccactgtgttgagacaggggtcaagatcacagtggtggctggtgagcacaacattgaggagacagagcacacagagcagaagaggaatgtcatcaggatcatcccccaccacaactacaatgctgccatcaacaagtacaaccatgacatagccctgctggagcttgacaagcccctggtgctcaacagctatgtgacccccatctgtatagccaacaaggagtacaccaacatcttcctcaagtttggctcaggctatgtgtcaggctggggcagggtgttccacaggggcaggtcagcctctgtgctgcagtacctgagggtgcccctggttgacagggccacctgcctgaggagcaccaagttcaccatctacaacaacatgttctgtgctggcttccatgaggggggcagggacagctgccagggtgacagtgggggccctcatgtgacagaggttgagggcaccagcttcctgacaggcatcatcagctggggggaggagtgtgccatgaagggcaagtatggcatctacaccaaggtgagcaggtatgtcaactggatcaaggagaagaccaagctgacatga

SEQ ID NO: 42 - DNA, 1386 bp, Artificial Sequence
  Synthetic FIX Optimized Sequence Gamma 3-No Padua
atgcagagagtgaacatgatcatggctgagtcacctggactgatcaccatctgcctgctgggctacctgctgtctgctgagtgcacagtcttcctggatcatgagaatgccaacaagatcctgaacagacctaagaggtacaactctggcaagctggaggagtttgtgcagggcaacctggagagagagtgcatggaggagaagtgcagctttgaggaggccagagaggtgtttgagaacacagagaggaccacagagttctggaagcagtatgtggatggtgatcagtgtgagagcaacccctgcctgaatggaggcagctgcaaggatgatatcaactcctatgagtgctggtgcccctttggctttgagggcaagaactgtgagctggatgccacctgcaacatcaagaatggcagatgtgagcagttctgcaagaacagtgctgataacaaggtggtgtgcagctgcacagagggctacagactggctgagaaccagaagagctgtgagccagctgtgcccttcccctgtggcagagtgtcagtgtcacagaccagcaagctgaccagagctgagacagtcttccctgatgtggactatgtgaacagcacagaggctgagaccatcctggataacatcacccagtccacccagtcattcaatgacttcaccagagtggtgggaggagaggatgccaagcctggccagttcccctggcaggtggtgctgaatggcaaggtggatgcattctgtggaggcagcattgtgaatgagaagtggattgtgacagctgcccactgtgtggagacaggtgtgaagatcacagtggtggctggagagcacaacattgaggagacagagcacacagagcagaagaggaatgtgatcaggatcatccctcaccacaactacaatgctgccatcaacaagtacaaccatgatattgcactgctggagctggataagccactggtgctgaacagctatgtgacacctatctgcattgccaacaaggagtacaccaacatcttcctgaagtttggcagtggctatgtgtcaggctggggcagagtgttccacagaggcagatcagcctctgtgctgcagtacctgagagtgccactggtggatagagccacctgcctgaggagcaccaagttcaccatctacaacaacatgttctgtgctggcttccatgagggaggcagagatagctgccagggtgacagtggtggccctcatgtgacagaggtggagggcacctccttcctgactggcatcatctcctggggagaggagtgtgccatgaagggcaagtatggcatctacaccaaggtgagcagatatgtgaactggatcaaggagaagaccaagctgacatga

SEQ ID NO: 43 - DNA, 1386 bp, Artificial Sequence
  Synthetic FIX Optimized Sequence Gamma 4-No Padua
atgcagagagtgaacatgatcatggctgagagccctggcctgatcaccatctgcctgctgggctacctgctgtctgctgagtgcacagtgttcctggatcatgagaatgccaacaagatcctgaacagacccaagagatacaactctggcaagctggaggagtttgtgcagggcaacctggagagagagtgcatggaggagaagtgctcctttgaggaggccagagaggtgtttgagaacacagagagaaccacagagttctggaagcagtatgtggatggagatcagtgtgagtccaacccttgcctgaatggaggctcctgcaaggatgatatcaactcctatgagtgctggtgcccttttggctttgagggcaagaactgtgagctggatgccacctgcaacatcaagaatggcagatgtgagcagttctgcaagaactctgctgataacaaggtggtgtgctcctgcacagagggctacagactggctgagaaccagaagtcctgtgagcctgctgtgcccttcccttgtggcagagtgtctgtgtcccagacctccaagctgaccagagctgagacagtgttccctgatgtggattatgtgaactccacagaggctgagaccatcctggataacatcacccagtccacccagtccttcaatgatttcaccagagtggtgggaggagaggatgccaagcctggccagttcccttggcaggtggtgctgaatggcaaggtggatgccttctgtggaggctccattgtgaatgagaagtggattgtgacagctgcccactgtgtggagacaggagtgaagatcacagtggtggctggagagcacaacattgaggagacagagcacacagagcagaagagaaatgtgatcagaatcatccctcaccacaactacaatgctgccatcaacaagtacaaccatgatattgccctgctggagctggataagcctctggtgctgaactcctatgtgacacccatctgcattgccaacaaggagtacaccaacatcttcctgaagtttggctctggctatgtgtctggctggggcagagtgttccacagaggcagatctgcctctgtgctgcagtacctgagagtgcctctggtggatagagccacctgcctgagatccaccaagttcaccatctacaacaacatgttctgtgctggcttccatgagggaggcagagattcctgccagggagattctggaggccctcatgtgacagaggtggagggcacctccttcctgacaggcatcatctcctggggagaggagtgtgccatgaagggcaagtatggcatctacaccaaggtgtccagatatgtgaactggatcaaggagaagaccaagctgacctga

SEQ ID NO: 44 - DNA, 1386 bp, Artificial Sequence
  Synthetic FIX Optimized Sequence (With Padua), Gamma 22 Padua
atgcagagggtcaacatgatcatggcagagtcccctgggctcatcaccatctgcctcctggggtacctgctcagtgctgagtgcacagtgttcctggaccatgagaatgccaacaagatcctgaacaggcccaagaggtacaactctgggaaactggaggagtttgtccaggggaacctggagagggagtgcatggaggagaagtgctcctttgaggaggccagggaggtctttgagaacacagagaggaccactgagttctggaagcagtatgtggatggggaccagtgtgagtccaacccctgcctcaatgggggcagctgcaaggatgacatcaactcctatgagtgctggtgcccctttggctttgagggcaagaactgtgagctggatgccacctgcaacatcaagaatggcagatgtgagcagttctgcaagaactctgctgacaacaaggtggtgtgcagctgcactgagggctacaggctggctgagaaccagaagtcctgtgagccagctgtgcccttcccctgtggcagagtctcagtgtcccagacctccaagctgaccagagctgagacagtgttcccagatgtggactatgtgaactccacagaggctgagaccatcctggacaacatcacccagagcacccagagcttcaatgacttcaccagggtggtgggaggagaagatgccaagcctgggcagttcccctggcaggtggtgctcaatggcaaagtggatgccttctgtggaggctccattgtcaatgagaagtggattgtcacagctgcccactgtgtggagacaggggtcaagatcacagtggtggcaggggagcacaacattgaggagacagagcacacagagcagaagaggaatgtgatcaggatcatcccccaccacaactacaatgctgccatcaacaagtacaaccatgacattgccctgctggagctggacaagcccctggtgctgaactcctatgtgacccccatctgcattgccaacaaggagtacaccaacatcttcctgaagtttggctctgggtatgtctcaggctggggcagagtgttccacaggggcagaagtgcctcagtgctgcagtacctcagggtgcccctggtggacagggccacttgcctcctgagcaccaagttcaccatctacaacaacatgttctgtgcagggttccatgaggggggcagggactcctgccagggggactctgggggcccccatgtcacagaggtggagggcaccagtttcctcacagggatcatctcctggggtgaggagtgtgccatgaagggcaagtatggcatctacaccaaggtgtccaggtatgtgaactggatcaaggagaagaccaagctgacctga

SEQ ID NO: 45 - DNA, 1386 bp, Artificial Sequence
  Synthetic FIX Optimized Sequence (With Padua), Gamma 3 Padua
atgcagagggtcaacatgatcatggctgagagccctggcctcatcaccatctgcctgctgggctacctgctgtcagctgagtgcacagtgttcctggaccatgagaatgccaacaagatcctcaacaggcccaagaggtacaacagtggcaagctggaggagtttgtgcagggcaacctggagagggagtgcatggaggagaagtgcagctttgaggaggccagggaggtgtttgagaacacagagaggacaacagagttctggaagcagtatgttgatggtgaccagtgtgagagcaacccctgcctgaatgggggcagctgcaaggatgacatcaacagctatgagtgctggtgcccctttggctttgagggcaagaactgtgagcttgatgccacctgcaacatcaagaatggcaggtgtgagcagttctgcaagaacagtgctgacaacaaggtggtgtgcagctgcacagagggctacaggctggctgagaaccagaagagctgtgagcctgctgtgcccttcccctgtggcagggtgtctgtgagccagacaagcaagctgaccagggctgagacagtgttccctgatgttgactatgtcaacagcacagaggctgagaccatcctggacaacatcacacagtcaacacagagcttcaatgacttcaccagggtggtggggggggaggatgccaagcctggccagttcccctggcaggtggtgctgaatggcaaggttgatgccttctgtgggggcagcattgtcaatgagaagtggattgtgacagctgcccactgtgttgagacaggggtcaagatcacagtggtggctggtgagcacaacattgaggagacagagcacacagagcagaagaggaatgtcatcaggatcatcccccaccacaactacaatgctgccatcaacaagtacaaccatgacatagccctgctggagcttgacaagcccctggtgctcaacagctatgtgacccccatctgtatagccaacaaggagtacaccaacatcttcctcaagtttggctcaggctatgtgtcaggctggggcagggtgttccacaggggcaggtcagcctctgtgctgcagtacctgagggtgcccctggttgacagggccacctgcctgctgagcaccaagttcaccatctacaacaacatgttctgtgctggcttccatgaggggggcagggacagctgccagggtgacagtgggggccctcatgtgacagaggttgagggcaccagcttcctgacaggcatcatcagctggggggaggagtgtgccatgaagggcaagtatggcatctacaccaaggtgagcaggtatgtcaactggatcaaggagaagaccaagctgacatga

SEQ ID NO: 46 - DNA, 1386 bp, Artificial Sequence
  Synthetic FIX Optimized Sequence (With Padua), Gamma 87 Padua
atgcagagagtgaacatgatcatggctgagtcacctggactgatcaccatctgcctgctgggctacctgctgtctgctgagtgcacagtcttcctggatcatgagaatgccaacaagatcctgaacagacctaagaggtacaactctggcaagctggaggagtttgtgcagggcaacctggagagagagtgcatggaggagaagtgcagctttgaggaggccagagaggtgtttgagaacacagagaggaccacagagttctggaagcagtatgtggatggtgatcagtgtgagagcaacccctgcctgaatggaggcagctgcaaggatgatatcaactcctatgagtgctggtgcccctttggctttgagggcaagaactgtgagctggatgccacctgcaacatcaagaatggcagatgtgagcagttctgcaagaacagtgctgataacaaggtggtgtgcagctgcacagagggctacagactggctgagaaccagaagagctgtgagccagctgtgcccttcccctgtggcagagtgtcagtgtcacagaccagcaagctgaccagagctgagacagtcttccctgatgtggactatgtgaacagcacagaggctgagaccatcctggataacatcacccagtccacccagtcattcaatgacttcaccagagtggtgggaggagaggatgccaagcctggccagttcccctggcaggtggtgctgaatggcaaggtggatgcattctgtggaggcagcattgtgaatgagaagtggattgtgacagctgcccactgtgtggagacaggtgtgaagatcacagtggtggctggagagcacaacattgaggagacagagcacacagagcagaagaggaatgtgatcaggatcatccctcaccacaactacaatgctgccatcaacaagtacaaccatgatattgcactgctggagctggataagccactggtgctgaacagctatgtgacacctatctgcattgccaacaaggagtacaccaacatcttcctgaagtttggcagtggctatgtgtcaggctggggcagagtgttccacagaggcagatcagcctctgtgctgcagtacctgagagtgccactggtggatagagccacctgcctgctgagcaccaagttcaccatctacaacaacatgttctgtgctggcttccatgagggaggcagagatagctgccagggtgacagtggtggccctcatgtgacagaggtggagggcacctccttcctgactggcatcatctcctggggagaggagtgtgccatgaagggcaagtatggcatctacaccaaggtgagcagatatgtgaactggatcaaggagaagaccaagctgacatga

SEQ ID NO: 47 - DNA, 1386 bp, Artificial Sequence
  Synthetic FIX Optimized Sequence (With Padua), Gamma 2 Padua
atgcagagagtgaacatgatcatggctgagagccctggcctgatcaccatctgcctgctgggctacctgctgtctgctgagtgcacagtgttcctggatcatgagaatgccaacaagatcctgaacagacccaagagatacaactctggcaagctggaggagtttgtgcagggcaacctggagagagagtgcatggaggagaagtgctcctttgaggaggccagagaggtgtttgagaacacagagagaaccacagagttctggaagcagtatgtggatggagatcagtgtgagtccaacccttgcctgaatggaggctcctgcaaggatgatatcaactcctatgagtgctggtgcccttttggctttgagggcaagaactgtgagctggatgccacctgcaacatcaagaatggcagatgtgagcagttctgcaagaactctgctgataacaaggtggtgtgctcctgcacagagggctacagactggctgagaaccagaagtcctgtgagcctgctgtgcccttcccttgtggcagagtgtctgtgtcccagacctccaagctgaccagagctgagacagtgttccctgatgtggattatgtgaactccacagaggctgagaccatcctggataacatcacccagtccacccagtccttcaatgatttcaccagagtggtgggaggagaggatgccaagcctggccagttcccttggcaggtggtgctgaatggcaaggtggatgccttctgtggaggctccattgtgaatgagaagtggattgtgacagctgcccactgtgtggagacaggagtgaagatcacagtggtggctggagagcacaacattgaggagacagagcacacagagcagaagagaaatgtgatcagaatcatccctcaccacaactacaatgctgccatcaacaagtacaaccatgatattgccctgctggagctggataagcctctggtgctgaactcctatgtgacacccatctgcattgccaacaaggagtacaccaacatcttcctgaagtttggctctggctatgtgtctggctggggcagagtgttccacagaggcagatctgcctctgtgctgcagtacctgagagtgcctctggtggatagagccacctgcctgctgtccaccaagttcaccatctacaacaacatgttctgtgctggcttccatgagggaggcagagattcctgccagggagattctggaggccctcatgtgacagaggtggagggcacctccttcctgacaggcatcatctcctggggagaggagtgtgccatgaagggcaagtatggcatctacaccaaggtgtccagatatgtgaactggatcaaggagaagaccaagctgacctga

SEQ ID NO: 48 - DNA, 1386 bp, Artificial Sequence
  Synthetic FIX Optimized Sequence (With Padua), Beta 82 Padua
atgcagagggtgaacatgatcatggctgagagccctgggctgatcaccatctgcctgctggggtacctgctgagtgctgagtgcactgtgttcctggaccatgagaatgccaacaagatcctcaacaggcccaagaggtacaacagtggcaagctggaggagtttgtgcagggcaacctggagagggagtgcatggaggagaagtgcagctttgaggaggccagggaggtgtttgagaacacagagaggaccacagagttctggaagcagtatgtggatggggaccagtgtgagtccaacccctgcctgaatgggggcagctgcaaggatgacatcaacagctatgagtgctggtgcccctttggctttgagggcaagaactgtgagctggatgccacctgcaacatcaagaatggcaggtgtgagcagttctgcaagaacagtgctgacaacaaggtggtgtgcagctgcactgagggctacaggctggctgagaaccagaagtcctgtgagcctgcagtgcccttcccctgtggcagggtgtctgtgtcccagaccagcaagctcaccagggctgagactgtgttccctgatgtggactatgtcaacagcacagaggctgagaccatcctggacaacatcacccagagcacccagagcttcaatgacttcaccagggtggtggggggggaggatgccaagccagggcagttcccctggcaggtggtgctgaatggcaaggtggatgccttctgtgggggcagcattgtcaatgagaagtggattgtcacagcagcccactgtgtggagactggggtgaagatcacagtggtggctggggagcacaacattgaggagacagagcacacagagcagaagaggaatgtcatcaggatcatcccccaccacaactacaatgctgccatcaacaagtacaaccatgacattgccctgctggagctggatgagcccctggtgctgaacagctatgtcacccccatctgcattgccaacaaggagtacaccaacatcttcctgaagtttggcagtggctatgtcagtggctggggcagggtgttccacaggggcaggtctgccagtgtgctgcagtacctgagggtgcccctggtggacagggccacctgcctgctgtccaccaagttcaccatctacaacaacatgttctgtgctggcttccatgaggggggcagggacagctgccagggggactctgggggcccccatgtcactgaggtggagggcaccagcttcctgacaggcatcatcagctggggggaggagtgtgccatgaagggcaagtatggcatctacaccaaggtcagcaggtatgtgaactggatcaaggagaagaccaagctcacctga

SEQ ID NO: 49 - DNA, 1386 bp, Artificial Sequence
  Synthetic FIX Optimized Sequence (With Padua), Beta 81 Padua
atgcagagggtgaacatgatcatggctgagtcccctggcctgatcaccatctgcctgctgggctacctgctgtctgctgagtgcacagtgttcctggaccatgagaatgccaacaagatcctgaacaggcccaagaggtacaactctggcaagctggaggagtttgtgcagggcaacctggagagggagtgcatggaggagaagtgctcctttgaggaggccagggaggtgtttgagaacacagagaggaccacagagttctggaagcagtatgtggatggagaccagtgtgagtccaacccttgcctgaatggaggctcctgcaaggatgacatcaactcctatgagtgctggtgcccctttggctttgagggcaagaactgtgagctggatgccacctgcaacatcaagaatggcaggtgtgagcagttctgcaagaactctgctgacaacaaggtggtgtgctcctgcacagagggctacaggctggctgagaaccagaagtcctgtgagccagctgtgcccttcccttgtggcagggtgtctgtgtcccagacctccaagctgaccagggctgagacagtgttccctgatgtggactatgtgaactccacagaggctgagaccatcctggacaacatcacccagtccacccagtccttcaatgacttcaccagggtggtgggaggtgaggatgccaagcctggccagttcccttggcaggtggtgctgaatggcaaggtggatgccttctgtggaggctccattgtgaatgagaagtggattgtgacagctgcccactgtgtggagacaggagtgaagatcacagtggtggctggtgagcacaacattgaggagacagagcacacagagcagaagaggaatgtgatcaggatcatcccccaccacaactacaatgctgccatcaacaagtacaaccatgacattgccctgctggagctggatgagcccctggtgctgaactcctatgtgacccccatctgcattgccaacaaggagtacaccaacatcttcctgaagtttggctctggctatgtgtcaggatggggcagggtgttccacaggggcaggtctgcctctgtgctgcagtacctgagggtgcccctggtggacagggccacctgcctgctgtccaccaagttcaccatctacaacaacatgttctgtgctggcttccatgagggaggcagggactcctgccagggtgactcaggaggcccccatgtgacagaggtggagggcacctccttcctgacaggcatcatcagctggggagaggagtgtgccatgaagggcaagtatggcatctacaccaaggtgtccaggtatgtgaactggatcaaggagaagaccaagctgacctga

SEQ ID NO: 50 - DNA, 1386 bp, Artificial Sequence
  Synthetic FIX Optimized Sequence (With Padua), Beta 23 Padua
atgcagagagtcaacatgatcatggcagagtcacctggactgatcaccatctgcctgctgggctacctgctcagtgcagagtgcacagtcttcctggaccatgagaatgccaacaagatcctgaacagacccaagaggtacaactctggcaagctggaggagtttgtgcagggcaacctggagagagagtgcatggaggagaagtgctcctttgaggaggccagagaggtctttgagaacactgagaggaccactgagttctggaagcagtatgtggatggtgatcagtgtgagtccaacccatgcctgaatggaggctcctgcaaggatgacatcaactcctatgagtgctggtgcccatttggctttgagggcaagaactgtgagctggatgcaacatgcaacatcaagaatggcagatgtgagcagttctgcaagaacagtgcagacaacaaggtggtgtgctcctgcacagagggctacagactggctgagaaccagaagtcatgtgagcctgctgtgcccttcccctgtggcagagtgagtgtgtcccagaccagcaagctgaccagagctgagacagtcttccctgatgtggactatgtgaactccacagaggcagagaccatcctggacaacatcacacagtccacacagtccttcaatgacttcaccagagtggttggtggtgaggatgccaagcctggacagttcccctggcaggtggtgctcaatggcaaggtggatgccttctgtggaggctccattgtcaatgagaagtggattgtcacagctgcccactgtgtggagactggtgtgaagatcactgtggtggctggtgagcacaacattgaggagacagagcacactgagcagaagaggaatgtcatcaggatcatcccacaccacaactacaatgcagccatcaacaagtacaaccatgacattgccctgctggagctggatgagcccctggtgctgaacagctatgtgacccccatctgcatagccaacaaggagtacaccaacatcttcctgaagtttggctctggctatgtgtctggctggggcagagtcttccacagaggcagatctgcctctgtgctgcagtacctgagagtgcctctggtggacagagcaacctgcctgctgagcaccaagttcaccatctacaacaacatgttctgtgcaggcttccatgagggaggcagagactcctgccagggagactctggtgggccacatgtgacagaggtggagggcacatccttcctgacaggcatcatctcctggggagaggagtgtgccatgaagggcaagtatggcatctacaccaaggtgtccaggtatgtcaactggatcaaggagaagaccaagctgacctga

SEQ ID NO: 51 - DNA, 1386 bp, Artificial Sequence
  Synthetic FIX Optimized Sequence (With Padua), Beta 39 Padua
atgcagagagtgaacatgatcatggctgagtcccctggcctgatcaccatctgcctgctgggctacctgctgtctgctgagtgcacagtgttcctggatcatgagaatgccaacaagatcctgaacagacccaagagatacaactctggcaagctggaggagtttgtgcagggcaacctggagagagagtgcatggaggagaagtgctcctttgaggaggccagagaggtgtttgagaacacagagagaaccacagagttctggaagcagtatgtggatggagatcagtgtgagtccaacccttgcctgaatggaggctcctgcaaggatgatatcaactcctatgagtgctggtgcccatttggctttgagggcaagaattgtgagctggatgccacctgcaacatcaagaatggcagatgtgagcagttctgcaagaactctgctgataacaaggtggtgtgctcctgcacagagggctacagactggctgagaaccagaagtcctgtgagcctgctgtgcctttcccttgtggcagagtgtctgtgtcccagacctccaagctgaccagagctgagacagtgttccctgatgtggattatgtgaactccacagaggctgagaccatcctggataacatcacccagtccacccagtccttcaatgatttcaccagagtggtgggaggagaggatgccaagcctggccagttcccctggcaggtggtgctgaatggcaaggtggatgccttctgtggaggctccattgtgaatgagaagtggattgtgacagctgcccactgtgtggagacaggagtgaagatcacagtggtggctggagagcacaacattgaggagacagagcacacagagcagaagagaaatgtgatcagaatcatccctcaccacaactacaatgctgccatcaacaagtacaaccatgatattgccctgctggagctggatgagcccctggtgctgaactcctatgtgacccctatctgcattgccaacaaggagtacaccaacatcttcctgaagtttggctctggctatgtgtctggctggggcagagtgttccacagaggcagatctgcctctgtgctgcagtacctgagagtgcctctggtggatagagccacctgcctgctgtccaccaagttcaccatctacaacaacatgttctgtgctggcttccatgagggaggcagagattcctgccagggagattctggaggccctcatgtgacagaggtggagggcacctccttcctgacaggcatcatctcctggggagaggagtgtgccatgaagggcaagtatggcatctacaccaaggtgtccagatatgtgaactggatcaaggagaagaccaagctgacctga

SEQ ID NO: 52 - protein, 461 aa, Artificial Sequence
  Synthetic Alpha (no Padua)
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDATCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIANKEYTNIFLKFGSGYVSGWGRVFHKGRSASVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 53 - protein, 461 aa, Artificial Sequence
  Synthetic Beta (no Padua)
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDATCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIANKEYTNIFLKFGSGYVSGWGRVFHRGRSASVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 54 - protein, 461 aa, Artificial Sequence
  Synthetic Delta (no Padua)
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDATCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDKPLTLNSYVTPICIANREYTNIFLKFGSGYVSGWGRVFHRGRSASVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 55 - protein, 461 aa, Artificial Sequence
  Synthetic Gamma (no Padua)
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDATCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDKPLVLNSYVTPICIANKEYTNIFLKFGSGYVSGWGRVFHRGRSASVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 56 - protein, 461 aa, Artificial Sequence
  Synthetic Gamma (with Padua)
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDATCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDKPLVLNSYVTPICIANKEYTNIFLKFGSGYVSGWGRVFHRGRSASVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT

SEQ ID NO: 57 - protein, 461 aa, Artificial Sequence
  Synthetic Beta (with Padua)
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDATCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIANKEYTNIFLKFGSGYVSGWGRVFHRGRSASVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLT